(19) United States
(12) Patent Application Publication
(10) Pub. No.: US 2009/0123419 A1
(43) Pub. Date: May 14, 2009

(54) TREATMENT OF UTERINE CANCER AND OVARIAN CANCER WITH A PARP INHIBITOR ALONE OR IN COMBINATION WITH ANTI-TUMOR AGENTS

(75) Inventors: Barry M. Sherman, Hillsborough, CA (US); Charles Bradley, Half Moon Bay, CA (US); Valeria Ossovskaya, San Francisco, CA (US)

Correspondence Address: WILSON SONSINI GOODRICH & ROSATI, 650 PAGE MILL ROAD, PALO ALTO, CA, 94304-1050, US

(73) Assignee: BiPar Sciences

(21) Appl. No.: 12/269,833

(22) Filed: Nov. 12, 2008

Related U.S. Application Data
(60) Provisional application No. 60/987,335, filed on Nov. 12, 2007.
(60) Provisional application No. 61/012,364, filed on Dec. 7, 2007.
(60) Provisional application No. 61/058,528, filed on Jun. 3, 2008.

(51) Int. Cl. A61K 38/21; A61K 31/166; A61P 35/00; A61K 31/505; A61K 31/337; A61K 31/513; A61K 39/395; C12Q 1/68

(52) U.S. Cl. 424/85.4; 514/619; 514/269; 514/274; 424/138.1; 435/6; 514/449

(57) ABSTRACT

In one aspect, the present invention provides a method of treating uterine cancer, endometrial cancer, or ovarian cancer, comprising administering to a subject at least one PARP inhibitor. In another aspect, the present invention provides a method of treating uterine cancer, endometrial cancer, or ovarian cancer, comprising administering to a subject at least one PARP inhibitor in combination with at least one anti-tumor agent.

### CROSS REFERENCE

This application claims the benefit of U.S. Provisional Application No. 60/987,335, entitled “Treatment of Uterine Cancer with a Combination of a Taxane, a Platinum Complex, and a PARP-1 Inhibitor” filed Nov. 12, 2007 (Attorney Docket No. 28825-742.102); U.S. Provisional Application No. 61/012,364, entitled “Treatment of Cancer with Combinations of Topoisomerase Inhibitors and PARP Inhibitors” filed Dec. 7, 2007 (Attorney Docket No. 28825-747.101); and U.S. Provisional Application No. 61/058,528, entitled “Treatment of Breast, Ovarian, and Uterine Cancer with a PARP Inhibitor” filed Jun. 3, 2008 (Attorney Docket No. 28825-757.101), each of which applications is incorporated herein in its entirety by reference.

### BACKGROUND

Cancer is a group of diseases characterized by aberrant control of cell growth. The annual incidence of cancer is estimated to be in excess of 1.3 million in the United States alone. While surgery, radiation, chemotherapy, and hormones are used to treat cancer, it remains the second leading cause of death in the U.S. It is estimated that over 560,000 Americans will die from cancer each year.

Cancer cells simultaneously activate several pathways that positively and negatively regulate cell growth and cell death. This trait suggests that the modulation of cell death and survival signals could provide new strategies for improving the efficacy of current chemotherapeutic treatments.

Malignant uterine neoplasms containing both carcinomatous and sarcomatous elements are designated in the World Health Organization (WHO) classification of uterine neoplasms as carcinosarcomas. An alternative designation is malignant mixed Mullerian tumor (MMMT). Carcinosarcomas also arise in the ovary/fallopian tube, cervix, peritoneum, and non-gynecologic sites, but with a much lower frequency than in the uterus. These tumors are highly aggressive and have a poor prognosis. Most uterine carcinosarcomas are monoclonal, with the carcinomatous element being the key element and the sarcomatous component derived from the carcinoma or from a stem cell that undergoes divergent differentiation (ie, metaplastic carcinomas). The sarcomatous component is either homologous (composed of tissues normally found in the uterus) or heterologous (containing tissues not normally found in the uterus, most commonly malignant cartilage or skeletal muscle).

Previous studies investigating a number of single agents in carcinosarcoma of the uterus have reported the following response rates: etoposide (6.5%); doxorubicin (9.8%); cisplatin (18%); ifosfamide (32.2%); paclitaxel (18.2%); and topotecan (10%). Thus the three most active agents discovered to date include cisplatin, ifosfamide, and paclitaxel. A randomized phase III trial comparing ifosfamide to ifosfamide plus cisplatin showed an increased response rate (36% vs. 54%), a slight improvement in median progression-free survival (4 vs. 6 months, p=0.02), but no improvement in median survival (7.6 vs. 9.4 months, p=0.07). A second randomized trial evaluated the role of paclitaxel. In this study, patients are randomized to receive ifosfamide versus the combination of ifosfamide plus paclitaxel and showed an increased response rate (29% vs. 45%), improvement in median progression-free survival (3.6 vs. 5.8 months, p=0.03), and improvement in median survival (8.4 vs. 13.5 months, p=0.03). The use of ifosfamide is cumbersome and results in significant toxicity.

In a highly related disease, endometrial carcinoma, there have been several randomized studies addressing the issue of optimal therapy. These studies have focused on three active agents identified in phase II trials: doxorubicin, platinum agents, and paclitaxel. In one study, 281 women are randomized to doxorubicin alone (60 mg/m²) versus doxorubicin (60 mg/m²) plus cisplatin (50 mg/m²) (AP). There is a statistically significant advantage to combination therapy with regard to response rate (RR) (25% versus 42%; p=0.004) and PFS (3.8 vs 5.7 months; HR 0.74 [95% CI 0.58, 0.94; p=0.14), although no difference in OS is observed (9 vs 9.2 months). Paclitaxel had significant single agent activity with a response rate of 36% in advanced or recurrent endometrial cancer. Thus 317 patients are randomized to paclitaxel and doxorubicin or the standard arm. This trial failed to demonstrate a significant difference in RR, PFS, or OS between the two arms, and AP remained the standard of care. However, since both platinum and paclitaxel had demonstrated high single agent activity, there is as strong interest in including paclitaxel and cisplatin in a front-line regimen for advanced and recurrent endometrial cancer. Subsequently, another study randomized 263 patients to AP versus TAP: doxorubicin (45 mg/m²) and cisplatin (50 mg/m²) on day 1, followed by paclitaxel (160 mg/m² IV over 3 hours) on day 2 (with G-CSF support). TAP is superior to AP in terms of ORR (57% vs 34%; p<0.01), median PFS (8.3 vs 5.3 months; p<0.01) and OS with a median of 15.3 (TAP) versus 12.3 months (AP) (p=0.037). This improved efficacy came at the cost of increased toxicity.

Although there are limited therapeutic options for cancer treatment, variants of cancers, including recurrent, advanced or persistent uterine cancer and BRCA-deficient ovarian cancer, are especially difficult because they can be refractory to standard chemotherapeutic or hormonal treatment. There is thus a need for an effective treatment for cancer in general, and cancer variants in particular. The present invention addresses these needs and provides related advantages as well.

### SUMMARY OF THE INVENTION

In one aspect, the present invention provides a method of treating uterine cancer or ovarian cancer in a patient, comprising administering to the patient at least one PARP inhibitor. In another aspect, the present invention provides a method of treating ovarian cancer or uterine cancer in a patient in need of such treatment, comprising: (a) obtaining a sample from the patient; (b) testing the sample to determine whether the patient is BRCA deficient; (c) if the testing indicates that the patient is BRCA-deficient, treating the patient with at least one PARP inhibitor. In another aspect, the present invention provides a method of treating ovarian cancer or uterine cancer in a patient in need of such treatment, comprising: (a) obtaining a sample from the patient; (b) testing the sample to determine a level of PARP expression in the sample; (c) determining whether the PARP expression exceeds a predetermined level, and if so, administering to the patient at least one PARP inhibitor.

In practicing any of the methods disclosed herein, in some embodiments, at least one therapeutic effect is obtained, said at least one therapeutic effect being reduction in size of a uterine tumor or an ovarian tumor, reduction in metastasis, complete remission, partial remission, pathologic complete response, or stable disease. In some embodiments, a comparable clinical benefit rate (CBR=CR+PR+SD≧6 months) is obtained with treatment of the PARP inhibitor as compared to treatment with an anti-tumor agent. In some embodiments, the improvement of clinical benefit rate is at least about 30% over treatment with an anti-tumor agent alone. In some embodiments, the PARP inhibitor is 4-iodo-3-nitrobenzamide or a metabolite thereof. In some embodiments, the PARP inhibitor is of Formula (IIa) or a metabolite thereof:

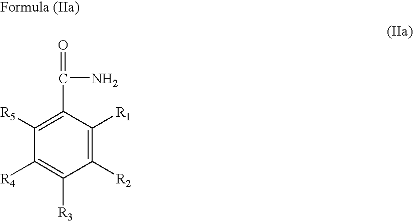

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

In some embodiments, the uterine cancer is a metastatic uterine cancer. In some embodiments, the uterine cancer is an endometrial cancer. In some embodiments, the uterine cancer is recurrent, advanced, or persistent. In some embodiments, the ovarian cancer is a metastatic ovarian cancer. In some embodiments, the ovarian cancer is deficient in homologous recombination DNA repair. In some embodiments, the uterine cancer is deficient in homologous recombination DNA repair. In some embodiments, the uterine cancer is BRCA deficient. In some embodiments, the ovarian cancer is BRCA deficient. In some embodiments, the BRCA-deficiency is a BRCA1-deficiency, or a BRCA2-deficiency, or both BRCA1 and BRCA2-deficiency. In some embodiments, the treatment further comprises (a) establishing a treatment cycle of about 10 to about 30 days in length; and (b) on from 1 to 10 separate days of the cycle, administering to the patient about 1 mg/kg to about 100 mg/kg of 4-iodo-3-nitrobenzamide, or a molar equivalent of a metabolite thereof. In some embodiments, the 4-iodo-3-nitrobenzamide or metabolite thereof is administered orally, or as a parenteral injection or infusion, or inhalation. In some embodiments, the method further comprises administering to the patient a PARP inhibitor in combination with at least one anti-tumor agent. In some embodiments, the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof. In some embodiments, the anti-tumor agent is citabine, capecitabine, valopicitabine or gemcitabine. In some embodiments, the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871. In some embodiments, the method further comprises administering to the patient a PARP inhibitor in combination with more than one anti-tumor agent. In some embodiments, the anti-tumor agent is administered prior to, concomitant with or subsequent to administering the PARP inhibitor. In some embodiments, the method further comprises surgery, radiation therapy, chemotherapy, gene therapy, DNA therapy, adjuvant therapy, neoadjuvant therapy, viral therapy, RNA therapy, immunotherapy, nanotherapy or a combination thereof. In some embodiments, the sample is a tissue or bodily fluid sample. In some embodiments, the sample is a tumor sample, a blood sample, a blood plasma sample, a peritoneal fluid sample, an exudate or an effusion.

In another aspect, the present invention provides a method of treating uterine cancer or ovarian cancer in a patient, comprising administering to the patient a combination of at least one PARP inhibitor and at least one anti-tumor agent. In another aspect, the present invention provides a method of treating ovarian cancer or uterine cancer in a patient in need of such treatment, comprising: (a) obtaining a sample from the patient; (b) testing the sample to determine whether the patient is BRCA deficient; (c) if the testing indicates that the patient is BRCA-deficient, treating the patient with at least one PARP inhibitor and at least one anti-tumor agent. In another aspect, the present invention provides a method of treating uterine cancer or ovarian cancer in a patient, comprising: (a) obtaining a sample from the patient; (b) testing the sample to determine a level of PARP expression in the sample; (c) determining whether the PARP expression exceeds a predetermined level, and if so, administering to the patient at least one PARP inhibitor and at least one anti-tumor agent.

In practicing any of the subject methods disclosed herein, in some embodiments, at least one therapeutic effect is obtained, said at least one therapeutic effect being reduction in size of a uterine tumor or an ovarian tumor, reduction in metastasis, complete remission, partial remission, pathologic complete response, or stable disease. In some embodiments, an improvement of clinical benefit rate (CBR=CR+PR+SD≧6 months) is obtained as compared to treatment with the anti-tumor agent but without the PARP inhibitor. In some embodiments, the improvement of clinical benefit rate is at least about 60%. In some embodiments, the uterine cancer is a metastatic uterine cancer. In some embodiments, the uterine cancer is an endometrial cancer. In some embodiments, the uterine cancer is recurrent, advanced, or persistent. In some embodiments, the ovarian cancer is a metastatic ovarian cancer. In some embodiments, the ovarian cancer is deficient in homologous recombination DNA repair. In some embodiments, the uterine cancer is deficient in homologous recombination DNA repair. In some embodiments, the uterine cancer is BRCA deficient. In some embodiments, the ovarian cancer is BRCA deficient. In some embodiments, the BRCA-deficiency is a BRCA1-deficiency, or BRCA2-deficiency, or both BRCA1 and BRCA2-deficiency. In some embodiments, the PARP inhibitor is a benzamide or a metabolite thereof. In some embodiments, the PARP inhibitor is 4-iodo-3-nitrobenzamide or a metabolite thereof. In some embodiments, the PARP inhibitor is of Formula (IIa) or a metabolite thereof:

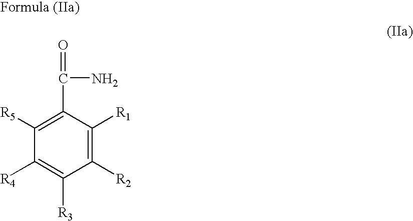

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

In some embodiments, the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof. In some embodiments, the anti-tumor agent is citabine, capecitabine, valopicitabine or gemcitabine. In some embodiments, the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871. In some embodiments, the method further comprises surgery, radiation therapy, chemotherapy, gene therapy, DNA therapy, adjuvant therapy, neoadjuvant therapy, viral therapy, RNA therapy, immunotherapy, nanotherapy or a combination thereof. In some embodiments, the method further comprises selecting a treatment cycle of at least 11 days and: (a) on from 1 to 5 separate days of the cycle, administering to the patient about 100 to about 2000 mg/m² of paclitaxel; (b) on from 1 to 5 separate days of the cycle, administering to the patient about 10-400 mg/m² of carboplatin; and (c) on from 1 to 10 separate days of the cycle, administering to the patient about 1-100 mg/kg of 4-iodo-3-nitrobenzamide. In some embodiments, paclitaxel is administered as an intravenous infusion. In some embodiments, carboplatin is administered as an intravenous infusion. In some embodiments, 4-iodo-3-nitrobenzamide is administered orally or as a parenteral injection or infusion, or inhalation. In some embodiments, the sample is a tissue or bodily fluid sample. In some embodiments, the sample is a tumor sample, a blood sample, a blood plasma sample, a peritoneal fluid sample, an exudate or an effusion.

### INCORPORATION BY REFERENCE

All publications and patent applications mentioned in this specification are herein incorporated by reference to the same extent as if each individual publication or patent application is specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows upregulation of PARP1 gene expression in human primary cancers. Horizontal line, median PARP1 expression; box, interquartile range; bars, standard deviation.

FIG. 2 shows inhibition of PARP by 4-iodo-3-nitrobenzamide in OVCAR-3 xenograft model in SCID mice.

FIG. 3 shows Kaplan-Meier plot of 4-iodo-3-nitrobenzamide in OVCAR-3 ovarian carcinoma tumor model.

FIG. 4 shows tumor response after 4 cycles of BA treatment in combination with topotecan in a patient with ovarian cancer.

FIG. 5 shows PARP inhibition in peripheral mononuclear blood cells (PMBCs) from patients receiving 4-iodo-3-nitrobenzamide.

FIG. 6 shows that BA inhibits proliferation of cervical adenocarcinoma Hela cells.

### DETAILED DESCRIPTION

### Ovarian Cancer Treatment

Ovarian cancer, which ranks fifth in cancer deaths among women, is difficult to detect in its early stages. Approximately only about 20 percent of ovarian cancers are found before tumor growth has spread into adjacent tissues. Three basic types of ovarian tumors exist, including epithelial tumors, germ cell tumors and stromal cell tumors.

A significant risk factor for ovarian cancer includes inherited mutations in BRCA1 or BRCA2 genes. These genes are originally identified in families with multiple cases of breast cancer, but have been associated with approximately 5 to 10 percent of ovarian cancers.

Surgery, immunotherapy, chemotherapy, hormone therapy, radiation therapy, or a combination thereof are some possible treatments available for ovarian cancer. Some possible surgical procedures include debulking, and a unilateral or bilateral oophorectomy and/or a unilateral or bilateral salpigectomy. Anti-cancer drugs that have also been used include cyclophosphamide, etoposide, altretamine, and ifosfamide. Hormone therapy with the drug tamoxifen is also used to shrink ovarian tumors. Radiation therapy optionally includes external beam radiation therapy and/or brachytherapy.

Some embodiments described herein provide a method of treating ovarian cancer in a patient, comprising administering to the patient at least one PARP inhibitor. In some embodiments, at least one therapeutic effect is obtained, said at least one therapeutic effect being reduction in size of an ovarian tumor, reduction in metastasis, complete remission, partial remission, pathologic complete response, or stable disease. In some embodiments, an improvement of clinical benefit rate (CBR=CR+PR+SD≧6 months) is obtained as compared to treatment without the PARP inhibitor. In some embodiments, the improvement of clinical benefit rate is at least about 30%. In some embodiments, the PARP inhibitor is a PARP-1 inhibitor. In other embodiments, the PARP-1 inhibitor is a benzamide or a metabolite thereof. In some embodiments, the benzamide is 4-iodo-3-nitrobenzamide or a metabolite thereof. In some embodiments, the ovarian cancer is a metastatic ovarian cancer. In some embodiments, a deficiency in a BRCA gene is detected in the ovarian cancer patient. In some embodiments, the BRCA gene is BRCA 1. In other embodiments, the BRCA gene is BRCA-2. In yet other embodiments, the BRCA gene is BRCA-1 and BRCA-2. In other embodiments, the deficiency is a genetic defect in the BRCA gene. In some embodiments, the genetic defect is a mutation, insertion, substitution, duplication or deletion of the BRCA gene.

In some embodiments, the methods for treating ovarian cancer further comprise administering a PARP inhibitor in combination with an anti-tumor agent. In some embodiments, the anti-tumor agent is an antitumor alkylating agent, antitumor antimetabolite, antitumor antibiotics, anti-tumor viral agent, plant-derived antitumor agent, antitumor platinum complex, antitumor campthotecin derivative, antitumor tyrosine kinase inhibitor, monoclonal antibody, interferon, biological response modifier, hormonal anti-tumor agent, angiogenesis inhibitor, differentiating agent, or other agent that exhibits anti-tumor activities, or a pharmaceutically acceptable salt thereof. In some embodiments, the platinum complex is cisplatin, carboplatin, oxaplatin or oxaliplatin. In some embodiments, the antimetabolite is citabine, capecitabine, gemcitabine or valopicitabine. In some embodiments, the methods further comprise administering to the patient a PARP inhibitor in combination with more than one anti-tumor agent. In some embodiments, the anti-tumor agent is administered prior to, concomitant with or subsequent to administering the PARP inhibitor. In some embodiments, the anti-tumor agent is an anti-angiogenic agent, such as Avastin or a receptor tyrosine kinase inhibitor including but not limited to Sutent, Nexavar, Recentin, ABT-869, and Axitinib. In some embodiments, the anti-tumor agent is a topoisomerase inhibitor including but not limited to irinotecan, topotecan, or camptothecin. In some embodiments, the anti-tumor agent is a taxane including but not limited to paclitaxel, docetaxel and Abraxane. In some embodiments, the anti-tumor agent is an agent targeting Her-2, e.g. Herceptin or lapatinib. In some embodiments, the anti-tumor agent is a hormone analog, for example, progesterone. In some embodiments, the anti-tumor agent is tamoxifen, a steroidal aromatase inhibitor, a non-steroidal aromatase inhibitor, or Fulvestrant. In some embodiments, the anti-tumor agent is an agent targeting a growth factor receptor. In some embodiments, such agent is an inhibitor of epidermal growth factor receptor (EGFR) including but not limited to Cetuximab and Panitumimab. In some embodiments, the agent targeting a growth factor receptor is an inhibitor of insulin-like growth factor 1 (IGF-1) receptor (IGF1R) such as CP-751871. In other embodiments, the method further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

In some embodiments, the treatment comprises a treatment cycle of at least 11 days, i.e. about 11 to about 30 days in length, wherein on from 1 to 10 separate days of the cycle, the patient receives about 1 to about 100 mg/kg of 4-iodo-3-nitrobenzamide or a molar equivalent of a metabolite thereof. In some embodiments, on from 1 to 10 separate days of the cycle, the patient receives about 1 to about 50 mg/kg of 4-iodo-3-nitrobenzamide or a molar equivalent of a metabolite thereof. In some embodiments, on from 1 to 10 separate days of the cycle, the patient receives about 1, 2, 3, 4, 6, 8 or 10, 12, 14, 16, 18 or 20 mg/kg of 4-iodo-3-nitrobenzamide.

Some embodiment described herein provide a method of treating ovarian cancer in a patient having a deficiency in a BRCA gene, comprising during a 21 day treatment cycle on days 1, 4, 8 and 11 of the cycle, administering to the patient about 10 to about 100 mg/kg of 4-iodo-3-nitrobenzamide or a molar equivalent of a metabolite thereof. In some embodiments, the 4-iodo-3-nitrobenzamide is administered orally or as a parenteral injection or infusion, or inhalation.

Some embodiments described herein provide a method of treating ovarian cancer in a patient having a deficiency in a BRCA gene, comprising: (a) establishing a treatment cycle of about 10 to about 30 days in length; (b) on from 1 to 10 separate days of the cycle, administering to the patient about 1 mg/kg to about 50 mg/kg of 4-iodo-3-nitrobenzamide, or a molar equivalent of a metabolite thereof. In some embodiments, the 4-iodo-3-nitrobenzamide is administered orally or as a parenteral injection or infusion, or inhalation.

Some embodiments provided herein include a method of treating ovarian cancer in a patient in need of such treatment, comprising: (a) obtaining a sample from the patient; (b) testing the sample to determine if there is a deficiency in a BRCA gene; (c) if the testing indicates that the patient has a deficiency in a BRCA gene, treating the patient with at least one PARP inhibitor; and (d) if the testing does not indicate that the patient has a deficiency in a BRCA gene, selecting a different treatment option. In some embodiments, at least one therapeutic effect is obtained, said at least one therapeutic effect being reduction in size of an ovarian tumor, reduction in metastasis, complete remission, partial remission, pathologic complete response, or stable disease. In some embodiments, an improvement of clinical benefit rate (CBR=CR+PR+SD≧6 months) is obtained as compared to treatment without the PARP inhibitor. In some embodiments, the clinical benefit rate is at least about 30%. In some embodiments, the PARP inhibitor is a PARP-1 inhibitor. In other embodiments, the PARP-1 inhibitor is a benzamide or a metabolite thereof. In some embodiments, the benzamide is 4-iodo-3-nitrobenzamide or a metabolite thereof. In some embodiments, the sample is a tissue or bodily fluid sample. In some embodiments, the sample is a tumor sample, a blood sample, a blood plasma sample, a peritoneal fluid sample, an exudate or an effusion. In some embodiments, the ovarian cancer is a metastatic ovarian cancer. In some embodiments, the BRCA gene is BRCA-1. In other embodiments, the BRCA gene is BRCA-2. In some embodiments, the BRCA gene is BRCA-1 and BRCA-2. In other embodiments, the deficiency is a genetic defect in the BRCA gene. In some embodiments, the genetic defect is a mutation, insertion, substitution, duplication or deletion of the BRCA gene.

Some embodiments provide a method of treating ovarian cancer in a patient, comprising: (a) testing a sample from the patient for PARP expression; and (b) if the PARP expression exceeds a predetermined level, administering to the patient at least one PARP inhibitor. In some embodiments, at least one therapeutic effect is obtained, said at least one therapeutic effect being reduction in size of an ovarian tumor, reduction in metastasis, complete remission, partial remission, pathologic complete response, or stable disease. In some embodiments, an improvement of clinical benefit rate (CBR=CR+PR+SD≧6 months) is obtained as compared to treatment without the PARP inhibitor. In some embodiments, the improvement of clinical benefit rate is at least about 30%. In some embodiments, the PARP inhibitor is a PARP-1 inhibitor. In other embodiments, the PARP-1 inhibitor is a benzamide or a metabolite thereof. In some embodiments, the benzamide is 4-iodo-3-nitrobenzamide or a metabolite thereof. In some embodiments, the ovarian cancer is a metastatic ovarian cancer.

### Uterine Cancer and Endometrial Cancer Treatment

Malignant uterine neoplasms containing both carcinomatous and sarcomatous elements are designated in the World Health Organization (WHO) classification of uterine neoplasms as carcinosarcomas. An alternative designation is malignant mixed Mullerian tumor (MMMT). Most uterine carcinosarcomas are monoclonal, with the carcinomatous element being the key element and the sarcomatous component derived from the carcinoma or from a stem cell that undergoes divergent differentiation (ie, metaplastic carcinomas). The sarcomatous component is either homologous (composed of tissues normally found in the uterus) or heterologous (containing tissues not normally found in the uterus, most commonly malignant cartilage or skeletal muscle).

Previous studies investigating a number of single agents in carcinosarcoma of the uterus have reported the following response rates: etoposide (6.5%); doxorubicin (9.8%); cisplatin (18%); ifosfamide (32.2%); paclitaxel (18.2%); and topotecan (10%). Thus the three most active agents discovered to date include cisplatin, ifosfamide, and paclitaxel. A randomized phase III trial comparing ifosfamide to ifosfamide plus cisplatin showed an increased response rate (36% vs. 54%), a slight improvement in median progression-free survival (4 vs. 6 months, p=0.02), but no improvement in median survival (7.6 vs. 9.4 months, p=0.07). A second randomized trial evaluated the role of paclitaxel. In this study, patients are randomized to receive ifosfamide versus the combination of ifosfamide plus paclitaxel and showed an increased response rate (29% vs. 45%), improvement in median progression-free survival (3.6 vs. 5.8 months, p=0.03), and improvement in median survival (8.4 vs. 13.5 months, p=0.03). The use of ifosfamide is cumbersome and results in significant toxicity.

In a highly related disease, endometrial carcinoma, there have been several randomized studies addressing the issue of optimal therapy. These studies have focused on three active agents identified in phase II trials: doxorubicin, platinum agents, and paclitaxel. In one study, 281 women are randomized to doxorubicin alone (60 mg/m²) versus doxorubicin (60 mg/m²) plus cisplatin (50 mg/m²) (AP). There is a statistically significant advantage to combination therapy with regard to response rate (RR) (25% versus 42%; p=0.004) and PFS (3.8 vs 5.7 months; HR 0.74 [95% CI 0.58, 0.94; p=0.14), although no difference in OS is observed (9 vs 9.2 months). Paclitaxel had significant single agent activity with a response rate of 36% in advanced or recurrent endometrial cancer. Thus 317 patients are randomized to paclitaxel and doxorubicin or the standard arm. This trial failed to demonstrate a significant difference in RR, PFS, or OS between the two arms, and AP remained the standard of care. However, since both platinum and paclitaxel had demonstrated high single agent activity, there is as strong interest in including paclitaxel and cisplatin in a front-line regimen for advanced and recurrent endometrial cancer. Subsequently, another study randomized 263 patients to AP versus TAP: doxorubicin (45 mg/m²) and cisplatin (50 mg/m²) on day 1, followed by paclitaxel (160 mg/m² IV over 3 hours) on day 2 (with G-CSF support). TAP is superior to AP in terms of ORR (57% vs 34%; p<0.01), median PFS (8.3 vs 5.3 months; p<0.01) and OS with a median of 15.3 (TAP) versus 12.3 months (AP) (p=0.037). This improved efficacy, however, came at the cost of increased toxicity.

### Uterine Tumors

Uterine tumors consist of the group of neoplasm that can be localized at the corpus, isthmus (the transition between the endocervix and uterine corpus) and cervix. The fallopian tubes and uterine ligaments may also undergo tumor tranformation. Uterine tumors may affect the endometrium, muscles or other supporting tissue. Uterine tumors are histologically and biologically different and can be divided into several types. Uterine tumors may be histologically typed according to several classification systems. Those used most frequently are based on the WHO (World Health Organization) International Histological Classification of Tumours and on the ISGYP (International Society of Gynecological Pathologists). The most widely-accepted staging system is the FIGO (International Federation of Gynecology and Obstetrics) one.

Classification

According to WHO recommendations, the main UTERINE CERVIX categories are: Epithelial tumors; Mesemchymal tumors; Mixed epithelial and mesenchymal tumors; and Secondary tumors. The main uterine corpus categories, once again according to WHO recommendations, are: epithelial tumors, mesemchymal tumors, mixed epithelial and mesenchymal tumors, trophoblastic tumors, and secondary tumors. Uterine cancer is the most common, specifically endometrial cancer of the uterine corpus.

### Uterine Corpus Neoplasia

The most common uterine corpus malignancy is the endometrial carcinoma (approximately 95%); sarcomas represent only 4% and heterologous tumors such as rhabdomyosarcomas, osteosarcomas and chondrosarcomas the remaining 1%.

Endometrial carcinoma has several subtypes that based on origin, differentiation, genetic background and clinical outcome. Endometrial carcinoma is defined as an epithelial tumor, usually with glandular differentiation, arising in the endometrium and which has the potential to invade the myometrium and spread to distant sites. Endometrial carcinoma can be classified as endometrioid adenocarcinoma, serous carcinoma, clear cell carcinoma, mucinous carcinoma, serous carcinoma, mixed types of carcinoma, and undifferentiated carcinoma. Endometrial carcinoma is an heterogeneous entity, comprising of: type I: endometrioid carcinoma: pre- and perimenopausal, estrogen dependent, associated to endometrial hyperplasia, low grade, indolent behaviour, representing about 80%° of the cases; type II: serous carcinoma: post-menopausal, estrogen independent, associated to atrophic endometrium, high grade, aggressive behaviour, representing about 10% of the cases. Among other histologic types, type I includes mucinous and secretory carcinomas, whereas type II includes clear-cell carcinomas and adenosquamous carcinomas (Gurpide E, J Natl Cancer Inst 1991; 83: 405-416; Blaustein's Pathology of the Female Genital Tract, Kurman R. J. 4th ed. Springer-Verlag. New-York 1994).

### Uterine Cervix Neoplasia

Worldwide, invasive cervical cancer is the second most common female malignancy after breast cancer, with 500,000 new cases diagnosed each year. Uterine cervix cancers has several subtypes such as epithelial neoplasia and mesenchymal neoplasia.

Etiology

Carcinomas of the uterine cervix are thought to arise from precursor lesions, and different subtypes of human papilloma virus (HPV) are major etiological factors in disease pathogenesis.

Heterogenity of uterine tumors provide a challenge to find and optimize the therapy to treat and cure these types of cancers and chemotherapeutic agent that are efficacious for other cancers are not efficacious for uterin tumors such as endometrial cancer. One of the examples could be Tamoxifen. Tamoxifen, a selective estrogen receptor (ER) modulator, is the most widely prescribed hormonal therapy treatment for breast cancer. Despite the benefits of tamoxifen therapy, almost all tamoxifen-responsive breast cancer patients develop resistance to therapy. Despite some benefits of tamoxifen therapy, almost all tamoxifen-responsive breast cancer patients develop resistance to therapy. In addition, tamoxifen displays estrogen-like effects in the endometrium increasing the incidence of endometrial cancer (Fisher B, Costantino J P, Redmond C K, et al. J Natl Cancer Inst 1994; 86:527-37; Shah Y M, et. al. Mol Cancer Ther. 2005 August; 4(8):1239-49).

In patients with persistent or recurrent nonsquamous cell carcinoma of the cervix, the study was undertaken by Gynecologic Oncology Group to estimate the antitumor activity of tamoxifen (L. R. Bigler, J. et. al. (2004) International Journal of Gynecological Cancer 14 (5), 871-874). Tamoxifen citrate is administered at a dose of 10 mg per orally twice a day until disease progression or unacceptable side effects prevented further therapy. A total of 34 patients (median age: 49 years) are registered to this trial; two are declared ineligible. Thirty-two patients are evaluable for adverse effects and 27 are evaluable for response. There are only six grades 3 and 4 adverse effects reported: leukopenia (in one patient), anemia (in two), emesis (in one), gastrointestinal distress (in one), and neuropathy (in one). The objective response rate is 11.1%, with one complete and two partial responses. In conclusion, tamoxifen appears to have minimal activity in nonsquamous cell carcinoma of the cervix.

Accordingly, some embodiments described herein provide a method of treating uterine cancer or endometrial cancer in a patient, comprising administering to the patient at least one PARP inhibitor. In some embodiments, at least one therapeutic effect is obtained, said at least one therapeutic effect being reduction in size of a uterine tumor, reduction in metastasis, complete remission, partial remission, pathologic complete response, or stable disease. In some embodiments, an improvement of clinical benefit rate (CBR=CR+PR+SD≧6 months) is obtained as compared to treatment without the PARP inhibitor. In some embodiments, the improvement of clinical benefit rate is at least about 30%. In some embodiments, the PARP inhibitor is a PARP-1 inhibitor. In other embodiments, the PARP-1 inhibitor is a benzamide or a metabolite thereof. In some embodiments, the benzamide is 4-iodo-3-nitrobenzamide or a metabolite thereof. In some embodiments, the uterine cancer is a metastatic uterine cancer. In some embodiments, the uterine cancer is recurrent, advanced or persistent.

In some embodiments, the methods for treating uterine cancer or endometrial cancer further comprise administering a PARP inhibitor in combination with an anti-tumor agent. In some embodiments, the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, or other agent that exhibits anti-tumor activities, or a pharmaceutically acceptable salt thereof. In some embodiments, the platinum complex is cisplatin, carboplatin, oxaplatin or oxaliplatin. In some embodiments, the antimetabolite is citabine, capecitabine, gemcitabine or valopicitabine. In some embodiments, the methods further comprise administering to the patient a PARP inhibitor in combination with more than one anti-tumor agent. In some embodiments, the anti-tumor agent is administered prior to, concomitant with or subsequent to administering the PARP inhibitor. In some embodiments, the anti-tumor agent is an anti-angiogenic agent, such as Avastin or a receptor tyrosine kinase inhibitor including but not limited to Sutent, Nexavar, Recentin, ABT-869, and Axitinib. In some embodiments, the anti-tumor agent is a topoisomerase inhibitor including but not limited to irinotecan, topotecan, or camptothecin. In some embodiments, the anti-tumor agent is a taxane including but not limited to paclitaxel, docetaxel and Abraxane. In some embodiments, the anti-tumor agent is an agent targeting Her-2, e.g. Herceptin or lapatinib. In some embodiments, the anti-tumor agent is a hormone analog, for example, progesterone. In some embodiments, the anti-tumor agent is tamoxifen, a steroidal aromatase inhibitor, a non-steroidal aromatase inhibitor, or Fulvestrant. In some embodiments, the anti-tumor agent is an agent targeting a growth factor receptor. In some embodiments, such agent is an inhibitor of epidermal growth factor receptor (EGFR) including but not limited to Cetuximab and Panitumimab. In some embodiments, the agent targeting a growth factor receptor is an inhibitor of insulin-like growth factor 1 (IGF-1) receptor (IGF1R) such as CP-751871. In other embodiments, the method further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

In some embodiments, the treatment comprises a treatment cycle of at least 11 days, i.e. about 11 to about 30 days in length, wherein on from 1 to 10 separate days of the cycle, the patient receives about 1 to about 100 mg/kg of 4-iodo-3-nitrobenzamide or a molar equivalent of a metabolite thereof. In some embodiments, on from 1 to 10 separate days of the cycle, the patient receives about 1 to about 50 mg/kg of 4-iodo-3-nitrobenzamide or a molar equivalent of a metabolite thereof. In some embodiments, on from 1 to 10 separate days of the cycle, the patient receives about 1, 2, 3, 4, 6, 8 or 10, 12, 14, 16, 18 or 20 mg/kg of 4-iodo-3-nitrobenzamide.

Some embodiment described herein provide a method of treating uterine cancer or endometrial cancer in a patient, comprising during a 21 day treatment cycle on days 1, 4, 8 and 11 of the cycle, administering to the patient about 1 to about 100 mg/kg of 4-iodo-3-nitrobenzamide or a molar equivalent of a metabolite thereof. In some embodiments, the 4-iodo-3-nitrobenzamide is administered orally or as a parenteral injection or infusion, or inhalation.

Some embodiments described herein provide a method of treating uterine cancer or endometrial cancer in a patient, comprising: (a) establishing a treatment cycle of about 10 to about 30 days in length; (b) on from 1 to 10 separate days of the cycle, administering to the patient about 1 mg/kg to about 100 mg/kg of 4-iodo-3-nitrobenzamide, or a molar equivalent of a metabolite thereof. In some embodiments, the 4-iodo-3-nitrobenzamide is administered orally or as a parenteral injection or infusion, or inhalation.

Some embodiments provided herein include a method of treating uterine cancer in a patient in need of such treatment, comprising: (a) obtaining a sample from the patient; (b) determining if the uterine cancer is recurrent, persistent or advanced; (c) if the testing indicates that the uterine cancer is recurrent, persistent or advanced, treating the patient with at least one PARP inhibitor; and (d) if the testing does not indicate that the patient has a uterine cancer that is recurrent, persistent or advanced, selecting a different treatment option. In some embodiments, at least one therapeutic effect is obtained, said at least one therapeutic effect being reduction in size of a uterine tumor, reduction in metastasis, complete remission, partial remission, pathologic complete response, or stable disease. In some embodiments, an improvement of clinical benefit rate (CBR=CR+PR+SD≧6 months) is obtained as compared to treatment without the PARP inhibitor. In some embodiments, the clinical benefit rate is at least about 30%. In some embodiments, the PARP inhibitor is a PARP-1 inhibitor. In other embodiments, the PARP-1 inhibitor is a benzamide or a metabolite thereof. In some embodiments, the benzamide is 4-iodo-3-nitrobenzamide or a metabolite thereof. In some embodiments, the sample is a tissue or bodily fluid sample. In some embodiments, the sample is a tumor sample, a blood sample, a blood plasma sample, a peritoneal fluid sample, an exudate or an effusion. In some embodiments, the uterine cancer is a metastatic uterine cancer.

Some embodiments provide a method of treating uterine cancer, endometrial cancer, or ovarian cancer in a patient, comprising: (a) testing a sample from the patient for PARP expression; and (b) if the PARP expression exceeds a predetermined level, administering to the patient at least one PARP inhibitor. In some embodiments, at least one therapeutic effect is obtained, said at least one therapeutic effect being reduction in size of a uterine tumor, reduction in metastasis, complete remission, partial remission, pathologic complete response, or stable disease. In some embodiments, an improvement of clinical benefit rate (CBR=CR+PR+SD≧6 months) is obtained as compared to treatment without the PARP inhibitor. In some embodiments, the improvement of clinical benefit rate is at least about 30%. In some embodiments, the PARP inhibitor is a PARP-1 inhibitor. In other embodiments, the PARP-1 inhibitor is a benzamide or a metabolite thereof. In some embodiments, the benzamide is 4-iodo-3-nitrobenzamide or a metabolite thereof. In some embodiments, the uterine cancer is a metastatic uterine cancer. In some embodiments, the ovarian cancer is a metastatic ovarian cancer.

Thus, embodiments provided herein comprise treating a patient with at least one of which is a PARP inhibitor, wherein the PARP inhibitor is optionally a PARP-1 inhibitor. In some embodiments, one or more of these substances may be capable of being present in a variety of physical forms—e.g. free base, salts (especially pharmaceutically acceptable salts), hydrates, polymorphs, solvates, etc. Unless otherwise qualified herein, use of a chemical name is intended to encompass all physical forms of the named chemical. For example, recitation of 4-iodo-3-nitrobenzamide, without further qualification, is intended to generically encompass the free base as well as all pharmaceutically acceptable salts, polymorphs, hydrates, etc. Where it is intended to limit the disclosure or claims to a particular physical form of a compound, this will be clear from the context of the passage or claim in which the reference to the compound appears.

In some embodiments, the disclosure herein provides a method of treating uterine cancer, endometrial cancer, or ovarian cancer in a patient, comprising administering to the patient a combination of at least one anti-tumor agent and at least one PARP inhibitor. In some embodiments, at least one therapeutic effect is obtained, said at least one therapeutic effect being reduction in size of a tumor, reduction in metastasis, complete remission, partial remission, pathologic complete response, or stable disease. In some embodiments, the PARP inhibitor is a benzamide or a metabolite thereof. In some embodiments, the benzamide is 4-iodo-3-nitrobenzamide or a metabolite thereof. In some embodiments, the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, or other agent that exhibits anti-tumor activities, or a pharmaceutically acceptable salt thereof. In some embodiments, the platinum complex is selected from the group consisting of cisplatin, carboplatin, oxaplatin and oxaliplatin. In some embodiments, the platinum complex is carboplatin. In some embodiments, the taxane is paclitaxel or docetaxel. In some embodiments, the taxane is paclitaxel. In some embodiments, the anti-tumor agent is an anti-angiogenic agent, such as Avastin or a receptor tyrosine kinase inhibitor including but not limited to Sutent, Nexavar, Recentin, ABT-869, and Axitinib. In some embodiments, the anti-tumor agent is a topoisomerase inhibitor including but not limited to irinotecan, topotecan, or camptothecin. In some embodiments, the anti-tumor agent is a taxane including but not limited to paclitaxel, docetaxel and Abraxane. In some embodiments, the anti-tumor agent is an agent targeting Her-2, e.g. Herceptin or lapatinib. In some embodiments, the anti-tumor agent is a hormone analog, for example, progesterone. In some embodiments, the anti-tumor agent is tamoxifen, a steroidal aromatase inhibitor, a non-steroidal aromatase inhibitor, or Fulvestrant. In some embodiments, the anti-tumor agent is an agent targeting a growth factor receptor. In some embodiments, such agent is an inhibitor of epidermal growth factor receptor (EGFR) including but not limited to Cetuximab and Panitumimab. In some embodiments, the agent targeting a growth factor receptor is an inhibitor of insulin-like growth factor 1 (IGF-1) receptor (IGF1R) such as CP-751871. In some embodiments, the cancer is a uterine cancer. In some embodiments, the cancer is advanced uterine carcinosarcoma, persistent uterine carcinosarcoma or recurrent uterine carcinosarcoma. In some embodiments, the cancer is endometrial cancer. In some embodiments, the cancer is ovarian cancer. In some embodiments, the cancer is a metastatic ovarian cancer or uterine cancer. In some embodiments, the method comprises selecting a treatment cycle of at least 11 days and: (a) on day 1 of the cycle, administering to the patient about 10-200 mg/m² of paclitaxel; (b) on day 1 of the cycle, administering to the patient about 10-400 mg/m² carboplatin; and (c) on day 1 and twice weekly throughout the cycle, administering to the patient about 1-100 mg/kg of 4-iodo-3-nitrobenzamide or a molar equivalent of a metabolite thereof.

In some embodiments, the disclosure provides a method of treating uterine cancer, endometrial cancer, or ovarian cancer in a patient, comprising: (a) obtaining a sample from the patient; (b) testing the sample to determine a level of PARP expression in the sample; (c) determining whether the PARP expression exceeds a predetermined level, and if so, administering to the patient at least one taxane, at least one platinum complex and at least one PARP inhibitor. In some embodiments, the method further comprises optionally selecting a different treatment option if the PARP expression in the sample does not exceed the predetermined level. In some embodiments, the method optionally further comprises selecting a different treatment option if the PARP expression in the sample does not exceed the predetermined level. In some embodiments, the cancer is a uterine cancer. In some embodiments, the cancer is advanced uterine carcinosarcoma, persistent uterine carcinosarcoma or recurrent uterine carcinosarcoma. In some embodiments, the cancer is an endometrial cancer. In some embodiments, the cancer is an ovarian cancer. In some embodiments, the cancer is a metastatic ovarian cancer. In some embodiments, the taxane is cisplatin, carboplatin, oxaplatin or oxaliplatin.

In some embodiments, the taxane is paclitaxel. In some embodiments, the platinum complex is cisplatin or carboplatin. In some embodiments, the platinum complex is carboplatin. In some embodiments, the PARP inhibitor is a benzamide or a metabolite thereof. In some embodiments, the PARP inhibitor is 4-iodo-3-nitrobenzamide. In some embodiments, the sample is a tissue sample or a bodily fluid sample.

In some embodiments, the present disclosure provides a method of treating uterine cancer, endometrial cancer, or ovarian cancer in a patient, comprising during a 21 day treatment cycle: (a) on day 1 of the cycle, administering to the patient about 750 mg/m² of paclitaxel; (b) on day 1 of the cycle, administering to the patient about 10-400 mg/m² of carboplatin; and (c) on day 1 of the cycle, and twice weekly thereafter, administering to the patient about 1-100 mg/kg of 4-iodo-3-nitrobenzamide. In some embodiments, the paclitaxel is administered as an intravenous infusion. In some embodiments, the carboplatin is administered as an intravenous infusion. In some embodiments, the 4-iodo-3-nitrobenzamide is administered orally or as a parenteral injection or infusion, or inhalation. In some embodiments, the cancer is a uterine cancer selected from advanced uterine carcinosarcoma, persistent uterine carcinosarcoma and recurrent uterine carcinosarcoma. In some embodiments, the cancer is ovarian cancer.

Some embodiments described herein provide a method of treating uterine cancer, endometrial cancer, or ovarian cancer in a patient, comprising: (a) establishing a treatment cycle of about 10 to about 30 days in length; (b) on from 1 to 5 separate days of the cycle, administering to the patient about 100 to about 2000 mg/m² of paclitaxel by intravenous infusion over about 10 to about 300 minutes; (c) on from 1 to 5 separate days of the cycle, administering to the patient about 10-400 mg/m² of carboplatin by intravenous infusion over about 10 to about 300 minutes; and (d) on from 1 to 10 separate days of the cycle, administering to the patient about 1 mg/kg to about 8 mg/kg of 4-iodo-3-nitrobenzamide over about 10 to about 300 minutes.

Some embodiments described herein provide a method of treating uterine cancer in a patient in need of such treatment, comprising: (a) testing a uterine tumor sample from the patient to determine at least one of the following: (i) whether the uterine cancer is advanced; (ii) whether the uterine cancer is persistent; (iii) whether the uterine cancer is recurrent; (b) if the testing indicates that the uterine cancer is advance, persistent or recurrent, treating the patient with a combination of therapeutic agents, wherein the therapeutic agents include at least one anti-tumor agent and at least one PARP inhibitor. In some embodiments, the at least one therapeutic effect is obtained, said at least one therapeutic effect being reduction in size of a uterine tumor, reduction in metastasis, complete remission, partial remission, pathologic complete response, or stable disease. In some embodiments, the PARP inhibitor is a benzamide or a metabolite thereof.

In some embodiments, the benzamide is 4-iodo-3-nitrobenzamide or a metabolite thereof. In some embodiments, the platinum complex is selected from the group consisting of cisplatin, carboplatin, oxaplatin and oxaliplatin. In some embodiments, the platinum complex is carboplatin. In some embodiments, the taxane is paclitaxel or docetaxel. In some embodiments, the taxane is paclitaxel. In some embodiments, the cancer is an advanced carcinosarcoma, a persistent carcinosarcoma or a recurrent carcinosarcoma. In some embodiments, the cancer is an endometrial cancer. In some embodiments, the method comprises treating a patient with at least three chemically distinct substances, one of which is a taxane (e.g. paclitaxel or docetaxel), one of which is a platinum-containing complex (e.g. cisplatin or carboplatin or cisplatin) and one of which is a PARP inhibitor (e.g. BA or a metabolite thereof). In some embodiments, one or more of these substances may be capable of being present in a variety of physical forms—e.g. free base, salts (especially pharmaceutically acceptable salts), hydrates, polymorphs, solvates, or metabolites, etc. Unless otherwise qualified herein, use of a chemical name is intended to encompass all physical forms of the named chemical. For example, recitation of 4-iodo-3-nitrobenzamide, without further qualification, is intended to generically encompass the free base as well as all pharmaceutically acceptable salts, polymorphs, hydrates, and metabolites thereof. Where it is intended to limit the disclosure or claims to a particular physical form of a compound, this will be clear from the context of the passage or claim in which the reference to the compound appears.

The terms “effective amount” or “pharmaceutically effective amount” refer to a sufficient amount of the agent to provide the desired biological, therapeutic, and/or prophylactic result. That result can be reduction and/or alleviation of the signs, symptoms, or causes of a disease, or any other desired alteration of a biological system. For example, an “effective amount” for therapeutic uses is the amount of a nitrobenzamide compound as disclosed herein per se or a composition comprising the nitrobenzamide compound herein required to provide a clinically significant decrease in a disease. An appropriate effective amount in any individual case may be determined by one of ordinary skill in the art using routine experimentation.

By “pharmaceutically acceptable” or “pharmacologically acceptable” is meant a material which is not biologically or otherwise undesirable, i.e., the material may be administered to an individual without causing significant undesirable biological effects or interacting in a deleterious manner with any of the components of the composition in which it is contained.

The term “treating” and its grammatical equivalents as used herein include achieving a therapeutic benefit and/or a prophylactic benefit. By therapeutic benefit is meant eradication or amelioration of the underlying disorder being treated. For example, in a cancer patient, therapeutic benefit includes eradication or amelioration of the underlying cancer. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For prophylactic benefit, a method of the invention may be performed on, or a composition of the invention administered to a patient at risk of developing cancer, or to a patient reporting one or more of the physiological symptoms of such conditions, even though a diagnosis of the condition may not have been made.

### Anti-Tumor Agents

Anti-tumor agents that may be used in the present invention include but are not limited to antitumor alkylating agents, antitumor antimetabolites, antitumor antibiotics, plant-derived antitumor agents, antitumor platinum-complex compounds, antitumor campthotecin derivatives, antitumor tyrosine kinase inhibitors, anti-tumor viral agent, monoclonal antibodies, interferons, biological response modifiers, and other agents that exhibit anti-tumor activities, or a pharmaceutically acceptable salt thereof.

In some embodiments, the anti-tumor agent is an alkylating agent. The term “alkylating agent” herein generally refers to an agent giving an alkyl group in the alkylation reaction in which a hydrogen atom of an organic compound is substituted with an alkyl group. Examples of anti-tumor alkylating agents include but are not limited to nitrogen mustard N-oxide, cyclophosphamide, ifosfamide, melphalan, busulfan, mitobronitol, carboquone, thiotepa, ranimustine, nimustine, temozolomide or carmustine.

In some embodiments, the anti-tumor agent is an antimetabolite. The term “antimetabolite” used herein includes, in a broad sense, substances which disturb normal metabolism and substances which inhibit the electron transfer system to prevent the production of energy-rich intermediates, due to their structural or functional similarities to metabolites that are important for living organisms (such as vitamins, coenzymes, amino acids and saccharides). Examples of antimetabolites that have anti-tumor activities include but are not limited to methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine or pemetrexed disodium, and preferred are 5-fluorouracil, S-1, gemcitabine and the like.

In some embodiments, the anti-tumor agent is an antitumor antibiotic. Examples of antitumor antibiotics include but are not limited to actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus or valrubicin. In some embodiments, the anti-tumor agent is a plant-derived antitumor agent. Examples of plant-derived antitumor agents include but are not limited to vincristine, vinblastine, vindesine, etoposide, sobuzoxane, docetaxel, paclitaxel and vinorelbine, and preferred and docetaxel and paclitaxel.

In some embodiments, the anti-tumor agent is a camptothecin derivative that exhibits anti-tumor activities. Examples of anti-tumor camptothecin derivatives include but are not limited to camptothecin, 10-hydroxycamptothecin, topotecan, irinotecan or 9-aminocamptothecin, with camptothecin, topotecan and irinotecan being preferred. Further, irinotecan is metabolized in vivo and exhibits antitumor effect as SN-38. The action mechanism and the activity of the camptothecin derivatives are believed to be virtually the same as those of camptothecin (e.g., Nitta, et al., Gan to Kagaku Ryoho, 14, 850-857 (1987)).

In some embodiments, the anti-tumor agent is an organoplatinum compound or a platinum coordination compound having antitumor activity. Organoplatinum compound herein refers to a platinum containing compound which provides platinum in ion form. Preferred organoplatinum compounds include but are not limited to cisplatin; cis-diamminediaquoplatinum (II)-ion; chloro(diethylenetriamine)-platinum (II) chloride; dichloro(ethylenediamine)-platinum (II); diammine(1,1-cyclobutanedicarboxylato) platinum (II) (carboplatin); spiroplatin; iproplatin; diammine(2-ethylmalonato)platinum (II); ethylenediaminemalonatoplatinum (II); aqua(1,2-diaminodicyclohexane)sulfatoplatinum (II); aqua(1,2-diaminodicyclohexane)malonatoplatinum (II); (1,2-diaminocyclohexane)malonatoplatinum (II); (4-carboxyphthalato)(1,2-diaminocyclohexane) platinum (II); (1,2-diaminocyclohexane)-(isocitrato)platinum (II); (1,2-diaminocyclohexane)oxalatoplatinum (II); ormaplatin; tetraplatin; carboplatin, nedaplatin and oxaliplatin, and preferred is carboplatin or oxaliplatin. Further, other antitumor organoplatinum compounds mentioned in the specification are known and are commercially available and/or producible by a person having ordinary skill in the art by conventional techniques.

In some embodiments, the anti-tumor agent is an antitumor tyrosine kinase inhibitor. The term “tyrosine kinase inhibitor” herein refers to a chemical substance inhibiting “tyrosine kinase” which transfers a k-phosphate group of ATP to a hydroxyl group of a specific tyrosine in protein. Examples of anti-tumor tyrosine kinase inhibitors include but are not limited to gefitinib, imatinib, erlotinib, Sutent, Nexavar, Recentin, ABT-869, and Axitinib.

In some embodiments, the anti-tumor agent is an antibody or a binding portion of an antibody that exhibits anti-tumor activity. In some embodiments, the anti-tumor agent is a monoclonal antibody. Examples thereof include but are not limited to abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, trastuzumab, or any antibody fragments specific for antigens.

In some embodiments, the anti-tumor agent is an interferon. Such interferon has antitumor activity, and it is a glycoprotein which is produced and secreted by most animal cells upon viral infection. It has not only the effect of inhibiting viral growth but also various immune effector mechanisms including inhibition of growth of cells (in particular, tumor cells) and enhancement of the natural killer cell activity, thus being designated as one type of cytokine. Examples of anti-tumor interferons include but are not limited to interferon α, interferon α-2a, interferon α-2b, interferon β, interferon γ-1a and interferon γ-n1.

In some embodiments, the anti-tumor agent is a biological response modifier. It is generally the generic term for substances or drugs for modifying the defense mechanisms of living organisms or biological responses such as survival, growth or differentiation of tissue cells in order to direct them to be useful for an individual against tumor, infection or other diseases. Examples of the biological response modifier include but are not limited to krestin, lentinan, sizofuran, picibanil and ubenimex.

In some embodiments, the anti-tumor agents include but are not limited to mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

The above-described terms “antitumor alkylating agent”, “antitumor antimetabolite”, “antitumor antibiotic”, “plant-derived antitumor agent”, “antitumor platinum coordination compound”, “antitumor camptothecin derivative”, “antitumor tyrosine kinase inhibitor”, “monoclonal antibody”, “interferon”, “biological response modifier” and “other antitumor agent” are all known and are either commercially available or producible by a person skilled in the art by methods known per se or by well-known or conventional methods. The process for preparation of gefitinib is described, for example, in U.S. Pat. No. 5,770,599; the process for preparation of cetuximab is described, for example, in WO 96/40210; the process for preparation of bevacizumab is described, for example, in WO 94/10202; the process for preparation of oxaliplatin is described, for example, in U.S. Pat. Nos. 5,420,319 and 5,959,133; the process for preparation of gemcitabine is described, for example, in U.S. Pat. Nos. 5,434,254 and 5,223,608; and the process for preparation of camptothecin is described in U.S. Pat. Nos. 5,162,532, 5,247,089, 5,191,082, 5,200,524, 5,243,050 and 5,321,140; the process for preparation of irinotecan is described, for example, in U.S. Pat. No. 4,604,463; the process for preparation of topotecan is described, for example, in U.S. Pat. No. 5,734,056; the process for preparation of temozolomide is described, for example, in JP-B No. 4-5029; and the process for preparation of rituximab is described, for example, in JP-W No. 2-503143.

The above-mentioned antitumor alkylating agents are commercially available, as exemplified by the following: nitrogen mustard N-oxide from Mitsubishi Pharma Corp. as Nitrorin (tradename); cyclophosphamide from Shionogi & Co., Ltd. as Endoxan (tradename); ifosfamide from Shionogi & Co., Ltd. as Ifomide (tradename); melphalan from GlaxoSmithKline Corp. as Alkeran (tradename); busulfan from Takeda Pharmaceutical Co., Ltd. as Mablin (tradename); mitobronitol from Kyorin Pharmaceutical Co., Ltd. as Myebrol (tradename); carboquone from Sankyo Co., Ltd. as Esquinon (tradename); thiotepa from Sumitomo Pharmaceutical Co., Ltd. as Tespamin (tradename); ranimustine from Mitsubishi Pharma Corp. as Cymerin (tradename); nimustine from Sankyo Co., Ltd. as Nidran (tradename); temozolomide from Schering Corp. as Temodar (tradename); and carmustine from Guilford Pharmaceuticals Inc. as Gliadel Wafer (tradename).

The above-mentioned antitumor antimetabolites are commercially available, as exemplified by the following: methotrexate from Takeda Pharmaceutical Co., Ltd. as Methotrexate (tradename); 6-mercaptopurine riboside from Aventis Corp. as Thioinosine (tradename); mercaptopurine from Takeda Pharmaceutical Co., Ltd. as Leukerin (tradename); 5-fluorouracil from Kyowa Hakko Kogyo Co., Ltd. as 5-FU (tradename); tegafur from Taiho Pharmaceutical Co., Ltd. as Futraful (tradename); doxyfluridine from Nippon Roche Co., Ltd. as Furutulon (tradename); carmofur from Yamanouchi Pharmaceutical Co., Ltd. as Yamafur (tradename); cytarabine from Nippon Shinyaku Co., Ltd. as Cylocide (tradename); cytarabine ocfosfate from Nippon Kayaku Co., Ltd. as Strasid (tradename); enocitabine from Asahi Kasei Corp. as Sanrabin (tradename); S-1 from Taiho Pharmaceutical Co., Ltd. as TS-1 (tradename); gemcitabine from Eli Lilly & Co. as Gemzar (tradename); fludarabine from Nippon Schering Co., Ltd. as Fludara (tradename); and pemetrexed disodium from Eli Lilly & Co. as Alimta (tradename).

The above-mentioned antitumor antibiotics are commercially available, as exemplified by the following: actinomycin D from Banyu Pharmaceutical Co., Ltd. as Cosmegen (tradename); doxorubicin from Kyowa Hakko Kogyo Co., Ltd. as adriacin (tradename); daunorubicin from Meiji Seika Kaisha Ltd. as Daunomycin; neocarzinostatin from Yamanouchi Pharmaceutical Co., Ltd. as Neocarzinostatin (tradename); bleomycin from Nippon Kayaku Co., Ltd. as Bleo (tradename); pepromycin from Nippon Kayaku Co, Ltd. as Pepro (tradename); mitomycin C from Kyowa Hakko Kogyo Co., Ltd. as Mitomycin (tradename); aclarubicin from Yamanouchi Pharmaceutical Co., Ltd. as Aclacinon (tradename); pirarubicin from Nippon Kayaku Co., Ltd. as Pinorubicin (tradename); epirubicin from Pharmacia Corp. as Pharmorubicin (tradename); zinostatin stimalamer from Yamanouchi Pharmaceutical Co., Ltd. as Smancs (tradename); idarubicin from Pharmacia Corp. as Idamycin (tradename); sirolimus from Wyeth Corp. as Rapamune (tradename); and valrubicin from Anthra Pharmaceuticals Inc. as Valstar (tradename).

The above-mentioned plant-derived antitumor agents are commercially available, as exemplified by the following: vincristine from Shionogi & Co., Ltd. as Oncovin (tradename); vinblastine from Kyorin Pharmaceutical Co., Ltd. as Vinblastine (tradename); vindesine from Shionogi & Co., Ltd. as Fildesin (tradename); etoposide from Nippon Kayaku Co., Ltd. as Lastet (tradename); sobuzoxane from Zenyaku Kogyo Co., Ltd. as Perazolin (tradename); docetaxel from Aventis Corp. as Taxsotere (tradename); paclitaxel from Bristol-Myers Squibb Co. as Taxol (tradename); and vinorelbine from Kyowa Hakko Kogyo Co., Ltd. as Navelbine (tradename).

The above-mentioned antitumor platinum coordination compounds are commercially available, as exemplified by the following: cisplatin from Nippon Kayaku Co., Ltd. as Randa (tradename); carboplatin from Bristol-Myers Squibb Co. as Paraplatin (tradename); nedaplatin from Shionogi & Co., Ltd. as Aqupla (tradename); and oxaliplatin from Sanofi-Synthelabo Co. as Eloxatin (tradename).

The above-mentioned antitumor camptothecin derivatives are commercially available, as exemplified by the following: irinotecan from Yakult Honsha Co., Ltd. as Campto (tradename); topotecan from GlaxoSmithKline Corp. as Hycamtin (tradename); and camptothecin from Aldrich Chemical Co., Inc., U.S.A.

The above-mentioned antitumor tyrosine kinase inhibitors are commercially available, as exemplified by the following: gefitinib from AstraZeneca Corp. as Iressa (tradename); imatinib from Novartis AG as Gleevec (tradename); and erlotinib from OSI Pharmaceuticals Inc. as Tarceva (tradename).

The above-mentioned monoclonal antibodies are commercially available, as exemplified by the following: cetuximab from Bristol-Myers Squibb Co. as Erbitux (tradename); bevacizumab from Genentech, Inc. as Avastin (tradename); rituximab from Biogen Idec Inc. as Rituxan (tradename); alemtuzumab from Berlex Inc. as Campath (tradename); and trastuzumab from Chugai Pharmaceutical Co., Ltd. as Herceptin (tradename).

The above-mentioned interferons are commercially available, as exemplified by the following: interferon α from Sumitomo Pharmaceutical Co., Ltd. as Sumiferon (tradename); interferon α-2a from Takeda Pharmaceutical Co., Ltd. as Canferon-A (tradename); interferon α-2b from Schering-Plough Corp. as Intron A (tradename); interferon β from Mochida Pharmaceutical Co., Ltd. as IFN.beta. (tradename); interferon γ-1a from Shionogi & Co., Ltd. as Immunomax-γ (tradename); and interferon γ-n1 from Otsuka Pharmaceutical Co., Ltd. as Ogamma (tradename).

The above-mentioned biological response modifiers are commercially available, as exemplified by the following: krestin from Sankyo Co., Ltd. as krestin (tradename); lentinan from Aventis Corp. as Lentinan (tradename); sizofuran from Kaken Seiyaku Co., Ltd. as Sonifuran (tradename); picibanil from Chugai Pharmaceutical Co., Ltd. as Picibanil (tradename); and ubenimex from Nippon Kayaku Co., Ltd. as Bestatin (tradename).

The above-mentioned other antitumor agents are commercially available, as exemplified by the following: mitoxantrone from Wyeth Lederle Japan, Ltd. as Novantrone (tradename); L-asparaginase from Kyowa Hakko Kogyo Co., Ltd. as Leunase (tradename); procarbazine from Nippon Roche Co., Ltd. as Natulan (tradename); dacarbazine from Kyowa Hakko Kogyo Co., Ltd. as Dacarbazine (tradename); hydroxycarbamide from Bristol-Myers Squibb Co. as Hydrea (tradename); pentostatin from Kagaku Oyobi Kessei Ryoho Kenkyusho as Coforin (tradename); tretinoin from Nippon Roche Co., Ltd. As Vesanoid (tradename); alefacept from Biogen Idec Inc. as Amevive (tradename); darbepoetin alfa from Amgen Inc. as Aranesp (tradename); anastrozole from AstraZeneca Corp. as Arimidex (tradename); exemestane from Pfizer Inc. as Aromasin (tradename); bicalutamide from AstraZeneca Corp. as Casodex (tradename); leuprorelin from Takeda Pharmaceutical Co., Ltd. as Leuplin (tradename); flutamide from Schering-Plough Corp. as Eulexin (tradename); fulvestrant from AstraZeneca Corp. as Faslodex (tradename); pegaptanib octasodium from Gilead Sciences, Inc. as Macugen (tradename); denileukin diftitox from Ligand Pharmaceuticals Inc. as Ontak (tradename); aldesleukin from Chiron Corp. as Proleukin (tradename); thyrotropin alfa from Genzyme Corp. as Thyrogen (tradename); arsenic trioxide from Cell Therapeutics, Inc. as Trisenox (tradename); bortezomib from Millennium Pharmaceuticals, Inc. as Velcade (tradename); capecitabine from Hoffmann-La Roche, Ltd. as Xeloda (tradename); and goserelin from AstraZeneca Corp. as Zoladex (tradename). The term “antitumor agent” as used in the specification includes the above-described antitumor alkylating agent, antitumor antimetabolite, antitumor antibiotic, plant-derived antitumor agent, antitumor platinum coordination compound, antitumor camptothecin derivative, antitumor tyrosine kinase inhibitor, monoclonal antibody, interferon, biological response modifier, and other antitumor agents.

Other anti-tumor agents or anti-neoplastic agents can be used in combination with benzopyrone compounds. Such suitable anti-tumor agents or anti-neoplastic agents include, but are not limited to, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane, Actinomycin-D, Adriamycin, Aducil, Agrylin, Ala-Cort, Aldesleukin, Alemtuzumab, ALIMTA, Alitretinoin, Alkaban-AQ, Alkeran, All-transretinoic Acid, Alpha Interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arranon, Arsenic Trioxide, Asparaginase, ATRA, Avastin, Azacitidine, BCG, BCNU, Bendamustine, Bevacizumab, Bexarotene, BEXXAR, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine Wafer, Casodex, CC-5013, CCI-779, CCNU, CDDP, CeeNU, Cerubidine, Cetuximab, Chlorambucil, Cisplatin, Citrovorum Factor, Cladribine, Cortisone, Cosmegen, CPT-11, Cyclophosphamide, Cytadren, Cytarabine, Cytarabine Liposomal, Cytosar-U, Cytoxan, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome, Decadron, Decitabine, Delta-Cortef, Deltasone, Denileukin Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil, Doxorubicin, Doxorubicin Liposomal, Droxia™, DTIC, DTIC-Dome, Duralone, Efudex, Eligard, Ellence, Eloxatin, Elspar, Emcyt, Epirubicin, Epoetin Alfa, Erbitux, Erlotinib, Erwinia L-asparaginase, Estramustine, Ethyol, Etopophos, Etoposide, Etoposide Phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar & Gemzar Side Effects—Chemotherapy Drugs, Gleevec, Gliadel Wafer, GM-CSF, Goserelin, Granulocyte—Colony Stimulating Factor, Granulocyte Macrophage Colony Stimulating Factor, Halotestin, Herceptin, Hexadrol, Hexylen, Hexamethylmelamine, HMM, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin, Idarubicin, Ifex, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A (interferon alfa-2b), Iressa, Irinotecan, Isotretinoin, Ixabepilone, Ixempra, Kidrolase (t), Lanacort, Lapatinib, L-asparaginase, LCR, Lenalidomide, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine Hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mustargen, Mustine, Mutamycin, Myleran, Mylocel, Mylotarg, Navelbine, Nelarabine, Neosar, Neulasta, Neumega, Neupogen, Nexavar, Nilandron, Nilutamide, Nipent, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Paclitaxel Protein-bound, Pamidronate, Panitumumab, Panretin, Paraplatin, Pediapred, PEG Interferon, Pegaspargase, Pegfilgrastim, PEG-INTRON, PEG-L-asparaginase, PEMETREXED, Pentostatin, Phenylalanine Mustard, Platinol, Platinol-AQ, Prednisolone, Prednisone, Prelone, Procarbazine, PROCRIT, Proleukin, Prolifeprospan 20 with Carmustine Implant, Purinethol, Raloxifene, Revlimid, Rheumatrex, Rituxan, Rituximab, Roferon-A (Interferon Alfa-2a), Rubex, Rubidomycin hydrochloride, Sandostatin, Sandostatin LAR, Sargramostim, Solu-Cortef, Solu-Medrol, Sorafenib, SPRYCEL, STI-571, Streptozocin, SU11248, Sunitinib, Sutent, Tamoxifen, Tarceva, Targretin, Taxol, Taxotere, Temodar, Temozolomide, Temsirolimus, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Torisel, Tositumomab, Trastuzumab, Tretinoin, Trexall™, Trisenox, TSPA, TYKERB, VCR, Vectibix, Vectibix, Velban, Velcade, VePesid, Vesanoid, Viadur, Vidaza, Vinblastine, Vinblastine Sulfate, Vincasar Pfs, Vincristine, Vinorelbine, Vinorelbine tartrate, VLB, VM-26, Vorinostat, VP-16, Vumon, Xeloda, Zanosar, Zevalin, Zinecard, Zoladex, Zoledronic acid, Zolinza, Zometa.

Antimetabolites:

Antimetabolites are drugs that interfere with normal cellular metabolic processes. Since cancer cells are rapidly replicating, interference with cellular metabolism affects cancer cells to a greater extent than host cells. Gemcitabine (4-amino-1-[3,3-difluoro-4-hydroxy-5-(hydroxymethyl) tetrahydrofuran-2-yl]-1H-pyrimidin-2-one; marketed as GEMZAR® by Eli Lilly and Company) is a nucleoside analog, which interferes with cellular division by blocking DNA synthesis, thus resulting in cell death, apparently through an apoptotic mechanism. The dosage of gemcitabine may be adjusted to the particular patient. In adults, the dosage of gemcitabine, when used in combination with a platinum agent and a PARP inhibitor, will be in the range of about 100 mg/m² to about 5000 mg/m², in the range of about 100 mg/m² to about 2000 mg/m², in the range of about 750 to about 1500 mg/m², about 900 to about 1400 mg/m² or about 1250 mg/m². The dimensions mg/m² refer to the amount of gemcitabine in milligrams (mg) per unit surface area of the patient in square meters (m²). Gemcitabine may be administered by intravenous (IV) infusion, e.g. over a period of about 10 to about 300 minutes, about 15 to about 180 minutes, about 20 to about 60 minutes or about 10 minutes. The term “about” in this context indicates the normal usage of approximately; and in some embodiments indicates a tolerance of ±10% or ±5%.

Taxanes:

Taxanes are drugs that are derived from the twigs, needles and bark of Pacific yew tress, Taxus brevifolia. In particular paclitaxel may be derived from 10-deacetylbaccatin through known synthetic methods. Taxanes such as paclitaxel and its derivative docetaxel have demonstrated antitumor activity in a variety of tumor types. The taxanes interfere with normal function of microtubule growth by hyperstabilizing their structure, thereby destroying the cell's ability to use its cytoskeleton in a normal manner. Specifically, the taxanes bind to the β subunit of tubulin, which is the building block of microtubules. The resulting taxane/tubulin complex cannot disassemble, which results in aberrant cell function and eventual cell death. Paclitaxel induces programmed cell death (apoptosis) in cancer cells by binding to an apoptosis-inhibiting protein called Bcl-2 (B-cell leukemia 2), thereby preventing Bcl-2 from inhibiting apoptosis. Thus paclitaxel has proven to be an effective treatment for various cancers, as it down-regulates cell division by interrupting normal cytoskeletal rearrangement during cell division and it induces apoptosis via the anti-Bcl-2 mechanism.

The dosage of paclitaxel may vary depending upon the height, weight, physical condition, tumor size and progression state, etc. In some embodiments, the dosage of paclitaxel will be in the range of about 10 to about 2000 mg/m², about 10 to about 200 mg/m² or about 100 to about 175 mg/m². In some embodiments, the paclitaxel will be administered over a period of up to about 10 hours, up to about 8 hours or up to about 6 hours. The term “about” in this context indicates the normal usage of approximately; and in some embodiments indicates a tolerance of ±10% or ±5%.

Examples of taxanes include but are not limited to docetaxel, palitaxel, and Abraxane.

Platinum Complexes:

Platinum complexes are pharmaceutical compositions used to treat cancer, which contain at least one platinum center complexed with at least one organic group. Carboplatin ((SP-4-2)-Diammine[1,1-cyclobutanedicarboxylato(2-)-O,O′ platinum), like cisplatin and oxaliplatin, is a DNA alkylating agent. The dosage of carboplatin is determined by calculating the area under the blood plasma concentration curve (AUC) by methods known to those skilled in the cancer chemotherapy art, taking into account the patient's creatinine clearance rate. In some embodiments, the dosage of carboplatin for combination treatment along with a taxane (e.g. paclitaxel or docetaxel) and a PARP inhibitor (e.g. 4-iodo-3-nitrobenzamide) is calculated to provide an AUC of about 0.1-6 mg/ml·min, about 1-3 mg/ml·min, about 1.5 to about 2.5 mg/ml·min, about 1.75 to about 2.25 mg/ml·min or about 2 mg/ml·min. (AUC 2, for example, is shorthand for 2 mg/ml·min.) In some embodiments, a suitable carboplatin dose is about 10 to about 400 mg/m², e.g. about 360 mg/m². Platinum complexes, such as carboplatin, are normally administered intravenously (IV) over a period of about 10 to about 300 minutes, about 30 to about 180 minutes, about 45 to about 120 minutes or about 60 minutes. In this context, the term “about” has its normal meaning of approximately. In some embodiments, about means±10% or ±5%.

Topoisomerase Inhibitors

In some embodiments, the methods of the invention may comprise administering to a patient with uterine cancer or ovarian cancer an effective amount of a PARP inhibitor in combination with a topoisomerase inhibitor, for example, irinotecan and topotecan.

Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. Topoisomerases have become popular targets for cancer chemotherapy treatments. It is thought that topoisomerase inhibitors block the ligation step of the cell cycle, generating single and double stranded breaks that harm the integrity of the genome. Introduction of these breaks subsequently lead to apoptosis and cell death. Topoisomerase inhibitors are often divided according to which type of enzyme it inhibits. Topoisomerase I, the type of topoisomerase most often found in eukaryotes, is targeted by topotecan, irinotecan, lurtotecan and exatecan, each of which is commercially available from. Topotecan is available from GlaxoSmithKline under the trade name Hycamtim®. Irinotecan is available from Pfizer under the trade name Camptosar®. Lurtotecan may be obtained as a liposomal formulation from Gilead Sciences Inc. Topoisomerase inhibitors may be administered at an effective dose. In some embodiments an effective dose for treatment of a human will be in the range of about 0.01 to about 10 mg/m²/day. The treatment may be repeated on a daily, bi-weekly, semi-weekly, weekly, or monthly basis. In some embodiments, a treatment period may be followed by a rest period of from one day to several days, or from one to several weeks. In combination with a PARP-1 inhibitor, the PARP-1 inhibitor and the topoisomerase inhibitor may be dosed on the same day or may be dosed on separate days.

Compounds that target type II topoisomerase are split into two main classes: topoisomerase poisons, which target the topoisomerase-DNA complex, and topoisomerase inhibitors, which disrupt catalytic turnover. Topo II poisons include but are not limited to eukaryotic type II topoisomerase inhibitors (topo II): amsacrine, etoposide, etoposide phosphate, teniposide and doxorubicin. These drugs are anti-cancer therapies. Examples of topoisomerase inhibitors include ICRF-193. These inhibitors target the N-terminal ATPase domain of topo II and prevent topo II from turning over. The structure of this compound bound to the ATPase domain has been solved by Classen (Proceedings of the National Academy of Science, 2004) showing that the drug binds in a non-competitive manner and locks down the dimerization of the ATPase domain.

Anti-Angiogenic Agents

In some embodiments, the methods of the invention may comprise administering to a patient with uterine, endometrial, or ovarian cancer an effective amount of a PARP inhibitor in combination with an anti-angiogenic agent.

An angiogenesis inhibitor is a substance that inhibits angiogenesis (the growth of new blood vessels). Every solid tumor (in contrast to leukemia) needs to generate blood vessels to keep it alive once it reaches a certain size. Tumors can grow only if they form new blood vessels. Usually, blood vessels are not built elsewhere in an adult body unless tissue repair is actively in process. The angiostatic agent endostatin and related chemicals can suppress the building of blood vessels, preventing the cancer from growing indefinitely. In tests with patients, the tumor became inactive and stayed that way even after the endostatin treatment is finished. The treatment has very few side effects but appears to have very limited selectivity. Other angiostatic agents such as thalidomide and natural plant-based substances are being actively investigated.

Known inhibitors include the drug bevacizumab (Avastin), which binds vascular endothelial growth factor (VEGF), inhibiting its binding to the receptors that promote angiogenesis. Other anti-angiogenic agents include but are not limited to carboxyamidotriazole, TNF-470, CM101, IFN-alpha, IL-112, platelet factor-4, suramin, SU5416, thrombospondin, angiostatic steroids+heparin, cartilage-derived angiogenesis inhibitory factor, matrix metalloproteinase inhibitors, angiostatin, endostatin, 2-methoxyestradiol, tecogalan, thrombospondin, prolactin, α_{V}β₃ inhibitors and linomide.

### Her-2 Targeted Therapy

In some embodiments, the methods of the invention may comprise administering to a patient with HER2 positive uterine, endometrial, or ovarian cancer an effective amount of a PARP inhibitor in combination with Herceptin.

Her-2 overexpression has been found in ovarian carcinomas and HER2 overexpression and amplification is associated with advanced ovarian cancer (AOC)(Hellström et. al. Cancer Research 61, 2420-2423, Mar. 15, 2001). Overexpression of HER-2/neu in endometrial cancer is associated with advanced stage disease (Berchuck A, et. al. Am J Obstet. Gynecol. 1991 January; 164(1 Pt 1): 15-21). Herceptin may be used for the adjuvant treatment of HER2-overexpressing, uterine, endometrial, or ovarian cancers. Herceptin can be used several different ways: as part of a treatment regimen including doxorubicin, cyclophosphamide, and either paclitaxel or docetaxel; with docetaxel and carboplatin; or as a single agent following multi-modality anthracycline-based therapy. Herceptin in combination with paclitaxel is approved for the first-line treatment of HER2-overexpressing uterine, endometrial, or ovarian cancers. Herceptin as a single agent is approved for treatment of HER2-overexpressing uterine, endometrial, or ovarian cancer in patients who have received one or more chemotherapy regimens for metastatic disease.

Lapatinib or lapatinib ditosylate is an orally active chemotherapeutic drug treatment for solid tumours such as breast cancer. During development it was known as small molecule GW572016. Lapatinib may stop the growth of tumor cells by blocking some of the enzymes needed for cell growth. Drugs used in chemotherapy, such as topotecan, work in different ways to stop the growth of tumor cells, either by killing the cells or by preventing them from dividing. Giving lapatinib together with topotecan may have enhanced anti-tumor efficacy.

### Hormone Therapy

In some embodiments, the methods of the invention may comprise administering to a patient with uterine, endometrial, or ovarian cancer an effective amount of a PARP inhibitor in combination with hormone therapy.

Treatment for uterine cancer depends on the stage of the disease and the overall health of the patient. Removal of the tumor (surgical resection) is the primary treatment. Radiation therapy, hormone therapy, and/or chemotherapy may be used as adjuvant treatment (i.e., in addition to surgery) in patients with metastatic or recurrent disease.

Hormone therapy is used to treat metastatic or recurrent endometrial cancer. It also may be used to treat patients who are unable to undergo surgery or radiation. Prior to treatment, a hormone receptor test may be performed to determine if the endometrial tissue contains these proteins. Hormone therapy usually involves a synthetic type of progesterone in pill form. Estrogen can cause the growth of ovarian epithelial cancer cells. Thus, hormone therapy may be used to treat ovarian cancer.

Tamoxifen-Hormone Antagonist

Tamoxifen (marketed as Nolvadex) slows or stops the growth of cancer cells present in the body. Tamoxifen is a type of drug called a selective estrogen-receptor modulator (SERM). It functions as an anti-estrogen. As tamoxifen may have stabilized rapidly advancing recurrent ovarian cancer, its role in the primary treatment of ovarian cancer in combination with cytotoxic chemotherapy should be considered.

Steroidal and Non-Steroidal Aromatase Inhibitor

Aromatase inhibitors (AI) are a class of drugs used in the treatment of ovarian cancer in postmenopausal women that block the aromatase enzyme. Aromatase inhibitors lower the amount of estrogen in post-menopausal women who have hormone-receptor-positive ovarian cancer. With less estrogen in the body, the hormone receptors receive fewer growth signals, and cancer growth can be slowed down or stopped.

Aromatase inhibitor medications include Arimidex (chemical name: anastrozole), Aromasin (chemical name: exemestane), and Femara (chemical name: letrozole). Each is taken by pill once a day, for up to five years. But for women with advanced (metastatic) disease, the medicine is continued as long as it is working well.

AIs are categorized into two types: irreversible steroidal inhibitors such as exemestane that form a permanent bond with the aromatase enzyme complex; and non-steroidal inhibitors (such as anastrozole, letrozole) that inhibit the enzyme by reversible competition.

Fulvestrant, also known as ICI 182,780, and “Faslodex” is a drug treatment of hormone receptor-positive ovarian cancer in postmenopausal women with disease progression following anti-estrogen therapy. Estrogen can cause the growth of ovarian epithelial cancer cells. Fulvestrant is an estrogen receptor antagonist with no agonist effects, which works both by down-regulating and by degrading the estrogen receptor. It is administered as a once-monthly injection.

### Targeted Therapy

In some embodiments, the methods of the invention may comprise administering to a patient with uterine, endometrial, or ovarian cancer an effective amount of a PARP inhibitor in combination with an inhibitor targeting a growth factor receptor including but not limited to epidermal growth factor receptor (EGFR) and insulin-like growth factor I receptor (IGF1R).

EGFR is overexpressed in the cells of certain types of human carcinomas including but not limited to lung, breast, uterine, endometrial, and ovarian cancers. EGFR over-expression in ovarian cancer has been associated with poor prognosis. In addition, EGFR has been shown to be highly expressed in normal endometrium and overexpressed in endometrial cancer specimens, where it has been associated with a poor prognosis. Increased expression of EGFR may contribute to a drug resistant phenotype. The tyrosine kinase inhibitor ZD1839 (Iressa™) has been studied as a single agent in a phase II clinical trial (GOG 229C) of women with advanced endometrial cancer. Preliminary data analysis indicates that of 29 patients enrolled, 1 patient experienced a complete response and several others had stable disease at 6 months (Leslie, K.K.; et. al. International Journal of Gynecological Cancer, Volume 15, Number 2, 2005, pp. 409-411(3). Examples of EGFR inhibitors include but are not limited to cetuximab, which is a chimeric monoclonal antibody given by intravenous injection for treatment of cancers including but not limited to metastatic colorectal cancer and head and neck cancer. Panitumimab is another example of EGFR inhibitor. It is a humanized monoclonal antibody against EGFR. Panitumimab has been shown to be beneficial and better than supportive care when used alone in patients with advanced colon cancer and is approved by the FDA for this use.

Activation of the type I insulin-like growth factor receptor (IGFIR) promotes proliferation and inhibits apoptosis in a variety of cell types. One example of an IGF1R inhibitor is CP-751871. CP-751871 is a human monoclonal antibody that selectively binds to IGF1R, preventing IGF1 from binding to the receptor and subsequent receptor autophosphorylation. Inhibition of IGF1R autophosphorylation may result in a reduction in receptor expression on tumor cells that express IGF1R, a reduction in the anti-apoptotic effect of IGF, and inhibition of tumor growth. IGF1R is a receptor tyrosine kinase expressed on most tumor cells and is involved in mitogenesis, angiogenesis, and tumor cell survival.

### PI3K/mTOR Pathway

Phosphatidylinositol-3-kinase (PI3K) pathway deregulation is a common event in human cancer, either through inactivation of the tumor suppressor phosphatase and tensin homologue deleted from chromosome 10 or activating mutations of p110-α. These hotspot mutations result in oncogenic activity of the enzyme and contribute to therapeutic resistance to the anti-HER2 antibody trastuzumab. Akt and mTOR phosphorylation is also frequently detected in ovarian and endometrial cancer. The PI3K pathway is, therefore, an attractive target for cancer therapy. NVP-BEZ235, a dual inhibitor of the PI3K and the downstream mammalian target of rapamycin (mTOR) has been shown to have antiproliferative and antitumoral activity in cancer cells with both wild-type and mutated p110-α (Violeta Serra, et. al. Cancer Research 68, 8022-8030, Oct. 1, 2008).

### Hsp90 Inhibitors

These drugs target heat shock protein 90 (hsp90). Hsp90 is one of a class of chaperone proteins, whose normal job is to help other proteins acquire and maintain the shape required for those proteins to do their jobs. Chaperone proteins work by being in physical contact with other proteins. Hsp90 can also enable cancer cells to survive and even thrive despite genetic defects which would normally cause such cells to die. Thus, blocking the function of HSP90 and related chaperone proteins may cause cancer cells to die, especially if blocking chaperone function is combined with other strategies to block cancer cell survival.

### Tubulin Inhibitors

Tubulins are the proteins that form microtubules, which are key components of the cellular cytoskeleton (structural network). Microtubules are necessary for cell division (mitosis), cell structure, transport, signaling and motility. Given their primary role in mitosis, microtubules have been an important target for anticancer drugs—often referred to as antimitotic drugs, tubulin inhibitors and microtubule targeting agents. These compounds bind to tubulin in microtubules and prevent cancer cell proliferation by interfering with the microtubule formation required for cell division. This interference blocks the cell cycle sequence, leading to apoptosis.

### Apoptosis Inhibitors

The inhibitors of apoptosis (IAP) are a family of functionally- and structurally-related proteins, originally characterized in Baculovirus, which serve as endogenous inhibitors of apoptosis. The human IAP family consists of at least 6 members, and IAP homologs have been identified in numerous organisms. 10058-F4 is a c-Myc inhibitor that induces cell-cycle arrest and apoptosis. It is a cell-permeable thiazolidinone that specifically inhibits the c-Myc-Max interaction and prevents transactivation of c-Myc target gene expression. 10058-F4 inhibits tumor cell growth in a c-Myc-dependent manner both in vitro and in vivo. BI-6C9 is a tBid inhibitor and antiapoptotic. GNF-2 belongs to a new class of Bcr-abl inhibitors. GNF-2 appears to bind to the myristoyl binding pocket, an allosteric site distant from the active site, stabilizing the inactive form of the kinase. It inhibits Bcr-abl phosphorylation with an IC₅₀ of 267 nM, but does not inhibit a panel of 63 other kinases, including native c-Abl, and shows complete lack of toxicity towards cells not expressing Bcr-Abl. GNF-2 shows great potential for a new class of inhibitor to study Bcr-abl activity and to treat resistant Chronic myelogenous leukemia (CML), which is caused the Bcr-Abl oncoprotein. Pifithrin-α is a reversible inhibitor of p53-mediated apoptosis and p53-dependent gene transcription such as cyclin G, p21/waf1, and mdm2 expression. Pifithrin-α enhances cell survival after genotoxic stress such as UV irradiation and treatment with cytotoxic compounds including doxorubicin, etopoxide, paclitaxel, and cytosine-β-D-arabinofuranoside. Pifithrin-α protects mice from lethal whole body γ-irradiation without an increase in cancer incidence.

PARP Inhibitors:

In some embodiments, the present invention provides a method of treating uterine cancer or ovarian cancer by administering to a subject in need thereof at least one PARP inhibitor. In other embodiments, the present invention provides a method of treating uterine cancer or ovarian cancer by administering to a subject in need thereof at least one PARP inhibitor in combination with at least one anti-tumor agent described herein.

Not intending to be limited to any particular mechanism of action, the compounds described herein are believed to have anti-cancer properties due to the modulation of activity of a poly (ADP-ribose) polymerase (PARP). This mechanism of action is related to the ability of PARP inhibitors to bind PARP and decrease its activity. PARP catalyzes the conversion of β-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J, et al. 1997, Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology—directed DSB repair (Bryant H E, et al. (2005) Nature 434:913-917; Farmer H, et al. (2005) Nature 434:917-921).

BRCA1 and BRCA2 act as an integral component of the homologous recombination machinery (HR) (Narod S A, Foulkes W D (2004) Nat Rev Cancer 4:665-676; Gudmundsdottir K, Ashworth A (2006) Oncogene 25:5864-5874).

Cells defective in BRCA1 or BRCA2 have a defect in the repair of double-strand breaks (DSB) by the mechanism of homologous recombination (HR) by gene conversion (Farmer H, et al. (2005) Nature 434:917-921; Narod S A, Foulkes W D (2004) Nat Rev Cancer 4:665-676; Gudmundsdottir K, Ashworth A (2006) Oncogene 25:5864-5874; Helleday T, et al. (2008) Nat Rev Cancer 8:193-204). Deficiency in either of the breast cancer susceptibility proteins BRCA1 or BRCA2 induces profound cellular sensitivity to the inhibition of poly(ADP-ribose) polymerase (PARP) activity, resulting in cell cycle arrest and apoptosis. It has been reported that the critical role of BRCA1 and BRCA2 in the repair of double-strand breaks by homologous recombination (HR) is the underlying reason for this sensitivity, and the deficiency of RAD51, RAD54, DSS1, RPA1, NBS1, ATR, ATM, CHK1, CHK2, FANCD2, FANCA, or FANCC induces such sensitivity (McCabe N. et. al. Deficiency in the repair of DNA damage by homologous recombination and sensitivity to poly(ADP-ribose) polymerase inhibition, Cancer research 2006, vol. 66, 8109-8115). It has been proposed that PARP1 inhibition can be a specific therapy for cancers with defects in BRCA112 or other HR pathway components (Helleday T, et al. (2008) Nat Rev Cancer 8:193-204). Uterine tumors and ovarian tumors frequently harbor defects in DNA double-strand break repair through homologous recombination (HR), such as BRCA1 dysfunction (Rottenberg S, et. al. Proc Natl Acad Sci USA. 2008 Nov. 4; 105(44):17079-84).

Inhibiting the activity of a PARP molecule includes reducing the activity of these molecules. The term “inhibits” and its grammatical conjugations, such as “inhibitory,” is not intended to require complete reduction in PARP activity. In some embodiments, such reduction is at least about 50%, at least about 75%, at least about 90%, or at least about 95% of the activity of the molecule in the absence of the inhibitory effect, e.g., in the absence of an inhibitor, such as a nitrobenzamide compound of the invention. In some embodiments, inhibition refers to an observable or measurable reduction in activity. In treatment some scenarios, the inhibition is sufficient to produce a therapeutic and/or prophylactic benefit in the condition being treated. The phrase “does not inhibit” and its grammatical conjugations does not require a complete lack of effect on the activity. For example, it refers to situations where there is less than about 20%, less than about 10%, and preferably less than about 5% of reduction in PARP activity in the presence of an inhibitor such as a nitrobenzamide compound of the invention.

Poly (ADP-ribose) polymerase (PARP) is an essential enzyme in DNA repair, thus playing a potential role in chemotherapy resistance. Targeting PARP potentially is thought to interrupt DNA repair, thereby enhancing taxane mediated-, antimetabolite mediated-, topoisomerase inhibitor-mediated, and growth factor receptor inhibitor, e.g. IGF1R inhibitor-mediated, and/or platinum complex mediated-DNA replication and/or repair in cancer cells. PARP inhibitors may also be highly active against ovarian cancer, uterine cancer, and endometrial cancer with impaired function of BRCA 1 and BRCA2 or those patients with other DNA repair pathway defects.

4-Iodo-3-nitrobenzamide (BA) is a small molecule that acts on tumor cells without exerting toxic effects in normal cells. BA is believed to achieve its anti-neoplastic effect by inhibition of PARP. BA is very lipophilic and distributes rapidly and widely into tissues, including the brain and cerebrospinal fluid (CSF). It is active against a broad range of cancer cells in vitro, including against drug resistant cell lines. The person skilled in the art will recognize that BA may be administered in any pharmaceutically acceptable form, e.g. as a pharmaceutically acceptable salt, solvate, or complex. Additionally, as BA is capable of tautomerizing in solution, the tautomeric form of BA is intended to be embraced by the term BA (or the equivalent 4-iodo-3-nitrobenzamide), along with the salts, solvates or complexes. In some embodiments, BA may be administered in combination with a cyclodextrin, such as hydroxypropylbetacyclodextrin. However, one skilled in the art will recognize that other active and inactive agents may be combined with BA; and recitation of BA will, unless otherwise stated, include all pharmaceutically acceptable forms thereof.

Basal-like endometrial cancers have a high propensity to metastasize to the brain; and BA is known to cross the blood-brain barrier. While not wishing to be bound by any particular theory, it is believed that BA achieves its anti-neoplastic effect by inhibiting the function of PARP. In some embodiments, BA can be used in the treatment of metastatic ovarian cancer. In some embodiments, BA can be used in the treatment of metastatic uterine cancer. In some embodiments, BA can be used in the treatment of metastatic endometrial cancer. In other embodiments, BA can be used in the treatment of uterine, endometrial, or ovarian tumors in combination with an anti-tumor agent. In some embodiments, the anti-tumor agent is an antimetabolite such as gemcitabine. In some embodiments, the anti-tumor agent is a platinum complex such as carboplatin. In some embodiments, BA can be used in the treatment of uterine, endometrial, or ovarian tumors in combination with a taxane such as paclitaxel. In other embodiments, BA can be used in the treatment of uterine, endometrial, or ovarian tumors in combination with an anti-angiogenic agent. In still other embodiments, BA can be used in the treatment of uterine, endometrial, or ovarian tumors in combination with a topoisomerase inhibitor such as irinotecan. In other embodiments, BA can be used in the treatment of uterine, endometrial, or ovarian tumors in combination with hormone therapy. In still other embodiments, BA can be used in the treatment of uterine, endometrial, or ovarian tumors in combination with a growth factor receptor inhibitor including but not limited to EGFR or IGF1R inhibitor. In some embodiments, the uterine, endometrial, or ovarian cancer is a metastatic cancer.

The dosage of PARP inhibitor may vary depending upon the patient age, height, weight, overall health, etc. In some embodiments, the dosage of BA is in the range of about 1 mg/kg to about 100 mg/kg, about 2 mg/kg to about 50 mg/kg, about 2 mg/kg, about 4 mg/kg, about 6 mg/kg, about 8 mg/kg, about 10 mg/kg, about 12 mg/kg, about 15 mg/kg, about 20 mg/kg, about 25 mg/kg, about 30 mg/kg, about 35 mg/kg, about 40 mg/kg, about 50 mg/kg, about 60 mg/kg, about 75 mg/kg, about 90 mg/kg, about 1 to about 25 mg/kg, about 2 to about 70 mg/kg, about 4 to about 100 mg, about 4 to about 25 mg/kg, about 4 to about 20 mg/kg, about 50 to about 100 mg/kg or about 25 to about 75 mg/kg. BA may be administered intravenously, e.g. by IV infusion over about 10 to about 300 minutes, about 30 to about 180 minutes, about 45 to about 120 minutes or about 60 minutes (i.e. about 1 hour). In some embodiments, BA may alternatively be administered orally. In this context, the term “about” has its normal meaning of approximately. In some embodiments, about means±10% or ±5%.

The synthesis of BA (4-iodo-3-nitrobenzamide) is described in U.S. Pat. No. 5,464,871, which is incorporated herein by reference in its entirety. BA may be prepared in concentrations of 10 mg/mL and may be packaged in a convenient form, e.g. in 10 mL vials.

BA Metabolites:

As used herein “BA” means 4-iodo-3-nitrobenzamide; “BNO” means 4-iodo-3-nitrosobenzamide; “BNHOH” means 4-iodo-3-hydroxyaminobenzamide.

Precursor compounds useful in the present invention are of Formula (Ia)

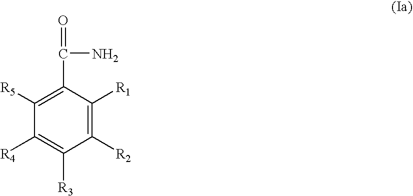

wherein R₁, R₂, R₃, R₄, and R₅ are, independently selected from the group consisting of hydrogen, hydroxy, amino, nitro, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or pro-drugs thereof. R₁, R₂, R₃, R₄, and R₅ can also be a halide such as chloro, fluoro, or bromo substituents.

A preferred precursor compound of formula Ia is:

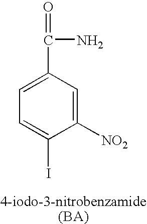

Some metabolites useful in the present invention are of the Formula (IIa):

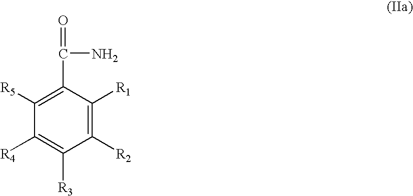

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

The following compositions are preferred metabolite compounds, each represented by a chemical formula:

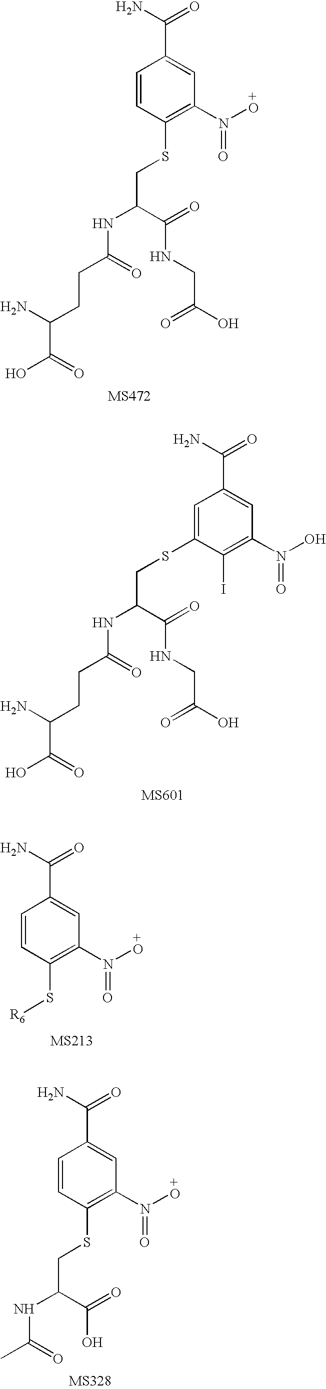

R₆ is selected from a group consisting of hydrogen, alkyl(C₁-C₈), alkoxy (C₁-C₈), isoquinolinones, indoles, thiazole, oxazole, oxadiazole, thiphene, or phenyl.

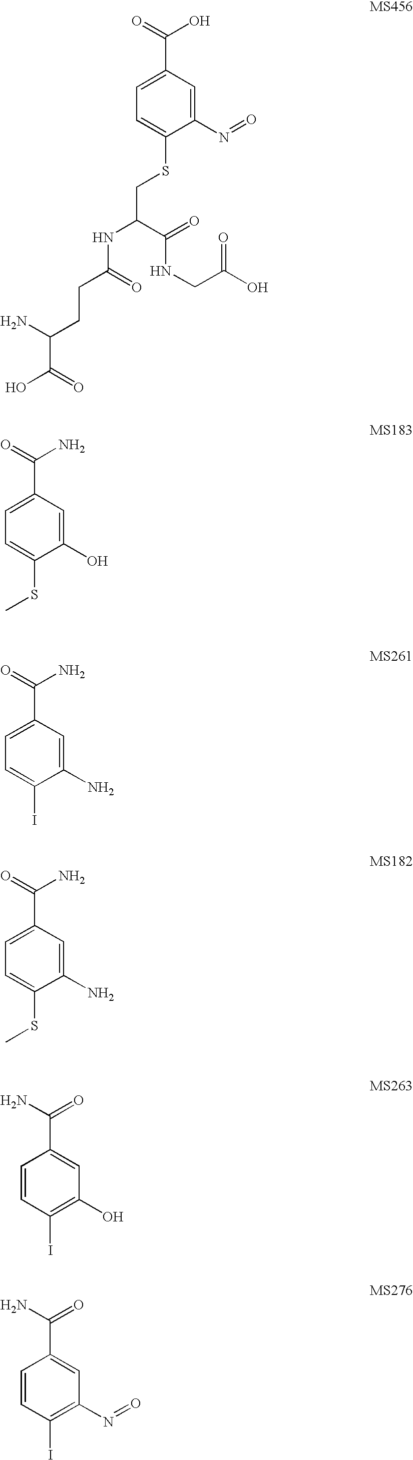

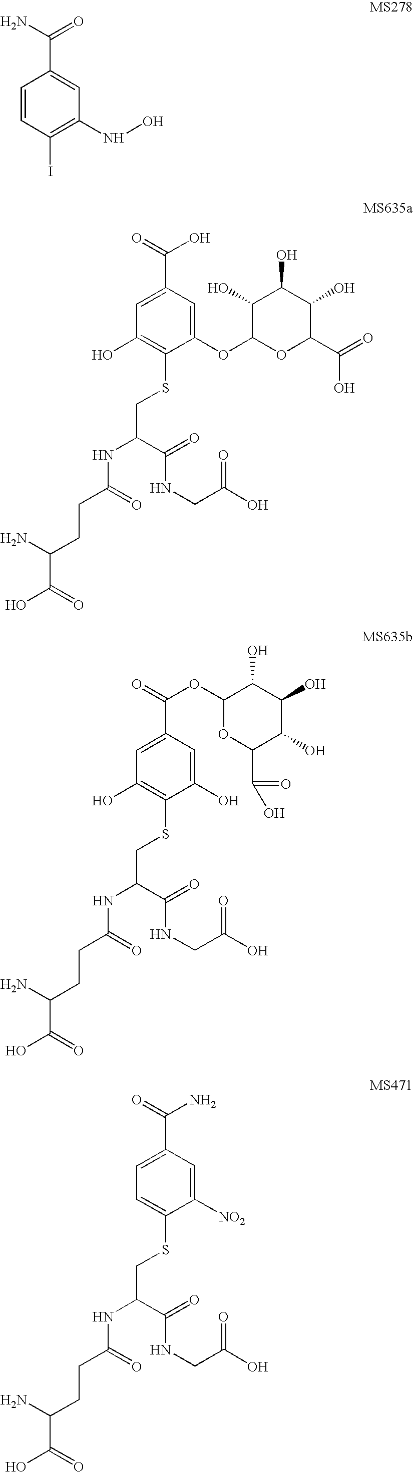

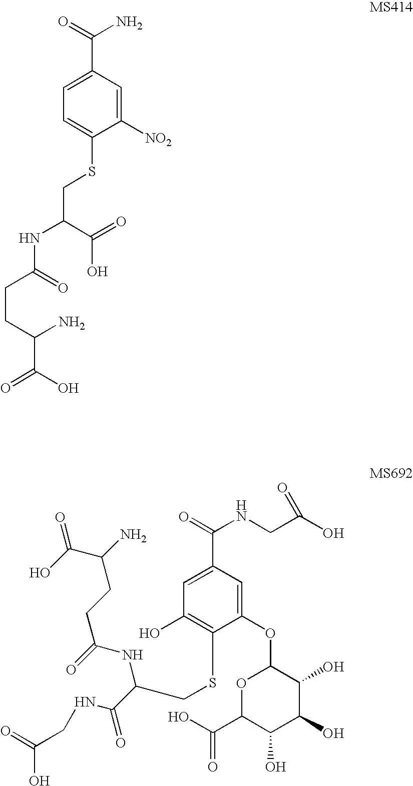

While not being limited to any one particular mechanism, the following provides an example for MS292 metabolism via a nitroreductase or glutathione conjugation mechanism:

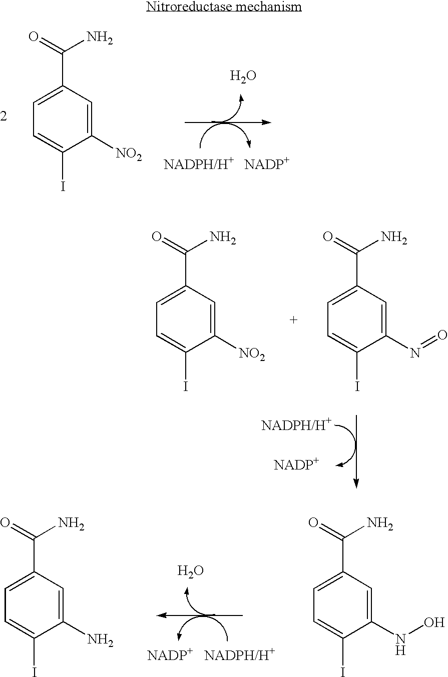

BA glutathione conjugation and metabolism:

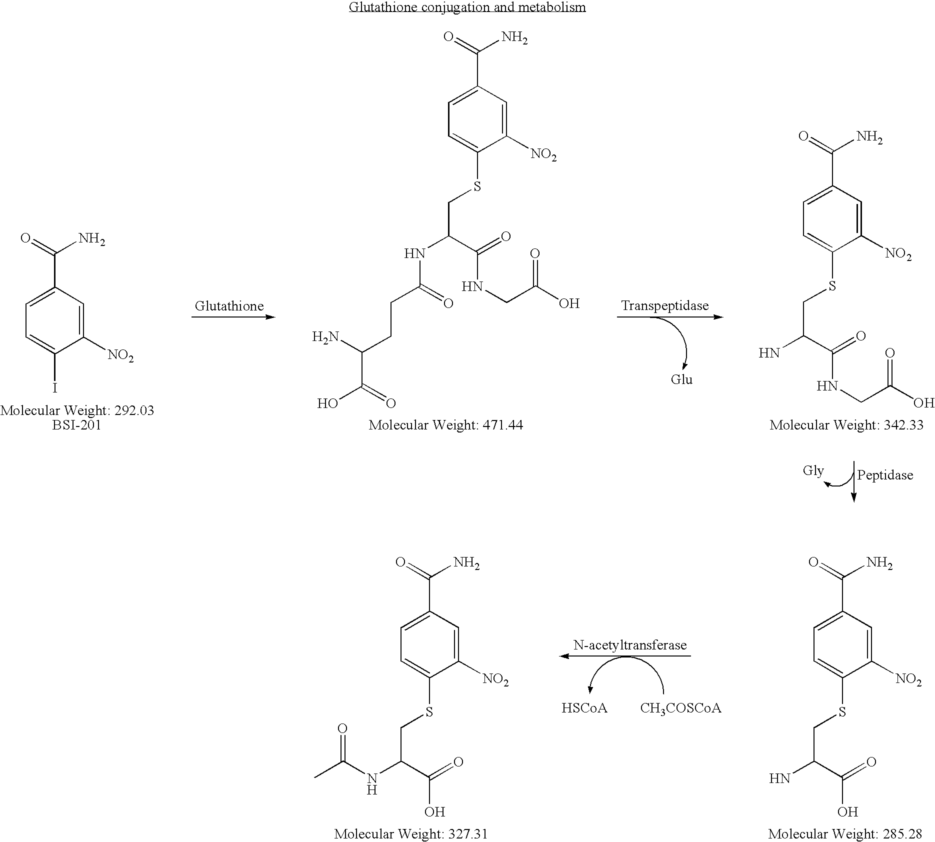

The present invention provides for the use of the aforesaid nitrobenzamide metabolite compounds for the treatment of ovarian cancer with a genetic defect in a BRCA gene, or a uterine cancer that is recurrent, advanced or persistent.

It has been reported that nitrobenzamide metabolite compounds have selective cytotoxicity upon malignant cancer cells but not upon non-malignant cancer cells. See Rice et at., Proc. Natl. Acad. Sci. USA 89:7703-7707 (1992), incorporated herein in it entirety. In one embodiment, the nitrobenzamide metabolite compounds utilized in the methods of the present invention may exhibit more selective toxicity towards tumor cells than non-tumor cells. The metabolites according to the invention may thus be administered to a patient in need of such treatment in conjunction with chemotherapy with at least one taxane (e.g. paclitaxel or docetaxel) in addition to the at least one platinum complex (e.g. carboplatin, cisplatin, etc.) The dosage range for such metabolites may be in the range of about 0.0004 to about 0.5 mmol/kg (millimoles of metabolite per kilogram of patient body weight), which dosage corresponds, on a molar basis, to a range of about 0.1 to about 100 mg/kg of BA. Other effective ranges of dosages for metabolites are 0.0024-0.5 mmol/kg and 0.0048-0.25 mmol/kg. Such doses may be administered on a daily, every-other-daily, twice-weekly, weekly, bi-weekly, monthly or other suitable schedule. Essentially the same modes of administration may be employed for the metabolites as for BA—e.g. oral, i.v., i.p., etc.

### Combination Therapy

In certain embodiments of the present invention, the methods of the invention further comprise treating uterine cancer, endometrial cancer, or ovarian cancer by administering to a subject a PARP inhibitor with or without at least one anti-tumor agent in combination with another anti-cancer therapy including but not limited to surgery, radiation therapy (e.g. X ray), gene therapy, DNA therapy, adjuvant therapy, neoadjuvant therapy, viral therapy, immunotherapy, RNA therapy, or nanotherapy.

Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, by a significant period of time. The conjugate and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

### Radiation Therapy

Radiation therapy (or radiotherapy) is the medical use of ionizing radiation as part of cancer treatment to control malignant cells. Radiotherapy may be used for curative or adjuvant cancer treatment. It is used as palliative treatment (where cure is not possible and the aim is for local disease control or symptomatic relief) or as therapeutic treatment (where the therapy has survival benefit and it can be curative). Radiotherapy is used for the treatment of malignant tumors and may be used as the primary therapy. It is also common to combine radiotherapy with surgery, chemotherapy, hormone therapy or some mixture of the three. Most common cancer types can be treated with radiotherapy in some way. The precise treatment intent (curative, adjuvant, neoadjuvant, therapeutic, or palliative) will depend on the tumour type, location, and stage, as well as the general health of the patient.

Radiation therapy is commonly applied to the cancerous tumor. The radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion.

Radiation therapy works by damaging the DNA of cells. The damage is caused by a photon, electron, proton, neutron, or ion beam directly or indirectly ionizing the atoms which make up the DNA chain. Indirect ionization happens as a result of the ionization of water, forming free radicals, notably hydroxyl radicals, which then damage the DNA. In the most common forms of radiation therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly. Proton radiotherapy works by sending protons with varying kinetic energy to precisely stop at the tumor.

Gamma rays are also used to treat some types of cancer including uterine, endometrial, and ovarian cancers. In the procedure called gamma-knife surgery, multiple concentrated beams of gamma rays are directed on the growth in order to kill the cancerous cells. The beams are aimed from different angles to focus the radiation on the growth while minimizing damage to the surrounding tissues.

### Gene Therapy Agents

Gene therapy agents insert copies of genes into a specific set of a patient's cells, and can target both cancer and non-cancer cells. The goal of gene therapy can be to replace altered genes with functional genes, to stimulate a patient's immune response to cancer, to make cancer cells more sensitive to chemotherapy, to place “suicide” genes into cancer cells, or to inhibit angiogenesis. Genes may be delivered to target cells using viruses, liposomes, or other carriers or vectors. This may be done by injecting the gene-carrier composition into the patient directly, or ex vivo, with infected cells being introduced back into a patient. Such compositions are suitable for use in the present invention.

### Adjuvant Therapy

Adjuvant therapy is a treatment given after the primary treatment to increase the chances of a cure. Adjuvant therapy may include chemotherapy, radiation therapy, hormone therapy, or biological therapy.

Adjuvant chemotherapy is effective for patients with advanced uterine cancer or ovarian cancer. The combination of doxorubicin and cisplatin achieves overall response rates ranging from 34 to 60%, and the addition of paclitaxel seems to improve the outcome of patients with advanced disease, but it induces a significantly higher toxicity. A Gynecologic Oncology Study Group phase-III study is currently exploring the triplet paclitaxel+doxorubicin+cisplatin plus G-CSF vs. the less toxic combination of paclitaxel+carboplatin. Ongoing and planned phase-III trials are evaluating newer combination chemotherapy regimens, a combination of irradiation and chemotherapy and the implementation of targeted therapies with the goal of improving the tumor control rate and quality of life.

Adjuvant radiation therapy (RT)—Adjuvant radiation therapy significantly reduces the risk that the uterine cancer will recur locally (ie, in the pelvis or vagina). In general, there are two ways of delivering RT: it may be given as vaginal brachytherapy or as external beam RT (EBRT). In vaginal brachytherapy, brachytherapy delivers RT directly to the vaginal tissues from a source that is temporarily placed inside the body. This allows high doses of radiation to be delivered to the area where cancer cells are most likely to be found. With external beam radiation therapy (EBRT), the source of the radiation is outside the body.

Various therapies including but not limited to hormone therapy, e.g. tamoxifen, or gonadotropin-releasing hormone (GnRH) analogues, and radioactive monoclonal antibody therapy have been used to treat ovarian cancer.

### Neoadjuvant Therapy

Neoadjuvant therapy refers to a treatment given before the primary treatment. Examples of neoadjuvant therapy include chemotherapy, radiation therapy, and hormone therapy. Neoadjuvant chemotherapy in gynecological cancers is an approach that is shown to have positive effects on survival. It increases the rate of resectability in ovarian and cervical cancers and thus contributes to survival (Ayhan A. et. al. European journal of gynaecological oncology. 2006, vol. 27).

### Oncolytic Viral Therapy

Viral therapy for cancer utilizes a type of viruses called oncolytic viruses. An oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site.

There are two main approaches for generating tumor selectivity: transductional and non-transductional targeting. Transductional targeting involves modifying the specificity of viral coat protein, thus increasing entry into target cells while reducing entry to non-target cells. Non-transductional targeting involves altering the genome of the virus so it can only replicate in cancer cells. This can be done by either transcription targeting, where genes essential for viral replication are placed under the control of a tumor-specific promoter, or by attenuation, which involves introducing deletions into the viral genome that eliminate functions that are dispensable in cancer cells, but not in normal cells. There are also other, slightly more obscure methods.

Chen et al (2001) used CV706, a prostate-specific adenovirus, in conjunction with radiotherapy on prostate cancer in mice. The combined treatment results in a synergistic increase in cell death, as well as a significant increase in viral burst size (the number of virus particles released from each cell lysis).

ONYX-015 has undergone trials in conjunction with chemotherapy. The combined treatment gives a greater response than either treatment alone, but the results have not been entirely conclusive. ONYX-015 has shown promise in conjunction with radiotherapy.

Viral agents administered intravenously can be particularly effective against metastatic cancers, which are especially difficult to treat conventionally. However, bloodborne viruses can be deactivated by antibodies and cleared from the blood stream quickly e.g. by Kupffer cells (extremely active phagocytic cells in the liver, which are responsible for adenovirus clearance). Avoidance of the immune system until the tumour is destroyed could be the biggest obstacle to the success of oncolytic virus therapy. To date, no technique used to evade the immune system is entirely satisfactory. It is in conjunction with conventional cancer therapies that oncolytic viruses show the most promise, since combined therapies operate synergistically with no apparent negative effects.

The specificity and flexibility of oncolytic viruses means they have the potential to treat a wide range of cancers including uterine cancer, endometrial cancer, and ovarian cancer with minimal side effects. Oncolytic viruses have the potential to solve the problem of selectively killing cancer cells.

### Nanotherapy

Nanometer-sized particles have novel optical, electronic, and structural properties that are not available from either individual molecules or bulk solids. When linked with tumor-targeting moieties, such as tumor-specific ligands or monoclonal antibodies, these nanoparticles can be used to target cancer-specific receptors, tumor antigens (biomarkers), and tumor vasculatures with high affinity and precision. The formuation and manufacturing process for cancer nanotherapy is disclosed in U.S. Pat. No. 7,179,484, and article M. N. Khalid, P. Simard, D. Hoarau, A. Dragomir, J. Leroux, Long Circulating Poly(Ethylene Glycol)Decorated Lipid Nanocapsules Deliver Docetaxel to Solid Tumors, Pharmaceutical Research, 23(4), 2006, all of which are herein incorporated by reference in their entireties.

### RNA Therapy

RNA including but not limited to siRNA, shRNA, microRNA may be used to modulate gene expression and treat cancers. Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

Certain RNA inhibiting agents may be utilized to inhibit the expression or translation of messenger RNA (“mRNA”) that is associated with a cancer phenotype. Examples of such agents suitable for use herein include, but are not limited to, short interfering RNA (“siRNA”), ribozymes, and antisense oligonucleotides. Specific examples of RNA inhibiting agents suitable for use herein include, but are not limited to, Cand5, Sirna-027, fomivirsen, and angiozyme.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives.

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Chemopreventative Agents

Certain pharmaceutical agents can be used to prevent initial occurrences of cancer, or to prevent recurrence or metastasis. Administration with such chemopreventative agents in combination with eflomithine-NSAID conjugates of the invention can act to both treat and prevent the recurrence of cancer. Examples of chemopreventative agents suitable for use herein include, but are not limited to, tamoxifen, raloxifene, tibolone, bisphosphonate, ibandronate, estrogen receptor modulators, aromatase inhibitors (letrozole, anastrozole), luteinizing hormone-releasing hormone agonists, goserelin, vitamin A, retinal, retinoic acid, fenretinide, 9-cis-retinoid acid, 13-cis-retinoid acid, all-trans-retinoic acid, isotretinoin, tretinoid, vitamin B6, vitamin B12, vitamin C, vitamin D, vitamin E, cyclooxygenase inhibitors, non-steroidal anti-inflammatory drugs (NSAIDs), aspirin, ibuprofen, celecoxib, polyphenols, polyphenol E, green tea extract, folic acid, glucaric acid, interferon-alpha, anethole dithiolethione, zinc, pyridoxine, finasteride, doxazosin, selenium, indole-3-carbinal, alpha-difluoromethylomithine, carotenoids, beta-carotene, lycopene, antioxidants, coenzyme Q10, flavonoids, quercetin, curcumin, catechins, epigallocatechin gallate, N-acetylcysteine, indole-3-carbinol, inositol hexaphosphate, isoflavones, glucanic acid, rosemary, soy, saw palmetto, and calcium. An additional example of chemopreventative agents suitable for use in the present invention is cancer vaccines. These can be created through immunizing a patient with all or part of a cancer cell type that is targeted by the vaccination process.

Clinical Efficacy:

Clinical efficacy may be measured by any method known in the art. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD≧6 months. The CBR for combination therapy with paclitaxel and carboplatin is 45%. Thus, the CBR for triple combination therapy with a taxane, platinum complex and PARP inhibitor (e.g. paclitaxel, carboplatin and BA; CBR_{GCB}) may be compared to that of the double combination therapy with paclitaxel and carboplatin (CBR_{GC}). In some embodiments, CBR_{GCB} is at least about 60%. In some embodiments, the CBR is at least about 30%, at least about 40%, or at least about 50%.

In some embodiments disclosed herein, the methods include pre-determining that a cancer is treatable by PARP modulators. Some such methods comprise identifying a level of PARP in a uterine, endometrial, or ovarian cancer sample of a patient, determining whether the level of PARP expression in the sample is greater than a pre-determined value, and, if the PARP expression is greater than said predetermined value, treating the patient with a combination of an anti-tumor agent described herein and a PARP inhibitor such as BA. In other embodiments, the methods comprise identifying a level of PARP in a uterine, endometrial, or ovarian cancer sample of a patient, determining whether the level of PARP expression in the sample is greater than a pre-determined value, and, if the PARP expression is greater than said predetermined value, treating the patient with a PARP inhibitor, such as BA.

Uterine tumors in women who inherit faults in either the BRCA1 or BRCA2 genes occur because the tumor cells have lost a specific mechanism that repair damaged DNA. BRCA1 and BRCA2 are important for DNA double-strand break repair by homologous recombination, and mutations in these genes predispose to uterine and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis (Jones C, Plummer E R. PARP inhibitors and cancer therapy—early results and potential applications. Br J Radiol. 2008 October; 81 Spec No 1:S2-5; Drew Y, Calvert H. The potential of PARP inhibitors in genetic breast and ovarian cancers. Ann N Y Acad. Sci. 2008 September; 1138:136-45; Farmer H, et. al. Targeting the DNA repair defect in BRCA mutant cells as a therapeutic strategy. Nature. 2005 Apr. 14; 434(7035):917-21).

Patients deficient in BRCA genes may have up-regulated levels of PARP. PARP up-regulation may be an indicator of other defective DNA-repair pathways and unrecognized BRCA-like genetic defects. Assessment of PARP gene expression and impaired DNA repair especially defective homologous recombination DNA repair can be used as an indicator of tumor sensitivity to PARP inhibitor. Hence, in some embodiments, treatment of uterine cancer can be enhanced not only by determining the HR and/or HER2 status of the cancer, but also by identifying early onset of cancer in BRCA and homologous recombination DNA repair deficient patients by measuring the level of PARP. The BRCA and homologous recombination DNA repair deficient patients treatable by PARP inhibitors can be identified if PARP is up-regulated. Further, such homologous recombination DNA repair deficient patients can be treated with PARP inhibitors.

In some embodiments, a sample is collected from a patient having a uterine lesion suspected of being cancerous. While such sample may be any available biological tissue, in most cases the sample will be a portion of the suspected uterine lesion, whether obtained by laparoscopy or open surgery (e.g. hysterectomy). PARP expression may then be analyzed and, if the PARP expression is above a predetermined level (e.g. is up-regulated vis-á-vis normal tissue) the patient may be treated with a PARP inhibitor in combination with a taxane and a platinum agent. It is thus to be understood that, while embodiments described herein are directed to treatment of endometrial cancer, recurrent, advanced, or persistent uterine cancer, and ovarian cancer in association with a BRCA-defect, in some embodiments, the uterine or ovarian cancer need not have these characteristics so long as the threshold PARP up-regulation is satisfied.

In some embodiments, tumors that are homologous recombination deficient are identified by evaluating levels of PARP expression. If up-regulation of PARP is observed, such tumors can be treated with PARP inhibitors. Another embodiment is a method for treating a homologous recombination deficient cancer comprising evaluating level of PARP expression and, if overexpression is observed, the cancer is treated with a PARP inhibitor.

### Sample Collection, Preparation and Separation

Biological samples may be collected from a variety of sources from a patient including a body fluid sample, or a tissue sample. Samples collected can be human normal and tumor samples, nipple aspirants. The samples can be collected from individuals repeatedly over a longitudinal period of time (e.g., about once a day, once a week, once a month, biannually or annually). Obtaining numerous samples from an individual over a period of time can be used to verify results from earlier detections and/or to identify an alteration in biological pattern as a result of, for example, disease progression, drug treatment, etc.

Sample preparation and separation can involve any of the procedures, depending on the type of sample collected and/or analysis of PARP. Such procedures include, by way of example only, concentration, dilution, adjustment of pH, removal of high abundance polypeptides (e.g., albumin, gamma globulin, and transferin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g. aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC) etc.

Identifying Level of PARP

The poly (ADP-ribose) polymerase (PARP) is also known as poly (ADP-ribose) synthase and poly ADP-ribosyltransferase. PARP catalyzes the formation of poly (ADP-ribose) polymers which can attach to cellular proteins (as well as to itself) and thereby modify the activities of those proteins. The enzyme plays a role in enhancing DNA repair, but it also plays a role in regulation of transcription, cell proliferation, and chromatin remodeling (for review see: D. D'amours et al. “Poly (ADP-ribosylation reactions in the regulation of nuclear functions,” Biochem. J. 342: 249-268 (1999)).

PARP-1 comprises an N-terminal DNA binding domain, an automodification domain and a C-terminal catalytic domain and various cellular proteins interact with PARP-1. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP-1 itself interact with PARP-1 in this domain. The automodification domain contains a BRCT motif, one of the protein-protein interaction modules. This motif is originally found in the C-terminus of BRCA1 (uterine cancer susceptibility protein 1) and is present in various proteins related to DNA repair, recombination and cell-cycle checkpoint control. POU-homeodomain-containing octamer transcription factor-1 (Oct-1), Yin Yang (YY)1 and ubiquitin-conjugating enzyme 9 (ubc9) could interact with this BRCT motif in PARP-1.

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and may be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

A member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or may act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

In some embodiments, the level of PARP in a sample from a patient is compared to predetermined standard sample. The sample from the patient is typically from a diseased tissue, such as cancer cells or tissues. The standard sample can be from the same patient or from a different subject. The standard sample is typically a normal, non-diseased sample. However, in some embodiments, such as for staging of disease or for evaluating the efficacy of treatment, the standard sample is from a diseased tissue. The standard sample can be a combination of samples from several different subjects. In some embodiments, the level of PARP from a patient is compared to a predetermined level. This pre-determined level is typically obtained from normal samples. As described herein, a “pre-determined PARP level” may be a level of PARP used to, by way of example only, evaluate a patient that may be selected for treatment, evaluate a response to a PARP inhibitor treatment, evaluate a response to a combination of a PARP inhibitor and a second therapeutic agent treatment, and/or diagnose a patient for cancer, inflammation, pain and/or related conditions. A pre-determined PARP level may be determined in populations of patients with or without cancer. The pre-determined PARP level can be a single number, equally applicable to every patient, or the pre-determined PARP level can vary according to specific subpopulations of patients. For example, men might have a different pre-determined PARP level than women; non-smokers may have a different pre-determined PARP level than smokers. Age, weight, and height of a patient may affect the pre-determined PARP level of the individual. Furthermore, the predetermined PARP level can be a level determined for each patient individually. The pre-determined PARP level can be any suitable standard. For example, the pre-determined PARP level can be obtained from the same or a different human for whom a patient selection is being assessed. In one embodiment, the predetermined PARP level can be obtained from a previous assessment of the same patient. In such a manner, the progress of the selection of the patient can be monitored over time. In addition, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s).

In some embodiments of the present invention the change of PARP from the pre-determined level is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold. In some embodiments is fold change is less than about 1, less than about 5, less than about 10, less than about 20, less than about 30, less than about 40, or less than about 50. In other embodiments, the changes in PARP level compared to a predetermined level is more than about 1, more than about 5, more than about 10, more than about 20, more than about 30, more than about 40, or more than about 50. Preferred fold changes from a predetermined level are about 0.5, about 1.0, about 1.5, about 2.0, about 2.5, and about 3.0.

The analysis of PARP levels in patients is particularly valuable and informative, as it allows the physician to more effectively select the best treatments, as well as to utilize more aggressive treatments and therapy regimens based on the up-regulated or down-regulated level of PARP. More aggressive treatment, or combination treatments and regimens, can serve to counteract poor patient prognosis and overall survival time. Armed with this information, the medical practitioner can choose to provide certain types of treatment such as treatment with PARP inhibitors, and/or more aggressive therapy.

In monitoring a patient's PARP levels, over a period of time, which may be days, weeks, months, and in some cases, years, or various intervals thereof, the patient's body fluid sample, e.g., serum or plasma, can be collected at intervals, as determined by the practitioner, such as a physician or clinician, to determine the levels of PARP, and compared to the levels in normal individuals over the course or treatment or disease. For example, patient samples can be taken and monitored every month, every two months, or combinations of one, two, or three month intervals according to the invention. In addition, the PARP levels of the patient obtained over time can be conveniently compared with each other, as well as with the PARP values, of normal controls, during the monitoring period, thereby providing the patient's own PARP values, as an internal, or personal, control for long-term PARP monitoring.

### Techniques for Analysis of PARP

The analysis of the PARP may include analysis of PARP gene expression, including an analysis of DNA, RNA, analysis of the level of PARP and/or analysis of the activity of PARP including a level of mono- and poly-ADP-ribozylation. Without limiting the scope of the present invention, any number of techniques known in the art can be employed for the analysis of PARP and they are all within the scope of the present invention. Some of the examples of such detection technique are given below but these examples are in no way limiting to the various detection techniques that can be used in the present invention.

Gene Expression Profiling: Methods of gene expression profiling include methods based on hybridization analysis of polynucleotides, polyribonucleotides methods based on sequencing of polynucleotides, polyribonucleotides and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), Comparative Genome Hybridisation (CGH), Chromatin Immunoprecipitation (ChIP), Single nucleotide polymorphism (SNP) and SNP arrays, Fluorescent in situ Hybridization (FISH), Protein binding arrays and DNA microarray (also commonly known as gene or genome chip, DNA chip, or gene array), RNA microarrays.

Reverse Transcriptase PCR (RT-PCR): One of the most sensitive and most flexible quantitative PCR-based gene expression profiling methods is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. For example, the starting material can be typically total RNA isolated from human tumors or tumor cell lines, and corresponding normal tissues or cell lines, respectively. Thus RNA can be isolated from a variety of normal and diseased cells and tissues, for example tumors, including breast, lung, colorectal, prostate, brain, liver, kidney, pancreas, spleen, thymus, testis, ovary, uterus, etc., or tumor cell lines. If the source of mRNA is a primary tumor, mRNA can be extracted, for example, from frozen or archived fixed tissues, for example paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997).

In particular, RNA isolation can be performed using purification kit, buffer set and protease from commercial manufacturers, according to the manufacturer's instructions. RNA prepared from tumor can be isolated, for example, by cesium chloride density gradient centrifugation. As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. The derived cDNA can then be used as a template in the subsequent PCR reaction.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Fluorescence Microscopy: Some embodiments of the invention include fluorescence microscopy for analysis of PARP. Fluorescence microscopy enables the molecular composition of the structures being observed to be identified through the use of fluorescently-labeled probes of high chemical specificity such as antibodies. It can be done by directly conjugating a fluorophore to a protein and introducing this back into a cell. Fluorescent analogue may behave like the native protein and can therefore serve to reveal the distribution and behavior of this protein in the cell. Along with NMR, infrared spectroscopy, circular dichroism and other techniques, protein intrinsic fluorescence decay and its associated observation of fluorescence anisotropy, collisional quenching and resonance energy transfer are techniques for protein detection. The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry. By way of example only, some of the probes are labels such as, fluorescein and its derivatives, carboxyfluoresceins, rhodamines and their derivatives, atto labels, fluorescent red and fluorescent orange: cy3/cy5 alternatives, lanthanide complexes with long lifetimes, long wavelength labels—up to 800 nm, DY cyanine labels, and phycobili proteins. By way of example only, some of the probes are conjugates such as, isothiocyanate conjugates, streptavidin conjugates, and biotin conjugates. By way of example only, some of the probes are enzyme substrates such as, fluorogenic and chromogenic substrates. By way of example only, some of the probes are fluorochromes such as, FITC (green fluorescence, excitation/emission=506/529 nm), rhodamine B (orange fluorescence, excitation/emission=560/584 nm), and nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc. Fluorescent molecular rotors are sensors of microenvironmental restriction that become fluorescent when their rotation is constrained. Few examples of molecular constraint include increased dye (aggregation), binding to antibodies, or being trapped in the polymerization of actin. IEF (isoelectric focusing) is an analytical tool for the separation of ampholytes, mainly proteins. An advantage for IEF-gel electrophoresis with fluorescent IEF-marker is the possibility to directly observe the formation of gradient. Fluorescent IEF-marker can also be detected by UV-absorption at 280 nm (20° C.).

A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used. A FACS machine originally runs cells through a capillary tube and separate cells by detecting their fluorescent intensities.

Immunoassays: Some embodiments of the invention include immunoassay for the analysis of PARP. In immunoblotting like the western blot of electrophoretically separated proteins a single protein can be identified by its antibody. Immunoassay can be competitive binding immunoassay where analyte competes with a labeled antigen for a limited pool of antibody molecules (e.g. radioimmunoassay, EMIT). Immunoassay can be non-competitive where antibody is present in excess and is labeled. As analyte antigen complex is increased, the amount of labeled antibody-antigen complex may also increase (e.g. ELISA). Antibodies can be polyclonal if produced by antigen injection into an experimental animal, or monoclonal if produced by cell fusion and cell culture techniques. In immunoassay, the antibody may serve as a specific reagent for the analyte antigen.

Without limiting the scope and content of the present invention, some of the types of immunoassays are, by way of example only, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). These techniques can be used to detect biological substances in the nasal specimen. The antibodies—either used as primary or secondary ones—can be labeled with radioisotopes (e.g. 125I), fluorescent dyes (e.g. FITC) or enzymes (e.g. HRP or AP) which may catalyse fluorogenic or luminogenic reactions.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself. This can be achieved by coupling a spacer molecule (e.g., 6-nitrohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments of the invention include ELISA to analyze PARP. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used. The test may require the antibodies to be fixed to a solid surface, such as the inner surface of a test tube, and a preparation of the same antibodies coupled to an enzyme. The enzyme may be one (e.g., β-galactosidase) that produces a colored product from a colorless substrate. The test, for example, may be performed by filling the tube with the antigen solution (e.g., protein) to be assayed. Any antigen molecule present may bind to the immobilized antibody molecules. The antibody-enzyme conjugate may be added to the reaction mixture. The antibody part of the conjugate binds to any antigen molecules that are bound previously, creating an antibody-antigen-antibody “sandwich”. After washing away any unbound conjugate, the substrate solution may be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced is proportional to the amount of enzyme-labeled antibodies bound (and thus to the concentration of the antibodies being assayed).

Some embodiments of the invention include radioimmunoassays to analyze PARP. Radioactive isotopes can be used to study in vivo metabolism, distribution, and binding of small amount of compounds. Radioactive isotopes of ¹H, ¹²C, ³¹P, ³²S, and ¹²⁷I in body are used such as ³H, ¹⁴C, ³²P, ³⁵S, and ¹²⁵I. In receptor fixation method in 96 well plates, receptors may be fixed in each well by using antibody or chemical methods and radioactive labeled ligands may be added to each well to induce binding. Unbound ligands may be washed out and then the standard can be determined by quantitative analysis of radioactivity of bound ligands or that of washed-out ligands. Then, addition of screening target compounds may induce competitive binding reaction with receptors. If the compounds show higher affinity to receptors than standard radioactive ligands, most of radioactive ligands would not bind to receptors and may be left in solution. Therefore, by analyzing quantity of bound radioactive ligands (or washed-out ligands), testing compounds' affinity to receptors can be indicated.

The filter membrane method may be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Some embodiments of the invention include fluorescence immunoassays for the analysis of PARP. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogenous or homogenous. Heterogenous immunoassays comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody is used). Homogenous immunoassays comprise no physical separation. Double-antibody fluorophore labeled antigen participates in an equilibrium reaction with antibodies directed against both the antigen and the fluorophore. Labeled and unlabeled antigen may compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that selectively measures fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody is adhered to a solid phase and a scanning probe microscope is utilized to detect antigen/antibody complexes which may be present on the surface of the solid phase. The use of scanning tunneling microscopy eliminates the need for labels which normally is utilized in many immunoassay systems to detect antigen/antibody complexes.

Protein identification methods: By way of example only, protein identification methods include low-throughput sequencing through Edman degradation, mass spectrometry techniques, peptide mass fingerprinting, de novo sequencing, and antibody-based assays. The protein quantification assays include fluorescent dye gel staining, tagging or chemical modification methods (i.e. isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure include x-ray crystallography and NMR spectroscopy. Characteristics indicative of the three-dimensional structure of proteins can be probed with mass spectrometry. By using chemical crosslinking to couple parts of the protein that are close in space, but far apart in sequence, information about the overall structure can be inferred. By following the exchange of amide protons with deuterium from the solvent, it is possible to probe the solvent accessibility of various parts of the protein.

In one embodiment, fluorescence-activated cell-sorting (FACS) is used to identify PARP expressing cells. FACS is a specialised type of flow cytometry. It provides a method for sorting a heterogenous mixture of biological cells into two or more containers, one cell at a time, based upon the specific light scattering and fluorescent characteristics of each cell. It provides quantitative recording of fluorescent signals from individual cells as well as physical separation of cells of particular interest. In yet another embodiment, microfluidic based devices are used to evaluate PARP expression.

Mass spectrometry can also be used to characterize PARP from patient samples. The two methods for ionization of whole proteins are electrospray ionization (ESI) and matrix-assisted laser desorption/ionization (MALDI). In the first, intact proteins are ionized by either of the two techniques described above, and then introduced to a mass analyser. In the second, proteins are enzymatically digested into smaller peptides using an agent such as trypsin or pepsin. Other proteolytic digest agents are also used. The collection of peptide products are then introduced to the mass analyser. This is often referred to as the “bottom-up” approach of protein analysis.

Whole protein mass analysis is conducted using either time-of-flight (TOF) MS, or Fourier transform ion cyclotron resonance (FT-ICR). The instrument used for peptide mass analysis is the quadrupole ion trap. Multiple stage quadrupole-time-of-flight and MALDI time-of-flight instruments also find use in this application.

Two methods used to fractionate proteins, or their peptide products from an enzymatic digestion. The first method fractionates whole proteins and is called two-dimensional gel electrophoresis. The second method, high performance liquid chromatography is used to fractionate peptides after enzymatic digestion. In some situations, it may be necessary to combine both of these techniques.

There are two ways mass spectroscopy can be used to identify proteins. Peptide mass uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein is present in the original sample.

Tandem MS is also a method for identifying proteins. Collision-induced dissociation is used in mainstream applications to generate a set of fragments from a specific peptide ion. The fragmentation process primarily gives rise to cleavage products that break along peptide bonds.

A number of different algorithmic approaches have been described to identify peptides and proteins from tandem mass spectrometry (MS/MS), peptide de novo sequencing and sequence tag based searching. One option that combines a comprehensive range of data analysis features is PEAKS. Other existing mass spec analysis software include: Peptide fragment fingerprinting SEQUEST, Mascot, OMSSA and X!Tandem).

Proteins can also be quantified by mass spectrometry. Typically, stable (e.g. non-radioactive) heavier isotopes of carbon (C13) or nitrogen (N15) are incorporated into one sample while the other one is labelled with corresponding light isotopes (e.g. C12 and N14). The two samples are mixed before the analysis. Peptides derived from the different samples can be distinguished due to their mass difference. The ratio of their peak intensities corresponds to the relative abundance ratio of the peptides (and proteins). The methods for isotope labelling are SILAC (stable isotope labelling with amino acids in cell culture), trypsin-catalyzed O18 labeling, ICAT (isotope coded affinity tagging), ITRAQ (isotope tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument.

N-terminal sequencing aids in the identification of unknown proteins, confirm recombinant protein identity and fidelity (reading frame, translation start point, etc.), aid the interpretation of NMR and crystallographic data, demonstrate degrees of identity between proteins, or provide data for the design of synthetic peptides for antibody generation, etc. N-terminal sequencing utilises the Edman degradative chemistry, sequentially removing amino acid residues from the N-terminus of the protein and identifying them by reverse-phase HPLC. Sensitivity can be at the level of 100s femtomoles and long sequence reads (20-40 residues) can often be obtained from a few 10s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g. using cyanogen bromide) may allow a mixture of amino acids to be generated in each cycle of the instrument, which can be subjected to database analysis in order to interpret meaningful sequence information. C-terminal sequencing is a post-translational modification, affecting the structure and activity of a protein. Various disease situations can be associated with impaired protein processing and C-terminal sequencing provides an additional tool for the investigation of protein structure and processing mechanisms.

### EXAMPLES

### Example 1

### PARP1 Expression in Uterine, Endometrial and Ovarian Cancers

Previous studies have shown increased PARP activity in ovarian cancers, hepatocellular carcinomas, and rectal tumors, compared with normal healthy control tissues, as well as in human peripheral blood lymphocytes from leukemia patients (Yalcintepe L, et. al. Braz J Med Biol Res 2005; 38:361-5. SinghN. et. al. Cancer Lett 1991; 58:131-5; Nomura F, et. al. J Gastroenterol Hepatol 2000; 15:529-35). This invention uses the gene expression databases to examine PARP1 gene regulation in more than 2000 primary malignant and normal human tissues.

### Tissue Samples

Specimens are harvested as part of a normal surgical procedure and flash frozen within 30 minutes of resection. Internal pathology review and confirmation are performed on samples subjected to analysis. Hematoxylin and eosin (H&E)-stained glass slides generated from adjacent tissues are used to confirm and classify diagnostic categories and to evaluate neoplastic cellularity. Expression of ER, PR, and HER2 is determined using immunohistochemistry and fluorescence in situ hybridization. These results, as well as attendant pathology and clinical data, are annotated with sample inventory and management databases (Ascenta, BioExpress databases; GeneLogic, Inc., Gaithersburg, Md.).

### RNA Extraction and Expression Profiling

RNA extraction and hybridization are performed as described by Hansel et al. Array data quality is evaluated using array high throughput application (Ascenta, Bioexpress Gene Logic, Gaithersburg Md. and Affymetrix, Santa Clara, Calif.), which assesses the data against multiple objective standards including 5′/3′ GAPDH ratio, signal/noise ratio, and background as well as other additional metrics. GeneChip analysis is performed with Affymetrix Microarray Analysis Suite version 5.0, Data Mining Tool 2.0, and Microarray database software (Affymetrix, Santa Clara, Calif.). All of the genes represented on the GeneChip are globally normalized and scaled to a signal intensity of 100.

### Microarray Data Analysis

Pathologically normal tissue samples are used to determine baseline expression of the PARP1 mRNA. The mean and 90%, 95%, 99%, and 99.9% upper confidence limits (UCLs) for an individual predicted value are calculated. Because we are assessing the likelihood that individual samples external to the normal set are within the baseline distribution, the prediction interval, rather than the confidence interval for the mean, is selected to estimate the expected range for future individual measurements. The prediction interval is defined by the formula, X̅±AS{square root over ((1+(1/n))}, where X̅ is the mean of the normal breast samples, S is the standard deviation, n is the sample size, and A is the 100(1−(p/2))^{th} percentile of the Student's t-distribution with n−1 degrees of freedom.

Pathologically normal tissue samples is used to determine baseline expression of the PARP1. Samples are grouped into various subcategories according to characteristics including tumor stage, smoking status, CA125 status, or age. Each tumor sample is evaluated according to 90%, 95%, 99%, or 99.9% UCLs Analysis is performed using SAS v8.2 for Windows (www.sas.com).

Pearson's correlations are calculated for 11 probe sets as compared to PARP1. Correlations are based on the complete set of 194 samples. The Pearson's product-moment correlation is defined by the formula,

$${r}_{xy} = \frac{\sum ( {x}_{i} - \overline{x} ) ( {y}_{i} - \overline{y} )}{\sqrt{{Σ ( {x}_{i} - \overline{x} )}^{2} \sum {( {y}_{i} - \overline{y} )}^{2}}} ,$$

where X̅ is the mean of the PARP1 probe set and Y̅ is the mean of the probe set to which PARP1 is being correlated. Statistical significance is determined by the formula,

$$\frac{{( n - 2 )}^{1 / 2} r}{{( 1 - {r}^{2} )}^{1 / 2}} ,$$

where r is the correlation and n is the number of samples. The resultant value is assumed to have at distribution with n−2 degrees of freedom.

### Multiplex Reverse Transcriptase-Polymerase Chain Reaction (RT-PCR):

Multiplex RT-PCR is performed using 25 ng of total RNA of each sample as previously described (Khan et al., 2007). The multiplex assay used for this study is designed to detect RNA from formalin fixed paraffin embedded (FFPE) samples or from frozen tissues. The concentration of the RNA is determined using the RiboGreen RNA Quantitation Kit (Invitrogen) with Wallac Victo r2 1420 Multilabel Counter. A sample of RNA from each sample is analyzed on an Agilent Bioanalyzer following instructions of Agilent 2100 Bioanalyzer. Reverse transcription (RT) reactions are carried out as previously described with the Applied Biosystems 9700. PCR reactions are carried out on each cDNA with the Applied Biosystems 9700. RT reactions are spiked with Kanamycin RNA to monitor efficiency of the RT and PCR reactions. Controls used included positive control RNA, a no template control, and a no reverse transcriptase control. PCR reactions are analyzed by capillary electrophoresis. The fluorescently labeled PCR reactions are diluted, combined with Genome Lab size standard-400 (Beckman-Coulter), denatured, and assayed with the CEQ 8800 Genetic Analysis System. The expression of each target gene relative to the expression of β-glucuronidase (GUSB) within the same reaction is reported as the mean and standard deviation of 3 independent assessments for each sample.

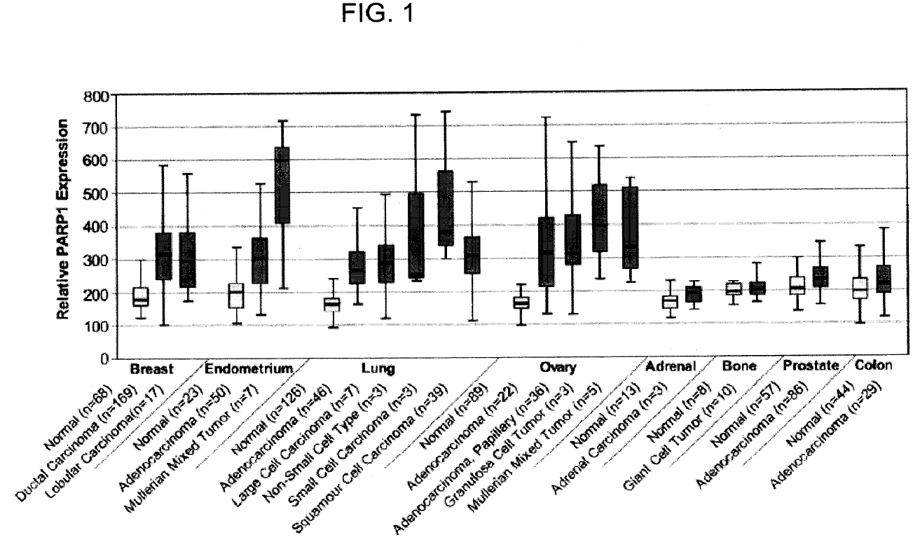

While PARP1 expression and activity is very low and uniform across the majority of normal human tissues and organs, it is upregulated in selected tumor cells and primary human malignancies, with the most striking differences found in breast, ovarian, lung, and uterine cancers (FIG. 1).

### Example 2

### Nonclinical Pharmacology in Ovarian Carcinoma Tumor Model

4-iodo-3-nitrobenzamide (BA) is active against a broad range of cancer cells in culture, including drug resistant cell lines. In in vitro studies, BA inhibits the proliferation of a variety of human tumor cells including breast, colon, prostate, cervix, lung, and ovarian cancers.

### Mice

Female CB.17 SCID mice (Charles River) are 8-11 weeks old, and have a body weight (BW) range of 12.6-23.0 g on D1 of the study. Female athymic mice (nu/nu, Harlan) are 11 weeks old, and have a body weight (BW) range of 18.9-28.4 g on D1 of the study. The animals are fed ad libitum water (reverse osmosis, 1 ppm C1) and NIH 31 Modified and Irradiated Lab Diet® consisting of 18.0% crude protein, 5.0% crude fat, and 5.0% crude fiber. The mice are housed on irradiated ALPHA-dri® Bed-o-cobs® Laboratory Animal Bedding in static microisolators on a 12-hour light cycle at 21-22° C. (70-72° F.) and 40-60% humidity in the laboratory accredited by Association for Assessment and Accreditation of Laboratory International, which assures compliance with accepted standards for the care and use of laboratory animals.

### Tumor Implantation

The human OVCAR-3 (NIH-OVCAR-3) ovarian adenocarcinoma utilized in the study is maintained in athymic nude mice by serial engraftment. The human SW620 colon adenocarcinoma utilized in the study is maintained in nude mice by serial engraftment. A tumor fragment (1 mm³) is implanted s.c. into the right flank of each test mouse. Tumors are monitored twice weekly and then daily as their volumes approached 80-120 mm³. On D1 of the study, animals are sorted into treatment groups with tumor sizes of 63-221 mm³ and group mean tumor sizes of ˜105 mm³.

Tumor weight may be estimated with the assumption that 1 mg is equivalent to 1 mm³ of tumor volume.
Tumor size, in mm³, was calculated from:

$$Tumor Volume = \frac{{w}^{2} \times I}{2}$$

### Treatment

Mice are sorted into groups (n=10) and treated in accordance with the protocol. Oral group receives BA p.o. (orally) twice daily from D1 p.m. until D68 a.m. (b.i.d. to end, i.e. twice daily dosing for the duration of the study). Alzet model osmotic pumps are implanted on Days 1, 15, and 29. The pumps are pre-warmed for 1 hour at 37° C., and then implanted subcutaneously (s.c.) in the left flanks of isofluoraneanesthetized mice. Each pump delivers a total dose of 25 mg/kg/week of BA over 14 days. BA is administrated intraperitoneally (i.p.) 15 mg/kg respectively twice weekly.

### Endpoint

Tumors are calipered twice weekly for the duration of the study. Each animal is euthanized when its neoplasm reached the predetermined endpoint size (1,000 mm³). The time to endpoint (TTE) for each mouse is calculated by the following equation:

$$T T E = \frac{{log}_{10} ( endpoint volume ) - b}{m}$$

where TTE is expressed in days, endpoint volume is in mm³, b is the intercept, and m is the slope of the line obtained by linear regression of a log-transformed tumor growth data set. The data set is comprised of the first observation that exceeds the study endpoint volume and the three consecutive observations that immediately precede the attainment of the endpoint volume. The calculated TTE is usually less than the day on which an animal is euthanized for tumor size. Animals that do not reach the endpoint are euthanized at the end of the study, and assigned a TTE value equal to the last day (68 days). Treatment efficacy is determined from tumor growth delay (TGD), which is defined as the increase in the median TTE for a treatment group compared to the control group: TGD=T−C, (i.e. difference between the median TTE values of Treated and Control mice) expressed in days, or as a percentage of the median TTE of the control group:

$$\% T G D = \frac{T - C}{C} \times 100$$

where:
T=median TTE for a treatment group,
C=median TTE for control Group 1.

### Preparation of Peripheral Blood Lymphocyte and Tumor Samples

Whole blood is collected into EDTA vacutainers and human PBMCs are obtained by BD Vacutainer™ CPT™ Cell Preparation kit according to the manufacturer's instructions (BD Vacutainer™, REF 362760). Tumor samples are collected in a sterile container and placed immediately on ice. Within 30 minutes, tumor samples are snap-frozen in liquid nitrogen and stored at −80° C. until homogenized for analysis. The specimen is defrosted on ice and the wet weight is documented. The tissue is homogenized using isotonic buffer [7 mmol/L HEPES, 26 mmol/L KCl, 0.1 mmol/L dextran, 0.4 mmol/L EGTA, 0.5 mmol/L MgCl2, 45 mmol/L sucrose (pH 7.8)]. The homogenate is kept on ice throughout the process, and homogenization is done in 10-second bursts to prevent undue warming of the sample. Unless assayed on the day of homogenization, samples are refrozen to −80° C. and stored at this temperature until analyzed.

### Poly(ADP-Ribose) Polymerase Assay Procedure

Cell preparations are defrosted rapidly at room temperature and washed twice in ice-cold PBS. The cell pellets are resuspended in 0.15 mg/mL digitonin to a density of 1×10⁶ to 2×10⁶ cells/mL for 5 minutes to permeabilize the cells (verified by trypan blue staining), following which 9 volumes of ice-cold isotonic buffer are added and the sample is placed on ice. Maximally stimulated PARP activity is measured in replicate samples of 20,000 cells in a reaction mixture containing 350 mmol/L NAD+ and 10 mg/mL oligonucleotide in a reaction buffer of 100 mmol/L Tris-HCl, 120 mmol/L MgCl2 (pH 7.8) in a final volume of 100 mL as described previously (24) at 26° C. in an oscillating water bath. The reaction is stopped after 6 minutes by the addition of excess PARP inhibitor (400 μL of 12.5 μmol/L AG014699) and the cells are blotted onto a nitrocellulose membrane (Hybond-N, Amersham) using a 24-well manifold. Purified PAR standards are loaded onto each membrane (0-25 μmol monomer equivalent) to generate a standard curve and allow quantification. Overnight incubation with the primary antibody (1:500 in PBS+0.05% Tween 20+5% milk powder) at 4° C. is followed by two washes in PBS-T (PBS+0.05% Tween 20) and then incubation in secondary antibody (1:1,000 in PBS+0.05% Tween 20+5% milk powder) for 1 hour at room temperature. The incubated membrane is washed frequently with PBS over the course of 1 hour and then exposed for 1 minute to enhanced chemiluminescence reaction solution as supplied by the manufacturer. Chemiluminesence detected during a 5-minute exposure is measured using a Fuji LAS3000 UV Illuminator (Raytek, Sheffield, United Kingdom) and digitized using the imaging software (Fuji LAS Image version 1.1, Raytek). The acquired image is analyzed using Aida Image Analyzer (version 3.28.001), and results are expressed in LAU/mm². Three background areas on the exposed blot are measured and the mean of the background signal from the membrane is subtracted from all results. The PAR polymer standard curve is analyzed using an unweighted one-site binding nonlinear regression model and unknowns read off the standard curve so generated. Results are then expressed relative to the number of cells loaded. Triplicate quality control samples of 5,000 L1210 cells are run with each assay, all samples from one patient being analyzed on the same blot. Tumor homogenates are assayed in a similar manner; however, the homogenization process introduces sufficient DNA damage to maximally stimulate PARP activity and oligonucleotide is not therefore required. The protein concentration of the homogenate is measured using the BCA protein assay and Titertek Multiscan MCC/340 plate reader. Results are expressed in terms of pmol PAR formed/mg protein.

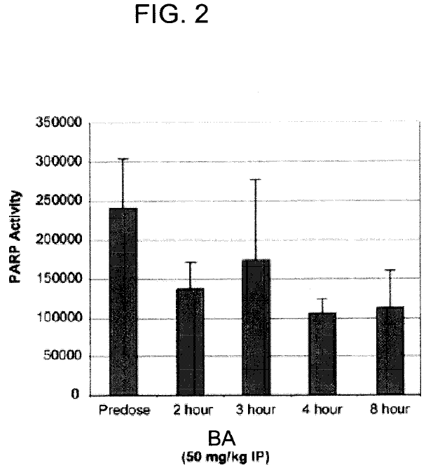

In vivo studies have demonstrated PARP inhibition by BA in animal models of cancer. For example, evaluation of tissue samples obtained from a human ovarian adenocarcinoma OVCAR-3 xenograft model in SCID mice after a single dose of BA demonstrates an inhibitory effect of BA on PARP activity that is sustained for at least 8 hours of observation (FIG. 2).

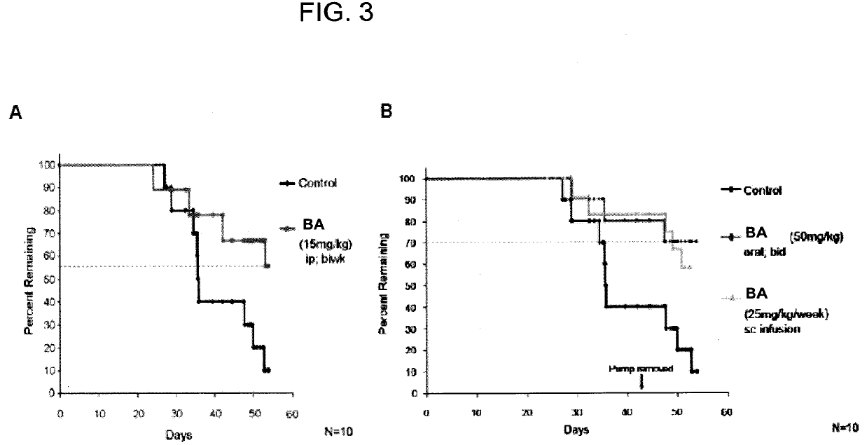

Early in vivo efficacy studies using the OVCAR-3 xenograft model in SCID mice have shown that BA significantly inhibits tumor growth. Treatment of these mice with BA via different routes of administration improves survival, compared with the untreated control (FIG. 3).

### Example 3

### Phase IB Study of BA in Combination with Chemotherapy in Patients with Advanced Solid Tumors

A Phase 1 b, open-label, dose escalation study evaluates the safety of 4-iodo-3-nitrobenzamide (BA) (2.0, 2.8, 4.0, 5.6, 8.0, and 11.2 mg/kg) in combination with chemotherapeutic regimens (topotecan, gemcitabine, temozolomide, and carboplatin+paclitaxel) in subjects with advanced solid tumors including ovarian tumors. The dose-escalation phase of the study has been completed, and well tolerated combinations of BA and cytotoxic chemotherapy have been identified. The protocol has been amended to evaluate BA in combination with chemotherapy in specific tumor types.

### Rationale

Topotecan targets topoisomerase I, which plays a critical role in DNA replication, transcription, and Recombination. Topotecan selectively stabilizes topoisomerase I-DNA covalent complexes, inhibiting re-ligation of topoisomerase I-mediated single-strand DNA breaks and producing lethal double-strand DNA breaks. Poly(ADP- Ribose) Polymerase-1 (PARP-1) interacts with topoisomerase I and increases tumor sensitivity to topoisomerase 1 inhibitors. Preclinical studies show that the PARP1 inhibitor BA potentiates the antitumor activity of topotecan. PARP1 is signi_cantly up-regulated in human primary ovarian tumors.

### Study Design:

BA plus cytotoxic chemotherapy (CTX)

CTX Dosing:
Topotecan: 1.5 mg/m2 or 1.1 mg/m2 QD for 5 days of 21 day cycle
Temozolomide: 75 mg/m2 P.O. QD for 21 days of 28 day cycle
Gemcitabine: 1000 mg/m2 as 30 min infusion QW; 7 of 8 weeks; initial 28 days for safety evaluation
Carboplatin/Paclitaxel: C=AUC of 6; P×1=200 mg/m2; both on day 1 of 21 day cycle

BA Dosing:
Twice weekly; i.v. infusion
Standard 3+3 design for BA dose escalation
Dose levels studied: 2.0, 2.8, 4.0, 5.6, 8.0, and up to 11.2 mg/kg

### Study Endpoints:

Safety, tolerability and MTD of each combination

Clinical response via RECIST every 2 cycles

### General Eligibility:

Subjects_{—} 18 years old with a refractory, advanced solid tumor, ECOG PS of <=2, and adequate hematological, renal, and hepatic function

No restriction on number of prior chemotherapeutic regimens

### Efficacy

In terms of efficacy, 53 of 66 subjects demonstrate some clinical benefit (Table 1).

TABLE 1
Clinical Results

| | Average # | | SD ≧ 6 | SD ≧ 2 |
|---|---|---|---|---|
| Study Arm (N) | of cycles | CR + PR | Cycles | Cycles |
| Topotecan (14) | 2.9 | 1 | 2 | 7 |
| Temozolomide (17) | 2.4 | 1 | 0 | 13 |
| Gemcitabine (22) | 3.4 | 3 | 1 | 12 |
| Carbo/Taxol (13) | 4.6 | 2 | 1 | 10 |
| Total (66) | 3.3 | 7 | 4 | 42 |

1 CR - ovarian; 6 PR - 2 breast, 1 uterine, 1 ovarian, 1 renal, 1 sarcoma; 4 SD >= 6 cycles - 1 adenocarcinosarcoma, 1 ACUP, 2 sarcoma; 42 SD >= 2 cycles-multiple tumor types

### Ovarian Cancer Patient Response

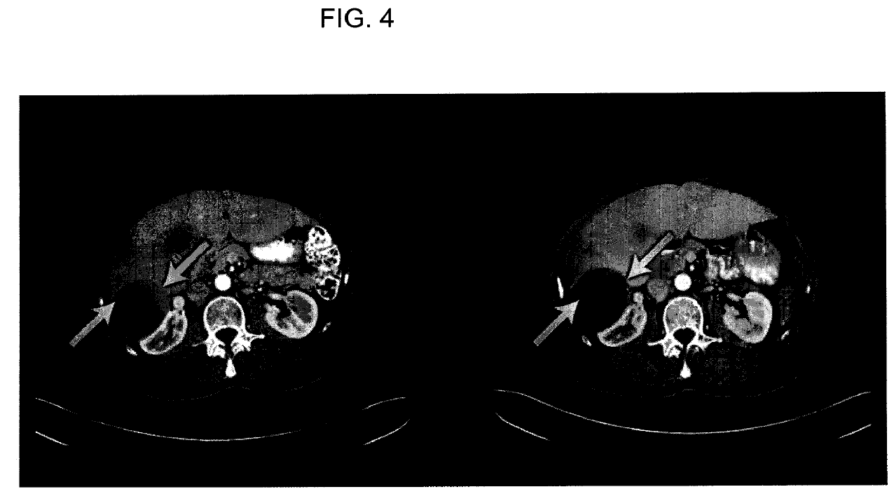

As shown in FIG. 4, a patient with advanced ovarian cancer has a partial response after 4 cycles of BA in a combination with topotecan. Liver lesion (target lesion) shrinks from 4.6 cm to 1.5 cm. CA 27-29 biomarker also reduces from >300 to <200.

### Preparation of Peripheral Blood Lymphocyte and Tumor Samples

Whole blood is collected into EDTA vacutainers and human PBMCs are obtained by BD Vacutainer™ CPT™ Cell Preparation kit according to the manufacturer's instructions (BD Vacutainer™, REF 362760). Tumor samples are collected in a sterile container and placed immediately on ice. Within 30 minutes, tumor samples are snap-frozen in liquid nitrogen and stored at −80° C. until homogenized for analysis. The specimen is defrosted on ice and the wet weight is documented. The tissue is homogenized using isotonic buffer [7 mmol/L HEPES, 26 mmol/L KCl, 0.1 mmol/L dextran, 0.4 mmol/L EGTA, 0.5 mmol/L MgCl2, 45 mmol/L sucrose (pH 7.8)]. The homogenate is kept on ice throughout the process, and homogenization is done in 10-second bursts to prevent undue warming of the sample. Unless assayed on the day of homogenization, samples are refrozen to −80° C. and stored at this temperature until analyzed.

### Poly(ADP-Ribose) Polymerase Assay Procedure

Cell preparations are defrosted rapidly at room temperature and washed twice in ice-cold PBS. The cell pellets are resuspended in 0.15 mg/mL digitonin to a density of 1×10⁶ to 2×10⁶ cells/mL for 5 minutes to permeabilize the cells (verified by trypan blue staining), following which 9 volumes of ice-cold isotonic buffer are added and the sample is placed on ice. Maximally stimulated PARP activity is measured in replicate samples of 20,000 cells in a reaction mixture containing 350 mmol/L NAD+ and 10 mg/mL oligonucleotide in a reaction buffer of 100 mmol/L Tris-HCl, 120 mmol/L MgCl₂ (pH 7.8) in a final volume of 100 mL as described previously (24) at 26° C. in an oscillating water bath. The reaction is stopped after 6 minutes by the addition of excess PARP inhibitor (400 μl of 12.5 μmol/L AG014699) and the cells are blotted onto a nitrocellulose membrane (Hybond-N, Amersham) using a 24-well manifold. Purified PAR standards are loaded onto each membrane (0-25 pmol monomer equivalent) to generate a standard curve and allow quantification. Overnight incubation with the primary antibody (1:500 in PBS+0.05% Tween 20+5% milk powder) at 4° C. is followed by two washes in PBS-T (PBS+0.05% Tween 20) and then incubation in secondary antibody (1:1,000 in PBS+0.05% Tween 20+5% milk powder) for 1 hour at room temperature. The incubated membrane is washed frequently with PBS over the course of 1 hour and then exposed for 1 minute to enhanced chemiluminescence reaction solution as supplied by the manufacturer. Chemiluminesence detected during a 5-minute exposure is measured using a Fuji LAS3000 UV Illuminator (Raytek, Sheffield, United Kingdom) and digitized using the imaging software (Fuji LAS Image version 1.1, Raytek). The acquired image is analyzed using Aida Image Analyzer (version 3.28.001), and results are expressed in LAU/mm2. Three background areas on the exposed blot are measured and the mean of the background signal from the membrane is subtracted from all results. The PAR polymer standard curve is analyzed using an unweighted one-site binding nonlinear regression model and unknowns read off the standard curve so generated. Results are then expressed relative to the number of cells loaded. Triplicate quality control samples of 5,000 L1210 cells are run with each assay, all samples from one patient being analyzed on the same blot. Tumor homogenates are assayed in a similar manner; however, the homogenization process introduces sufficient DNA damage to maximally stimulate PARP activity and oligonucleotide is not therefore required. The protein concentration of the homogenate is measured using the BCA protein assay and Titertek Multiscan MCC/340 plate reader. Results are expressed in terms of pmol PAR formed/mg protein.

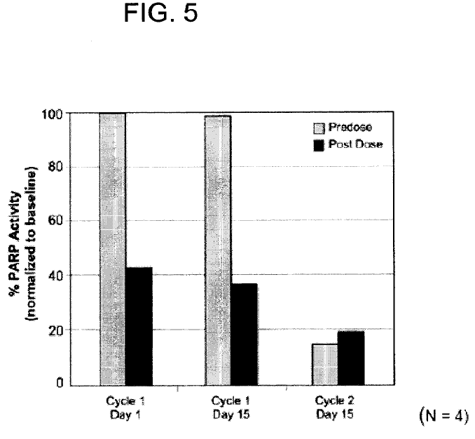

Evaluation of peripheral blood mononuclear cells (PBMCs) from patients shows significant and prolonged PARP inhibition after multiple dosing with BA doses of 2.8 mg/kg or higher (FIG. 5).

Well tolerated combinations of BA and cytotoxic chemotherapy are identified. Any toxicities observed are consistent with known and expected side effects of each chemotherapeutic regimen. There is no evidence that the addition of BA to any tested cytotoxic regimen either potentiates known toxicities or increases the frequency of their expected toxicities. A biologically relevant dose (2.8 mg/kg) that elicits significant and sustained PARP inhibition at effective preclinical blood concentrations is identified. Approximately 80% of subjects demonstrate evidence of stable disease for 2 cycles of treatment or more, indicating potential clinical benefit. The observed pattern of tumor response is consistent with PARP expression and/or synergy with chemotherapeutic agents.

### Example 4

### Treatment of Advanced, Persistent or Recurrent Uterine Carcinosarcoma with BA

A multi-center, open-label, randomized study to demonstrate the therapeutic effectiveness in the treatment of advanced, persistent or recurrent uterine carcinosarcoma with 4-iodo-3-nitrobenzamide (BA) is conducted.

Study Objectives: The Primary Objectives of this Study are as Follows:

Clinical Benefit Rate (CBR=CR+PR+SD≧6 months): Determine that BA will produce a CBR of 30% or greater as compared to the CBR of 45% associated with treatment with gemcitabine and carboplatin.
To further study the safety and tolerability of BA
The secondary objectives of this study are as follows:
Overall Response Rate (ORR)
Progression-free survival (PFS)
Evaluation of the toxicity associated with each arm
The exploratory objectives of this study are as follows:
To characterized the inhibition of PARP activity by BA
To characterize PARP activity in historic tumor tissue samples
To study the status of BRCA in advanced, persistent or recurrent uterine cancer
To study the response in subjects with cancer and known BRCA mutations compared to subjects without these mutations

Study Design: An open label, 2-arm randomized, safety and efficacy study in which up to 90 patients (45 in each arm) will be randomized to either:
Study Arm 1: Gemcitabine (1000 mg/m²; 30 min IV infusion) and Carboplatin (AUC 2; 60 min IV infusion) on days 1 and 8 of a 21-day cycle; or
Study Arm 2: 4-iodo-3-nitrobenzamide (4 mg/kg 1 hour IV infusion) on days 1, 4, 8 and 11 of each 21-day cycle
Patients randomized to Study Arm 2 will be discontinued from the study at the time of disease progression
Crossover: Patients randomized to Study Arm 1 may cross over to receive continued treatment with gemcitabine/carboplatin in combination with 4-iodo-3-nitrobenzamide at the time of disease progression
Sample Size: Up to 90 subjects, up to 45 in each arm participate in the study. Subjects will be randomized, up to 45 in each of Arm-1 or Arm-2.

Subject Population:
Inclusion Criteria:
At least 18 years of age
Advanced, persistent or recurrent uterine carcinosarcoma with measurable disease by RECIST criteria
0-2 prior chemotherapy regimens in the metastatic setting. Prior adjuvant/neoadjuvant therapy is allowed.
Histology documents (either primary or metastatic site) uterine cancer that is ER-negative, PR-negative and HER-2 non-overexpressing by immunohistochemistry (0, 1) or non-gene amplified by FISH performed upon the primary tumor or metastatic lesion.
Completion of prior chemotherapy at least 3 weeks prior to study entry.
Patients may have received therapy in the adjuvant or metastatic setting, however if taking bisphosphonates, bone lesions may not be used for progression or response.
Radiation therapy must be completed at least 2 weeks prior to study entry, and radiated lesions may not serve as measurable disease.
Patients may have CNS metastases if stable (no evidence of progression) for at least 3 months after local therapy
ECOG performance status 0-1
Adequate organ function defined as: ANC greater than or equal to 1,5000/mm³, platelets greater than or equal to 100,000/mm³, creatinine clearance greater than 50 mL/min, ALT and AST lower than 2.5× upper limit of normal (ULN) (Or lower than 5×ULN in case of liver metastases); total biliruibin lower than 1.5 mg/dL.
Tissue block available for PARP studies is recommended, although will not exclude patients from participating
Pregnant or lactating women will be excluded. Women of child bearing potential must have documented negative pregnancy test within two weeks of study entry and agree to acceptable birth control during the duration of the study therapy
Signed, IRB approved written informed consent

Exclusion Criteria:
Lesions identifiable only by PET
More than 2 prior chemotherapy regimens (including adjuvant). Sequential regimens such as AC-paclitaxel are considered one regimen.
Has received prior treatment with gemcitabine, carboplatin, cisplatin or 4-iodo-3-nitrobenzamide.
Major medical conditions that might affect study participation (uncontrolled pulmonary, renal or hepatic dysfunction, uncontrolled infection).
Significant history of uncontrolled cardiac disease; i.e., uncontrolled hypertension, unstable angina, recent myocardial infarction (within prior 6 months), uncontrolled congestive heart failure, and cardiomyopathy that is either symptomatic or asymptomatic but with decreased ejection fraction lower than 45%.
Other significant comorbid condition which the investigator feels might compromise effective and safe participation in the study.
Subject enrolled in another investigational device of drug trial, or is receiving other investigational agents
Concurrent or prior (within 7 days of study day 1) anticoagulation therapy (low dose for port maintenance allowed)
Specified concomitant medications
Concurrent radiation therapy is not permitted throughout the course of the study
Inability to comply with the requirements of the study
Screening tests and evaluation will be performed only after a signed, written Institutional Review Board (IRB) approved informed consent is obtained from each subject. Procedures will be performed within 14 days of dosing (day 1) unless otherwise noted.

Clinical evaluation: Complete history, physical examination, ECOG status, height, weight, vital signs, and documentation of concomitant medications.

Laboratory studies: Hematology (with differential, reticulocyte count, and platelets); prothrombin time (PT) and partial thromboplastin time (PTT); comprehensive chemistry panel (sodium, potassium, chloride, CO₂, creatinine, calcium, phosphorus, magnesium, BUN, uric acid, albunin, AST, ALT, alkaline phosphatase, total bilirubin, and cholesterol, HDL and LDL), urinalyisis with microscopic examination, PARP inhibition in PBMCs, serum or urine pregnancy test for women of child bearing potential. BRCA profiling will be obtained if a separate informed consent is signed. This information may be also pulled from a subject's medical history. CLincial staging: imaging for measurable disease by computed tomography (CT) or magnetic resonance (MRI).

Treatment: Eligible patients will be enrolled in the study and randomized to either Arm 1 or Arm 2:
Study Arm 1: Gemcitabine (1000 mg/m²; 30 min IV infusion) and Carboplatin (AUC 2; 60 min IV infusion) on days 1 and 8 of a 21-day cycle; or
Study Arm 2: 4-iodo-3-nitrobenzamide (4 mg/kg, 1 hour IV infusion) on days 1, 4, 8 and 11 of each 21-day cycle.
Crossover: Patients randomized to study arm 1 may crossover to receive continued treatment with gemcitabine/carboplatin in combination with 4-iodo-3-nitrobenzamide at the time of disease progression.
Pre-dose and post-dose tests will be performed as outlined in the study protocol.
Dosing for both treatment arms will be repeated in 21-day cycles.

Subjects may participate in this study until they experience a drug intolerance or disease progression or withdraw consent. Subjects that achieve a CR would receive an additional 4 cycles. Subjects that discontinue treatment before PD should undergo regular staging evaluation per protocol until time of PD. Once a subject discontinues treatment, evaluation for progression free survival and overall response rate will continue at 3-month intervals until disease progression or death.

The first scheduled tumor response measurement for measurable disease will be performed after cycle 2, and then every other cycles of therapy (approximately every 6-8 weeks) in addition to the initial staging done at baseline. Tumor response according to the modified Response Evaluation Criteria in Solid Tumors (RECIST) will be used to establish disease progression by CT or MRI (the same technique used during screening must be used).

End of Treatment: All subjects should have the end of treatment procedures as described in the protocol completed no more than 30 days after the last dose of 4-iodo-3-nitrobenzamide. Additionally, subjects will have overall tumor response assessed via clinical imaging if not done within 30 days prior to the last dose of 4-iodo-3-nitrobenzamide.

Assessment of Safety: Safety will be assessed by standard clinical and laboratory tests (hematology, blood chemistry, and urinalysis). Toxicity grade is defined by the National Cancer Institute CTCAE v3.0.

Pharmacokinetics/Pharmacodynamics

Blood samples for PK and pharmacodynamic analysis will be obtained only from subjects who are enrolled onto study arm 2 this includes crossover subjects.

PK Samples will be collected during cycle 1, pre dose and immediately at the end of infusion on days 1 and 11.

Pharmacodynamic or PARP samples will be collected during cycle 1, pre dose on days 1, 4, 8 and 1. Post dose samples only on day 1.

Sites that are unable to perform the PK or pharmacodynamic sample collection as specified will be permitted to participate in the study, and the protocol will be amended accordingly at those sites.

Efficacy: Tumors will be assessed by standard methods (eg, CT) at baseline and then approximately every 6-8 weeks thereafter in the absence of clinically evident progression of disease.

Statistical Methods

The primary objective of the study is to estimate the clinical benefit rate (CBR) in the BA arm. In each of the two arms, the primary efficacy endpoint (CBR) will be estimated, and the exact binomial 90% confidence interval will be calculated. The CBRs in the two arms will be compared using a one-sided Fisher's exact test at the 5% level of significance. Secondary and exploratory efficacy endpoints of progression-free survival and overall survival will be estimated, and 95% confidence intervals will be calculated using the Kaplan-Meier method. The distributions of progression-free survival and overall survival in the two arms will be compared using the log-rank test. Analyses of PARP inhibition data will be exploratory and descriptive in nature. For the primary safety endpoint, AEs and serious adverse events (SAEs) will be tabulated by study arm, system organ class, and preferred terms. Laboratory test results after the first cycle will be summarized with regard to shifts from baseline values.

Follow-Up: On day 90 and every 90 days (±20 days) after the last dose of study drug follow-up information will be obtained.

Laboratory assessments—Blood and urine samples for hematology, serum chemistry, and urinalysis will be prepared using standard procedures. Laboratory panels are defined as follows:

Hematology: WBC count with differential, RBC count, hemoglobin, hematocrit, and platelet count

Serum chemistry: albumin, ALP, ALT, AST, BUN, calcium, carbon dioxide, chloride, creatinine, γ-glutamyl transferase, glucose, lactate dehydrogenase, phosphorus, potassium, sodium, total bilirubin, and total protein

Urinalysis: appearance, color, pH, specific gravity, ketones, protein, glucose, bilirubin, nitrite, urobilinogen, and occult blood (microscopic examination of sediment will be performed only if the results of the urinalysis dipstick evaluation are positive)

Pharmacokinetic blood samples will be obtained only from subjects who are enrolled in study arm 2 or who crossover onto study arm 2. Samples will be collected immediately pre dose and immediately at the end of each infusion during cycle 1 on study days 1 and 11.

Biomarkers are objectively measured and evaluated indicators of normal biologic processes, pathogenic processes, or pharmacologic responses to a therapeutic intervention. In oncology, there is particular interest in the molecular changes underlying the oncogenic processes that may identify cancer subtypes, stage disease, assess the amount of tumor growth, or predict disease progression, metastasis, and responses to BA.

The functional activity of PARP before and after treatment of BA will be determined using a PARP activity assay in Peripheral Blood Mononuclear Cells (PBMCs). PBMCs will be prepared from 5 mL blood samples according to procedures described in detail in the study manual and PARP activity/inhibition will be measured.

Refer to the study manual that will be provided to each site for detailed collection, handling, and shipping procedures for all PARP samples.

A breast cancer (BRCA) gene test is a blood test to check for specific changes (mutations) in genes (BRCA1 and BRCA2) that help control normal cell growth. Women who have BRCA mutations have been shown to have between a 16% and 60% chance of developing ovarian cancer. Administration of a PARP inhibitor to women with a BRCA mutation may prove to be beneficial. This study is an initial attempt to determine any association between BRCA status and response to treatment with BA.

In order to accomplish this, BRCA status should be determined (if not already known) for all subjects. A subject will need to sign a separate informed consent form. As this is not an inclusion criteria for the study, potential subjects who do not agree to this testing will not be excluded from participating in this study for this reason alone.

In each of the two arms, the primary efficacy endpoint (CBR) will be estimated, and the exact binomial 90% confidence interval will be calculated. The CBRs in the two arms will be compared using a one-sided Fisher's exact test at the 5% level of significance. Secondary and exploratory efficacy endpoints of progression-free survival and overall survival in the two arms will be compared using the log-rank test.

Tumor response data will be reported descriptively as listings for all subjects in the safety population for purposes of determining whether BA treatment has had a measurable clinical effect (e.g. time to progression) and should be continued beyond the first 8 weeks. Response data will be categorized using the modified RECIST.

PARP inhibition analysis will be exploratory as appropriate and descriptive in nature. Statistical group comparisons for differences in PARP inhibition and any pharmacogenomic results (e.g. BRCA) from samples taken before, during and after BA treatment will be considered.

Analyses of safety will be completed for all subjects who receive at least 1 dose of BA.

BA used in the study will be formulated in a 10 mg/mL concentration containing 25% hydroxypropylbetacyclodextrin in a 10 mM phosphate buffer (pH 7.4).

### Response Evaluation Criteria in Solid Tumors (RECIST):

Eligibility

Only patients with measurable disease at baseline should be included in protocols where objective tumor response is the primary endpoint.

Measurable disease—the presence of at least one measurable lesion. If the measurable disease is restricted to a solitary lesion, its neoplastic nature should be confirmed by cytology/histology.

Measurable lesions—lesions that can be accurately measured in at least one dimension with longest diameter ≧20 mm using conventional techniques or ≧10 mm with spiral CT scan.

Non-measurable lesions—all other lesions, including small lesions (longest diameter <20 mm with conventional techniques or <10 mm with spiral CT scan), i.e., bone lesions, leptomeningeal disease, ascites, pleura/pericardial effusion, inflammatory breast disease, lymphangitis cutis/pulmonis, cystic lesions, and also abdominal masses that are not confirmed and followed by imaging techniques; and.

All measurements should be taken and recorded in metric notation, using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up.

Clinical lesions will only be considered measurable when they are superficial (e.g., skin nodules and palpable lymph nodes). For the case of skin lesions, documentation by color photography, including a ruler to estimate the size of the lesion, is recommended.

Methods of Measurement

CT and MRI are the best currently available and reproducible methods to measure target lesions selected for response assessment. Conventional CT and MRI should be performed with cuts of 10 mm or less in slice thickness contiguously. Spiral CT should be performed using a 5 mm contiguous reconstruction algorithm. This applies to tumors of the chest, abdomen and pelvis. Head and neck tumors and those of extremities usually require specific protocols.

Lesions on chest X-ray are acceptable as measurable lesions when they are clearly defined and surrounded by aerated lung. However, CT is preferable.

When the primary endpoint of the study is objective response evaluation, ultrasound (US) should not be used to measure tumor lesions. It is, however, a possible alternative to clinical measurements of superficial palpable lymph nodes, subcutaneous lesions and thyroid nodules. US might also be useful to confirm the complete disappearance of superficial lesions usually assessed by clinical examination.

The utilization of endoscopy and laparoscopy for objective tumor evaluation has not yet been fully and widely validated. Their uses in this specific context require sophisticated equipment and a high level of expertise that may only be available in some centers. Therefore, the utilization of such techniques for objective tumor response should be restricted to validation purposes in specialized centers. However, such techniques can be useful in confirming complete pathological response when biopsies are obtained.

Tumor markers alone cannot be used to assess response. If markers are initially above the upper normal limit, they must normalize for a patient to be considered in complete clinical response when all lesions have disappeared.

Cytology and histology can be used to differentiate between PR and CR in rare cases (e.g., after treatment to differentiate between residual benign lesions and residual malignant lesions in tumor types such as germ cell tumors).

Baseline Documentation of “Target” and “Non-Target” Lesions

All measurable lesions up to a maximum of five lesions per organ and 10 lesions in total, representative of all involved organs should be identified as target lesions and recorded and measured at baseline.

Target lesions should be selected on the basis of their size (lesions with the longest diameter) and their suitability for accurate repeated measurements (either by imaging techniques or clinically).

A sum of the longest diameter (LD) for all target lesions will be calculated and reported as the baseline sum LD. The baseline sum LD will be used as reference by which to characterize the objective tumor.

All other lesions (or sites of disease) should be identified as non-target lesions and should also be recorded at baseline. Measurements of these lesions are not required, but the presence or absence of each should be noted throughout follow-up.

Response Criteria

Evaluation of target lesions:
Complete Response (CR): Disappearance of all target lesions
Partial Response (PR): At least a 30% decrease in the sum of the LD of target lesions, taking as reference the baseline sum LD
Progressive Disease (PD): At least a 20% increase in the sum of the LD of target lesions, taking as reference the smallest sum LD recorded since the treatment started or the appearance of one or more new lesions
Stable Disease (SD): Neither sufficient shrinkage to qualify for PR nor sufficient increase to qualify for PD, taking as reference the smallest sum LD since the treatment started

Evaluation of non-target lesions:
Complete Response (CR): Disappearance of all non-target lesions and normalization of tumor marker level
Incomplete Response/Stable Disease (SD): Persistence of one or more non-target lesion(s) or/and maintenance of tumor marker level above the normal limits
Progressive Disease (PD): Appearance of one or more new lesions and/or unequivocal progression of existing non-target lesions (1)
Although a clear progression of “non target” lesions only is exceptional, in such circumstances, the opinion of the treating physician should prevail and the progression status should be confirmed later on by the review panel (or study chair).

Evaluation of Best Overall Response

The best overall response is the best response recorded from the start of the treatment until disease progression/recurrence (taking as reference for PD the smallest measurements recorded since the treatment started). In general, the patient's best response assignment will depend on the achievement of both measurement and confirmation criteria

| Target | | New | Overall |
|---|---|---|---|
| lesions | Non-Target lesions | Lesions | response |
| CR | CR | No | CR |
| CR | Incomplete response/SD | No | PR |
| PR | Non-PD | No | PR |
| SD | Non-PD | No | SD |
| PD | Any | Yes or No | PD |
| Any | PD | Yes or No | PD |
| Any | Any | Yes | PD |

Patients with a global deterioration of health status requiring discontinuation of treatment without objective evidence of disease progression at that time should be classified as having “symptomatic deterioration.” Every effort should be made to document the objective progression even after discontinuation of treatment.

In some circumstances it may be difficult to distinguish residual disease from normal tissue. When the evaluation of complete response depends on this determination, it is recommended that the residual lesion be investigated (fine needle aspirate/biopsy) to confirm the complete response status.

Confirmation

The main goal of confirmation of objective response is to avoid overestimating the response rate observed. In cases where confirmation of response is not feasible, it should be made clear when reporting the outcome of such studies that the responses are not confirmed.

To be assigned a status of PR or CR, changes in tumor measurements must be confirmed by repeat assessments that should be performed no less than 4 weeks after the criteria for response are first met. Longer intervals as determined by the study protocol may also be appropriate.

In the case of SD, follow-up measurements must have met the SD criteria at least once after study entry at a minimum interval (in general, not less than 6-8 weeks) that is defined in the study protocol

Duration of Overall Response

The duration of overall response is measured from the time measurement criteria are met for CR or PR (whichever status is recorded first) until the first date that recurrence or PD is objectively documented, taking as reference for PD the smallest measurements recorded since the treatment started.

Duration of Stable Disease

SD is measured from the start of the treatment until the criteria for disease progression are met, taking as reference the smallest measurements recorded since the treatment started.

The clinical relevance of the duration of SD varies for different tumor types and grades. Therefore, it is highly recommended that the protocol specify the minimal time interval required between two measurements for determination of SD. This time interval should take into account the expected clinical benefit that such a status may bring to the population under study.

Response Review

For trials where the response rate is the primary endpoint it is strongly recommended that all responses be reviewed by an expert(s) independent of the study at the study's completion. Simultaneous review of the patients' files and radiological images is the best approach.

Reporting of Results

All patients included in the study must be assessed for response to treatment, even if there are major protocol treatment deviations or if they are ineligible. Each patient will be assigned one of the following categories: 1) complete response, 2) partial response, 3) stable disease, 4) progressive disease, 5) early death from malignant disease, 6) early death from toxicity, 7) early death because of other cause, or 9) unknown (not assessable, insufficient data).

All of the patients who met the eligibility criteria should be included in the main analysis of the response rate. Patients in response categories 4-9 should be considered as failing to respond to treatment (disease progression). Thus, an incorrect treatment schedule or drug administration does not result in exclusion from the analysis of the response rate. Precise definitions for categories 4-9 will be protocol specific.

All Conclusions should be Based on all Eligible Patients.

Subanalyses may then be performed on the basis of a subset of patients, excluding those for whom major protocol deviations have been identified (e.g., early death due to other reasons, early discontinuation of treatment, major protocol violations, etc.). However, these subanalyses may not serve as the basis for drawing conclusions concerning treatment efficacy, and the reasons for excluding patients from the analysis should be clearly reported.

The 95% confidence intervals should be provided.

### Example 5

### Treatment of Advanced, Persistent or Recurrent Uterine Carcinosarcoma with a Combination of Paclitaxel, Carboplatin and BA

Patients have advanced (stage III or IV), persistent or recurrent uterine carcinosarcoma with documented disease progression. Histologic confirmation of the original primary tumor is required.

All patients will have measurable disease. Measurable disease is defined as at least one lesion that can be accurately measured in at least one dimension (longest dimension to be recorded). Each lesion must be ≧20 mm when measured by conventional techniques, including palpation, plain x-ray, CT, and MRI, or ≧10 mm when measured by spiral CT.

Patients will have at least one “target lesion” to be used to assess response on this protocol as defined by RECIST (Section 8.1). Tumors within a previously irradiated field will be designated as “non-target” lesions unless progression is documented or a biopsy is obtained to confirm persistence at least 90 days following completion of radiation therapy. In addition, patients must have recovered from effects of recent surgery, radiotherapy or other therapy, and should be free of active infection requiring antibiotics.

Any hormonal therapy directed at the malignant tumor must be discontinued at least one week prior to registration. Continuation of hormone replacement therapy is permitted.

Patients must have adequate:
Bone marrow function: Platelet count greater than or equal to 100,000/microliter, and ANC count greater than or equal to 1,500/microliter, equivalent to CTCAE v3.0 grade 1.
Renal function: creatinine less than or equal to 1.5× institutional upper limit normal (ULN), CTCAE v3.0 grade 1.
Hepatic function: Bilirubin less than or equal to 1.5×ULN(CTCAE v3.0 grade 1). SGOT and alkaline phosphatase less than or equal to 2.5×ULN(CTCAE v3.0 grade 1).
Neurologic function: Neuropathy (sensory and motor) less than or equal to CTCAE v3.0 grade 1.
Patients of childbearing potential must have a negative serum pregnancy test prior to the study entry and be practicing an effective form of contraception.

Ineligible Patients:

Patients who have received prior cytotoxic chemotherapy for management of uterine carcinosarcoma.

Patients with a history of other invasive malignancies, with the exception of non-melanoma skin cancer and other specific malignancies as noted in Sections 3.23 and 3.24 are excluded if there is any evidence of other malignancy being present within the last five years. Patients are also excluded if their previous cancer treatment contraindicates this protocol therapy.

Patients who have received prior radiotherapy to any portion of the abdominal cavity or pelvis OTHER THAN for the treatment of uterine carcinosarcoma within the last five years are excluded. Prior radiation for localized cancer of the breast, head and neck, or skin is permitted, provided that it is completed more than three years prior to registration, and the patient remains free of recurrent or metastatic disease.

Patients MAY have received prior adjuvant chemotherapy for localized uterine cancer, provided that it is completed more than three years prior to registration, and that the patient remains free of recurrent or metastatic disease.

Symptomatic or untreated brain metastases requiring concurrent treatment, inclusive of but not limited to surgery, radiation, and corticosteroids.

Myocardial infarction (MI) within 6 months of study day 1, unstable angina, congestive heart failure (CHF) with New York Heart Association (NYHA)>class II, or uncontrolled hypertension.

History of seizure disorder or currently on anti-seizure medication.

### Study Modalities

Carboplatin (Paraplatin®, NSC #241240)

Formulation: Carboplatin is supplied as a sterile lyophilized powder available in single-dose vials containing 50 mg, 150 mg and 450 mg of carboplatin for administration by intravenous infusion. Each vial contains equal parts by weight of carboplatin and mannitol.

Solution Preparation: Immediately before use, the content of each vial must be reconstituted with either sterile water for injection, USP, 5% dextrose in water, or 0.9% sodium chloride injection, USP, according to the following schedule:

| Vial Strength | Diluent Volume |
|---|---|
| 50 mg | 5 ml |
| 150 mg | 15 ml |
| 450 mg | 45 ml |

These dilutions all produce a carboplatin concentration of 10 mg/ml.

NOTE: Aluminum reacts with carboplatin causing precipitate formation and loss of potency. Therefore, needles or intravenous sets containing aluminum parts that may come in contact with the drug must not be used for the preparation or administration of carboplatin.

Storage: Unopened vials of carboplatin are stable for the life indicated on the package when stored at controlled room temperature and protected from light.

Stability: When prepared as directed, carboplatin solutions are stable for eight hours at room temperature. Since no antibacterial preservative is contained in the formulation, it is recommended that carboplatin solutions be discarded eight hours after dilution.

Supplier: Commercially available from Bristol-Myers Squibb Company.

Paclitaxel (Taxol®, NSC #673089)

Formulation: Paclitaxel is a poorly soluble plant product from Taxus baccata. Improved solubility requires a mixed solvent system with further dilutions of either 0.9% sodium chloride or 5% dextrose in water.

Paclitaxel is supplied as a sterile solution concentrate, 6 mg/ml in 5 ml vials (30 mg/vial) in polyoxyethylated castor oil (Cremophor EL) 50% and dehydrated alcohol, USP, 50%. The contents of the vial must be diluted just prior to clinical use. It is also available in 100 and 300 mg vials.

Solution Preparation: Paclitaxel, at the appropriate dose, will be diluted in 500-1000 ml of 0.9% Sodium Chloride injection, USP or 5% Dextrose injection, USP (D5W) (500 ml is adequate if paclitaxel is a single agent). Paclitaxel must be prepared in glass or polyolefin containers due to leaching of diethylhexlphthalate (DEHP) plasticizer from polyvinyl chloride (PVC) bags and intravenous tubing by the Cremophor vehicle in which paclitaxel is solubilized.

NOTE: Formation of a small number of fibers in solution (within acceptable limits established by the USP Particulate Matter Test for LVPs) has been observed after preparation of paclitaxel. Therefore, in-line filtration is necessary for administration of paclitaxel solutions. In-line filtration should be accomplished by incorporating a hydrophilic, microporous filter of pore size not greater than 0.22 microns (e.g.: IVEX-II, IVEX-HP or equivalent) into the IV fluid pathway distal to the infusion pump. Although particulate formation does not indicate loss of drug potency, solutions exhibiting excessive particulate matter formation should not be used.

Storage: The intact vials can be stored in a temperature range between 20-25° C. (36-77° F.) in the original package. Freezing or refrigeration will not adversely affect the stability of the product.

Stability: All solutions of paclitaxel exhibit a slight haziness directly proportional to the concentration of drug and the time elapsed after preparation, although when prepared as described above, solutions of paclitaxel (0.3-1.2 mg/mL) are physically and chemically stable for 27 hours at ambient temperature (approximately 25° C.) and room lighting conditions.

Supplier: Commercially available from Bristol-Myers Squibb Company.

Administration: Paclitaxel, at the appropriate dose and dilution, will be given as a 3-hour continuous IV infusion. Paclitaxel will be administered via an infusion control device (pump) using non-PVC tubing and connectors, such as the IV administration sets (polyethylene or polyolefin) that are used to infuse parenteral Nitroglycerin. Nothing else is to be infused through the line where paclitaxel is being administered. See section 5.2.

BA (4-Iodo-3-Nitrobenzamide)

BA will be manufactured and packaged on behalf of BiPar Sciences and distributed using BiPar-approved clinical study drug distribution procedures. BA will be presented as a liquid sterile product in 10 mL single-entry vials. BA is formulated in 25% hydroxypropylbetacyclodextrin/10 mM phosphate buffer, pH 7.4 with an active ingredient concentration of 10 mg/mL. Each vial contains not less than 9.0 mL of extractable volume. Information presented on the labels for the study drug will comply with ICH requirements and those of the US Food and Drug Administration (FDA). Bulk vials of BA will be shipped in cartons of 10 vials per carton and will be labeled with a one-part label. The label will contain the following information: The U.S. cautionary statement for investigational drugs, study number, product name, concentration, storage, retest date, and the name of the study sponsor.

Solution Preparation: BA will be prepared as described below and administered intravenously over a one-hour period:

Calculate the amount (4 mg/kg) of BA required for dosing by using the subject's baseline weight multiplied by the dose level. For example

Subject baseline weight=70 kg

Dose=4 mg/kg

Required dose=(4 mg/kg×70 kg)=280 mg BA

Divide the dose of BA needed by the BA concentration in the vial (10 mg/mL) to determine the quantity in mL of BA drug product required for administration. Example:

280 mg÷10 mg/mL=28 mL

Calculate the number of vials of BA at 10 mL per vial to obtain the required volume. (Using this example, 3 vials would be needed.) An additional vial may be used if needed to obtain the needed volume of BA.

Withdraw by syringe the appropriate volume of BA drug product from the vial and set it aside while preparing the IV bag as follows:

It is recommended that a total of 250 mL of solution be in the IV bag and delivered over a one hour period. Use an IV solution of either 0.9% NS or D5W. If starting with an IV bag containing greater than 250 mL of solution, remove and discard the excess solution plus the total volume of drug product to be added to the solution. Inject the calculated volume of BA drug product into the IV bag and ensure adequate mixing. Attach the IV tubing and prime it with the solution. Note: It is acceptable to use an empty IV bag and inject the BA volume as calculated, and then add the 0.9% NS or 5DW to reach a total volume of 250 mL. This would likely be useful for BA volumes of greater than 50 mL.

Storage: The BA drug product vials must be stored at 2-8° C. and protected from light. Keep the drug product vials in the original carton and place in a 2-8° C. temperature-controlled unit. BA may be stored at 25° C. for as long as 24 hours as needed. If BA is determined to have not been handled under these storage conditions, please contact BiPar immediately. Do not use vials that have not been stored at the recommended storage conditions without authorization from BiPar.

Stability: Administer BA within 8 hours after preparation. The dosing solution should be kept at ambient (room) temperature until administered to a study subject.

Supplier: BiPar Sciences Inc.

Treatment Plan

Paclitaxel 175 mg/m² as a three-hour infusion followed by Carboplatin dosed to an AUC=6.0 over 30 minutes, on Day 1, every 21 days plus BA 4 mg/kg IV over a one hour infusion period twice weekly beginning on Day 1 (doses of BA must be separated by at least 2 days) until disease progression or adverse affects limit further therapy. This three-week period of time is considered one treatment cycle. Number of cycles beyond complete clinical response will be at the discretion of the treating physician. Patients not meeting the criteria for progression of disease (partial response or stable disease) should be continued on study treatment until limited by toxicity.

Dosing of Carboplatin: The dose will be calculated to reach a target area under the curve (AUC) of concentration×time according to the Calvert formula using an estimated glomerular filtration rate (GFR) from the Jelliffe formula. The initial dose will be AUC=6 infused over 30 minutes.

The initial dose of carboplatin must be calculated using GFR. In the absence of new renal obstruction or other renal toxicity greater than or equal to CTCAE v3.0 grade 2 (serum creatinine>1.5×ULN), the dose of carboplatin will not be recalculated for subsequent cycles, but will be subject to dose modification as noted.

In patients with an abnormally low serum creatinine (less than or equal to 0.6 mg/dl), due to reduced protein intake and/or low muscle mass, the creatinine clearance should be estimated using a minimum value of 0.6 mg/dl. If a more appropriate baseline creatinine value is available within 4 weeks of treatment that may also be used for the initial estimation of GFR.

Calvert Formula Carboplatin dose (mg)=target AUC×(GFR+25).

For the purposes of this protocol, the GFR is considered to be equivalent to the creatinine clearance. The creatinine clearance (Ccr) is estimated by the method of Jelliffe using the following formula: {98−[0.8 (age−20)]} Ccr=0.9×Scr Where: Ccr=estimated creatinine clearance in ml/min; Age=patient's age in years (from 20-80); Scr=serum creatinine in mg/dl. In the absence of new renal obstruction or elevation of serum creatinine above 1.5×ULN (CTCAE v3.0 grade 2), the dose of carboplatin will not be recalculated for subsequent cycles, but will be subject to dose modification for hematologic criteria and other events as noted.

Suggested Method of Chemotherapy Administration: The regimen can be administered in an outpatient setting. Paclitaxel will be administered in a 3-hour infusion followed by carboplatin over 30 minutes, followed by BA over one hour. BA will be administered intravenously (as an infusion over a time period of one hour) twice weekly for the duration of the study. Doses of BA must be separated by at least 2 days (for example doses can be given on Monday/Thursday, Monday/Friday, or Tuesday/Friday). An antiemetic regimen is recommended for day 1 treatment with paclitaxel and carboplatin treatment. The antiemetic regimen used should be based on peer-reviewed consensus guidelines. Prophylactic antiemetics are not needed for BA doses given alone.

Preparative Regimen for Paclitaxel: Paclitaxel will be administered as a 3-hour infusion on this study. For all cycles where paclitaxel is to be administered, it is recommended that a preparative regimen be employed to reduce the risk associated with hypersensitivity reactions. This regimen should include dexamethasone (either IV or PO), anti-histamine H1 (such as diphenhydramine) and anti-histamine H2 (such as cimetidine, ranitidine, or famotidine.)

Maximum body surface area used for dose calculations will be 2.0 m².

If side effects are not severe, a patient may remain on a study agent indefinitely at the investigator's discretion. Patients achieving a complete clinical response may be continued for additional cycles at the discretion of the treating physician.

Evaluation Criteria

Parameters of Response—RECIST Criteria

Measurable disease is defined as at least one lesion that can be accurately measured in at least one dimension (longest dimension to be recorded). Each lesion must be ≧20 mm when measured by conventional techniques, including palpation, plain x-ray, CT, and MRI, or ≧10 mm when measured by spiral CT.

Baseline documentation of “Target” and “Non-Target” lesions

All measurable lesions up to a maximum of 5 lesions per organ and 10 lesions in total representative of all involved organs should be identified as target lesions and will be recorded and measured at baseline. Target lesions should be selected on the basis of their size (lesions with the longest dimension) and their suitability for accurate repetitive measurements by one consistent method of assessment (either by imaging techniques or clinically). A sum of the longest dimension (LD) for all target lesions will be calculated and reported as the baseline sum LD. The baseline sum LD will be used as reference to further characterize the objective tumor response of the measurable dimension of the disease.

All other lesions (or sites of disease) should be identified as non-target lesions and should also be recorded at baseline. Measurements are not required and these lesions should be followed as “present” or “absent”.

All baseline evaluations of disease status should be performed as close as possible to the start of treatment and never more than 4 weeks before the beginning of treatment.

Best Response

Measurement of the longest dimension of each lesion size is required for follow up. Change in the sum of these dimensions affords some estimate of change in tumor size and hence therapeutic efficacy. All disease must be assessed using the same technique as baseline. Reporting of these changes in an individual case should be in terms of the best response achieved by that case since entering the study.

Complete Response (CR) is disappearance of all target and non-target lesions and no evidence of new lesions documented by two disease assessments at least 4 weeks apart.

Partial Response (PR) is at least a 30% decrease in the sum of longest dimensions (LD) of all target measurable lesions taking as reference the baseline sum of LD. There can be no unequivocal progression of non-target lesions and no new lesions. Documentation by two disease assessments at least 4 weeks apart is required. In the case where the ONLY target lesion is a solitary pelvic mass measured by physical exam, which is not radiographically measurable, a 50% decrease in the LD is required.

Increasing Disease is at least a 20% increase in the sum of LD of target lesions taking as references the smallest sum LD or the appearance of new lesions within 8 weeks of study entry. Unequivocal progression of existing non-target lesions, other than pleural effusions without cytological proof of neoplastic origin, in the opinion of the treating physician within 8 weeks of study entry is also considered increasing disease (in this circumstance an explanation must be provided). In the case where the ONLY target lesion is a solitary pelvic mass measured by physical exam, which is not radiographically measurable, a 50% increase in the LD is required.

Symptomatic deterioration is defined as a global deterioration in health status attributable to the disease requiring a change in therapy without objective evidence of progression.

Stable Disease is any condition not meeting the above criteria.

Inevaluable for response is defined as having no repeat tumor assessments following initiation of study therapy for reasons unrelated to symptoms or signs of disease.

Progression (measurable disease studies) is defined as ANY of the following:

At least a 20% increase in the sum of LD target lesions taking as reference the smallest sum LD recorded since study entry

In the case where the ONLY target lesion is a solitary pelvic mass measured by physical exam which is not radiographically measurable, a 50% increase in the LD is required taking as reference the smallest LD recorded since study entry

The appearance of one or more new lesions

Death due to disease without prior objective documentation of progression

Global deterioration in health status attributable to the disease requiring a change in therapy without objective evidence of progression

Unequivocal progression of existing non-target lesions, other than pleural effusions without cytological proof of neoplastic origin, in the opinion of the treating physician (in this circumstance an explanation must be provided)

Recurrence (non-measurable disease studies) is defined as increasing clinical, radiological or histological evidence of disease since study entry.

Survival is the observed length of life from entry into the study to death or the date of last contact.

Progression-Free Survival (measurable disease studies) is the period from study entry until disease progression, death or date of last contact.

Recurrence-Free Survival (non-measurable disease studies) is the period from study entry until disease recurrence, death or date of last contact.

Subjective Parameters including performance status, specific symptoms, and side effects are graded according to the CTCAE v3.0.

Duration of Study

Patients will receive therapy until disease progression or intolerable toxicity intervenes. The patient can refuse the study treatment at any time. Patients with compete clinical response to therapy will be continued on therapy with additional numbers of cycles at the treating physician's discretion. Patients with partial response or stable disease should be continued on therapy unless intolerable toxicity prohibits further therapy.

All patients will be treated (with completion of all required case report forms) until disease progression or study withdrawal. Patients will then be followed (with physical exams and histories) every three months for the first two years and then every six months for the next three years. Patients will be monitored for delayed toxicity and survival for this 5-year period with Q forms submitted to the GOG Statistical and Data Center, unless consent is withdrawn.

### Example 6

### A Phase 2, Single Arm Study of 4-iodo-3-nitrobenzamide in Patients with BRCA-1 or BRCA-2 Associated Advanced Epithelial Ovarian, Fallopian Tube, or Primary Peritoneal Cancer

This is a single institution, single arm study of 4-iodo-3-nitrobenzamide (BA) in patients with advanced BRCA-1 or BRCA-2 associated epithelial ovarian, fallopian tube, or primary peritoneal cancer. The goal of this study is to determine if BA is efficacious in this patient population. Eligible patients will have received initial treatment with platinum/taxane combination therapy and have no curative options as determined by their physician. There will be no limit on the number of prior therapies. A maximum of 35 patients will be treated in this study using a Simon two-stage optimal design.

The protocol schema is shown below. Patients will be treated with the investigational agent, BA, intravenously twice weekly on days 1 and 4 for a total of 8 weeks. This will comprise one cycle of therapy. Baseline CT or MRI scans and CA125 levels will occur within the 4 weeks prior to cycle 1 day 1. Reassessment of disease will occur in the eighth week of cycle one. Patients will continue with additional cycles of treatment as long as they have stable or responding disease (per RECIST criteria) and wish to remain on study.

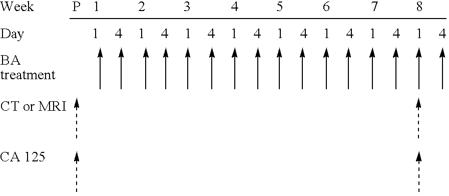

Additional exploratory components to this study include assessment of historical paraffin-embedded tumor tissue for PARP-1 gene expression, evaluation of peripheral blood mononuclear cells (PBMCs) for PARP inhibition, sequencing of BRCA1 or BRCA2 for secondary intragenic mutations, and collection of ascites fluid as appropriate for biomarker analyses.

### Objectives and Scientific Aims

### Primary

To evaluate the response rate (per RECIST) to BA when administered at 8 mg/kg intravenously twice weekly in subjects with BRCA-1 or BRCA-2 associated advanced epithelial ovarian, fallopian tube, or primary peritoneal cancer.

### Secondary

To evaluate the clinical benefit rate (overall response rate and stable disease) of BA when administered at 8 mg/kg intravenously twice weekly in subjects with BRCA-1 or BRCA-2 associated advanced epithelial ovarian, fallopian tube, or primary peritoneal cancer.

To evaluate progression free survival (PFS) and overall survival (OS) in subjects receiving BA.

To evaluate response as measured by CA-125 level in subjects receiving BA.

To evaluate the safety and tolerability of BA when administered at 8 mg/kg intravenously twice weekly.

### Exploratory

To assess the extent of PARP inhibition in peripheral blood mononuclear cells (PBMCs).
To assess PARP-1 gene expression in tumor samples and correlate expression levels to response to BA.
To identify secondary intragenic mutations and correlate with response to BA.
To collect ascites fluid from patients when it is clinically necessary for tumor banking.

### Rationale for the Study

The goal of the present study is to determine the efficacy of BA in patients with BRCA-associated ovarian cancer. Given the unique susceptibility of BRCA deficient tumor cells to PARP inhibition, treatment with BA may offer this subset of ovarian cancer patients an effective therapy with less toxicity when compared to currently available regimens. Response rates to currently available chemotherapeutics in patients with a less than 12 month disease-free interval range from 15-20%.²³ A phase I study using a different PARP inhibitor showed responses in 5/11 BRCA-associated ovarian cancer patients.¹⁹ Thus, this study is poared to see a difference between a 10 and 30% response rate.

### Design

This is a single arm study of BA in patients with BRCA-1 or BRCA-2 associated advanced epithelial ovarian, fallopian tube, or primary peritoneal cancer. Patients will be enrolled using a Simon optimal two-stage statistical design (Simon, Controlled Clin Trials, 10:1-10, 1989). A total of 35 patients will be enrolled in this study. Twelve will be enrolled in the first stage. If 2/12 patients in the first stage respond (as defined by RECIST criteria) to treatment, 23 additional patients will be enrolled in the second stage. If at least 6/35 patients respond at the end of the trial, then this study will be declared positive. This study will be poared to see a difference between a 10% and 30% response rate with a type 1 error=0.10 and a type 2 error=0.10. Secondary endpoints will be tabulated and reported descriptively. Exploratory studies will be hypothesis-generating for future studies and will be reported descriptively.

### Criteria for Subject Eligibility

### Subject Inclusion Criteria

Female, age 18 or older.
Histologically or cytologically confirmed advanced epithelial ovarian cancer, fallopian tube cancer or primary peritoneal cancer (stage III or IV).
Patients must have received at least one regimen of platinum/taxane therapy.
Confirmed BRCA1 or BRCA2 status.
One or more measurable lesions, at least 10 mm in longest diameter by spiral CT scan or 20 mm in longest diameter when measured with conventional techniques (palpation, plain x-ray, CT or MRI).
Karnofsky performance status≧70%.
Estimated life expectancy of at least 16 weeks.

### Subject Exclusion Criteria

Screening clinical laboratory values:
   Absolute neutrophil count<1500/□L
   Platelet count<100,000/μL
   Hemoglobin<8.5 g/dL
   Serum bilirubin>2.0× upper limit of normal (ULN)
   AST and ALT>2.5×ULN (AST and ALT>5×ULN for subjects with liver metastases)
   Serum creatinine>1.5×ULN
Any anti-cancer therapy within 21 days prior to day 1.
Any other malignancy within 3 years of day 1, except adequately treated carcinoma in situ of the cervix, ductal carcinoma in situ (DCIS) of the breast, or basal or squamous cell skin cancer.
Active viral infection including HIV/AIDS, Hepatitis B or Hepatitis C infection.
Active central nervous system or brain metastases.
History of seizures or current treatment with anti-epileptic medication.
Persistent grade 2 or greater toxicities from prior therapy, excluding alopecia.

### Treatment/Intervention Plan

This phase II, single-arm, single institution study will accrue a maximum of 35 patients with advanced epithelial ovarian, fallopian tube, or primary peritoneal cancer. The estimated rate of accrual is 2-4 patients per month.

All treatments will be given in the outpatient setting. Patients who qualify for enrollment on the study after the pre-treatment screening assessment described above will initiate treatment. BA at a dose of 8 mg/kg will be given intravenously twice weekly for a total of eight weeks. Treatment will be administered on days 1 and 4 of each week. BA doses must be separated by 2 treatment-free days. Patients will have radiographic assessment of their disease during week eight of therapy. Patients without disease progression (SD, PR, or CR) may continue on therapy for additional cycles.

### Routine Monitoring During Treatment

During cycle 1, patients will have their vital signs measured weekly. They will be evaluated every two weeks (days 1, 15, 29, 43) with a complete history and physical exam, performance status assessment, weight, complete blood count, coagulation studies (PT/PTT), comprehensive metabolic panel, and magnesium level. Patients will be instructed to report any changes in concomitant medications or side effects as they occur while on study. Radiographic imaging using CT or MRI, EKG, and a blood CA-125 level will be done during the eighth week of each cycle.

### Experimental Procedures During Treatment

Blood samples (5 ml) will be collected 1 hour pre-, immediately pre- and immediately post-BA dose on days 1 and 15 of cycles 1 and 2 to determine the level of PARP inhibition in peripheral blood mononuclear cells. A blood sample (10 ml) will be collected once for germline DNA extraction. This will be used for the correlative studies assessing secondary mutations of BRCA1 or 2. This may occur within 14 days of starting treatment or pre-treatment on day 1 of cycle 1. In patients undergoing clinically indicated paracenteses while on treatment, a sample will be collected for tumor banking. This may occur once for each patient at any time while on treatment.

Patients will have a final follow-up visit once they have been withdrawn from the study for any reason. This visit will occur at least four weeks after the last dose of BA. The following assessments will occur at this visit:
Clinical evaluation including medical history, physical examination, Karnofsky performance status, height, weight, vital signs (blood pressure, respiration rate, pulse, temperature)
Recording of concomitant medications
Blood sampling for:
   CA-125
   Complete blood count (CBC)
   Coagulation studies including prothrombin time (PT) and partial thromboplastin time (PTT)
   Comprehensive metabolic panel (BUN, creatinine, Na, Cl, CO2, Ca, Glucose, Total bilirubin, Total protein, albumin. Alkaline phosphatase, AST, ALT)
   Magnesium
Toxicity assessment

Patients who have stable disease at the time of study withdrawal will be encouraged to continue to have radiographic assessment of their disease burden with a CT or MRI scan and a CA-125 level at least every 3 months after they have stopped taking BA. This will be used for determining the secondary endpoint of PFS. Study staff will continue to contact patients every 3 months for the first year and every 6 months following the first year to assess disease status and survival.

### Treatment Modifications

### Dose Reductions

To date, no serious adverse events or grade 3 or 4 toxicities have been associated with BA. The drug appears to be safe and well-tolerated. However, if a patient experiences any grade 3 or 4 toxicity, drug should be held until the toxicity resolves to <grade 2.

### Scheduling Delays and Missed Doses

If scheduling constraints arise such that the patient is unable to be treated on day 1 or 4 of a given week, shifts of the schedule by one day are permitted as outlined below. Treatment days are indicated by underlined bold font.

| | |
|---|---|
| Standard treatment schedule | 1 2 3 4 5 6 7 1 2 3 4 5 6 7 1 . . . |
| Modified allowed schedule if | 1 2 3 4 5 6 7 1 2 3 4 5 6 7 1 . . . |
| day 4 is missed | |
| Modified allowed schedule if | 1 2 3 4 5 6 7 1 2 3 4 5 6 7 1 . . . |
| day 1 is missed | |

Since there must be a mandatory 2-day treatment-free interval between doses, if a patient is unable to be treated the day following the missed dose as shown above, the dose will be skipped. The patient would then resume treatment on the next scheduled day 1 or 4. Two skipped doses will be allowed while on study. Patients who skip 3 doses due to scheduling conflicts will be removed from the study protocol.

### Exploratory Studies/Correlative Science

### PARP Inhibition in Peripheral Blood Mononuclear Cells (PBMCs)

The functional activity of PARP 1 hour before, immediately before and after treatment of BA will be determined using a PARP activity assay in peripheral blood mononuclear cells (PBMCs). This will be done on days 1 and 15 of cycles 1 and 2. PBMCs will be prepared from 5 mL blood samples according to procedures described in detail in the study manual and PARP activity/inhibition will be measured. Each blood sample will be analyzed in triplicate and the PARP activity will be reported as relative light units (RLU), normalized to a standard curve. The sample one hour prior to BA will be compared to the sample immediately prior to BA dose to evaluate whether there is normal variability in PARP activity at differing times in the day despite pharmacologic intervention.

### PARP-1 Gene Expression in Tumor Samples

PARP gene expression will be evaluated in patients' tumor specimens using multiplex RT-PCR. Prior to initiating therapy, a paraffin block or 6 slides from a paraffin-embedded tumor specimen will be collected for each patient. A paraffin block or 4 slides from paraffin-embedded normal tissue will also be collected. The slides should contain ≧75% of tumor or normal tissue, respectively. The normal specimen does not have to be of the same tissue type as the tumor (i.e. normal fallopian tube, uterine tissue, or other normal tissue specimen from initial surgery could be used) and will be used as a control specimen for PARP RT-PCR. The tumor sample may be from the patient's original surgery or other tumor biopsy specimens. Preferably, the specimen will be from the most recent tumor sampling procedure in the event that PARP expression has changed over time. Two of the six tumor slides will be used for correlative immunohistochemistry analysis.

### Secondary Intragenic Mutation Analysis

In this study, we will collect germline DNA from each patient from 10 ml of peripheral blood. The blood sample will be collected in one or two purple top blood collection tubes with EDTA. The specimens will be transported to the Gynecology Research Lab where DNA extraction and dilution will occur. Tumor tissue will be obtained from paraffin blocks or four unstained slides. Tissue will be trimmed to obtain at least 80% tumor cell nuclei in the final specimen. Tumor DNA will be extracted according to standard laboratory methods. The tumor DNA will be sequenced for the entire coding region of either BRCA1 or BRCA2 based on whichever mutation the patient is know to carry. Sequencing will be performed through the HOPP translational core. Semi-automated sequence interpretation will be performed to identify any secondary mutations or deletions. All identified variants will be confirmed by a second PCR amplification and sequencing. Germline DNA will be sequenced for positive cases to confirm the somatic nature of the mutation or deletion.

### Ascites Fluid Tumor Banking

Patients with ascites who need palliative or therapeutic paracenteses during the study will have ascites fluid collected for tumor banking. Future use of these samples will require IRB approval as per MSKCC guidelines. Ascites fluid tumor banking will be an invaluable source of ovarian tumor cells for biomarker analysis.

### Criteria for Therapeutic Response/Outcome Assessment

The primary objective of the study is to determine the response rate in subjects treated with BA. Response will be determined using RECIST criteria. The parameters required for the initial assessment of measurable disease and response are as follows:

### Baseline Measurable Disease—GOG RECIST Criteria

Measurable disease is defined as at least one lesion that can be accurately measured in at least one dimension (longest dimension to be recorded). Each lesion must be ≧20 mm when measured by conventional techniques, including palpation, plain x-ray, CT, and MRI, or ≧10 mm when measured by spiral CT.

### Baseline Documentation of “Target” and “Non-Target” Lesions

All measurable lesions up to a maximum of 5 lesions per organ and 10 lesions in total representative of all involved organs should be identified as target lesions and will be recorded and measured at baseline. Target lesions should be selected on the basis of their size (lesions with the longest dimension) and their suitability for accurate repetitive measurements by one consistent method of assessment (either by imaging techniques or clinically). A sum of the longest dimension (LD) for all target lesions will be calculated and reported as the baseline sum LD. The baseline sum LD will be used as reference to further characterize the objective tumor response of the measurable dimension of the disease.

All other lesions (or sites of disease) should be identified as non-target lesions and should also be recorded at baseline. Measurements are not required and these lesions should be followed as “present” or “absent”.

All baseline evaluations of disease status should be performed as close as possible to the start of treatment and never more than 4 weeks before the beginning of treatment.

Best Response

Measurement of the longest dimension of each lesion size is required for follow-up. Change in the sum of these dimensions affords some estimate of change in tumor size and hence therapeutic efficacy. All disease must be assessed using the same technique as baseline. Reporting of these changes in an individual case should be in terms of the best response achieved by that case since entering the study.

Complete Response (CR) is disappearance of all target and non-target lesions and no evidence of new lesions. A confirmed complete response requires documentation by two disease assessments at least 4 weeks apart.

Partial Response (PR) is at least a 30% decrease in the sum of longest dimensions (LD) of all target measurable lesions taking as reference the baseline sum of LD. There can be no unequivocal progression of non-target lesions and no new lesions. A confirmed partial response requires documentation by two disease assessments at least 4 weeks apart. In the case where the ONLY target lesion is a solitary pelvic mass measured by physical exam, which is not radiographically measurable, a 50% decrease in the LD is required.

Increasing Disease is at least a 20% increase in the sum of LD of target lesions taking as references the smallest sum LD or the appearance of new lesions within 8 weeks of study entry. Unequivocal progression of existing non-target lesions, other than pleural effusions without cytological proof of neoplastic origin, in the opinion of the treating physician within 8 weeks of study entry is also considered increasing disease (in this circumstance an explanation must be provided). In the case where the ONLY target lesion is a solitary pelvic mass measured by physical exam, which is not radiographically measurable, a 50% increase in the LD is required.

Symptomatic deterioration is defined as a global deterioration in health status attributable to the disease requiring a change in therapy without objective evidence of progression.

Stable Disease is any condition not meeting the above criteria.

Inevaluable for response is defined as having no repeat tumor assessments following initiation of study therapy for reasons unrelated to symptoms or signs of disease.

Progression (measurable disease studies) is defined as ANY of the following:

At least a 20% increase in the sum of LD target lesions taking as reference the smallest sum LD recorded since study entry

In the case where the ONLY target lesion is a solitary pelvic mass measured by physical exam which is not radiographically measurable, a 50% increase in the LD is required taking as reference the smallest LD recorded since study entry

The appearance of one or more new lesions

Death due to disease without prior objective documentation of progression

Global deterioration in health status attributable to the disease requiring a change in therapy without objective evidence of progression

Unequivocal progression of existing non-target lesions, other than pleural effusions without cytological proof of neoplastic origin, in the opinion of the treating physician (in this circumstance an explanation must be provided)

A summary of how to assess RECIST response is shown below

| | Non-target | New | Overall |
|---|---|---|---|
| Target Lesions | lesions | Lesions | response |
| CR | CR | No | CR |
| CR | SD | No | PR |
| PR | CR or SD | No | PR |
| CR or PR or SD | UNK | No | UNK |
| UNK | CR or SD or | No | UNK |
| | UNK | | |
| SD | CR or SD | No | SD |
| PD | Any | Any | PD |
| Any | PD | Any | PD |
| Any | Any | Yes | PD |

CR = Complete response;
PR = Partial Response;
SD = Stable Disease;
PD = Progressive Disease;
UNK = unknown

Secondary endpoints of the study include evaluating progression free survival and overall survival, safety, and CA125 response. These will be determined using the following parameters:

Progression-Free Survival is the period from study entry until disease progression, death or date of last contact.

Overall Survival is the observed length of life from entry into the study to death or the date of last contact.

Safety Parameters including performance status, specific symptoms, and side effects are graded according to the CTCAE v3.0.

### CA-125 Response Guidelines

Subjects with elevated CA-125 (>50 U/mL) on 2 occasions at least one week apart before initiating study treatment will be evaluated for CA-125 response during the study.

Complete Response (CR): A decrease in CA-125 levels to within the normal range that is confirmed by a repeat assessment no less than 4 weeks later.

Partial Response (PR): A decrease in CA-125 levels by >50% that is confirmed by a repeat assessment no less than 4 weeks later.

Stable disease (SD): Any CA-125 change that does not fit the definition of PD, PR, or CR.

Progressive disease (PD): A doubling of the nadir CA-125 level that is higher than the upper limit of normal that is confirmed by a repeat assessment no less than 4 weeks later.

### Biostatistics

Primary Endpoint

This is a single arm study of BA in patients with BRCA-1 or BRCA-2 associated advanced epithelial ovarian, fallopian tube, or primary peritoneal cancer. The primary endpoint is response rate defined as CR+PR. Patients will be evaluated for response at the end of the first cycle of therapy. Patients will be enrolled using a Simon optimal two-stage statistical design.¹ A total of 35 patients will be enrolled in this study. Twelve will be enrolled in the first stage. If 2/12 patients in the first stage respond (as defined by RECIST criteria) to treatment, 23 additional patients will be enrolled in the second stage. If at least 6/35 patients respond at the end of the trial, then this study will be declared positive. This study will be poared to see a difference between a 10% and 30% response rate with a type 1 error=0.10 and a type 2 error=0.10.

Secondary Endpoints

Clinical benefit rate defined as CR+PR+SD will be reported with a 95% confidence interval. Clinical outcome, such as PFS and OS, will be summarized via median and 95% confidence intervals using the Kaplan Meier method. CA125 response is defined as a decrease to a normal range (0-35) with a confirmatory value followed at the next cycle. CA125 response rate will be reported with a respective 95% confidence interval.

Safety will be described by tabulating toxicities using the NCI Common Terminology Criteria for Adverse Events (version 3.0). Tolerability refers to the ability to adhere to twice weekly dosing without missing more than two doses out of 16 doses as explained in Section 9. Patients with missed doses will be tabulated.

Exploratory Endpoints

Exploratory studies will be hypothesis-generating for future studies and will be reported descriptively. In order to assess the extent of PARP inhibition in PBMCs, an assay measuring PARP enzyme in a continuous scale will be collected before and after treatment at day 1 and day 15 of the first two cycles. The change in PARP enzyme over the four time points will be summarized via median and range and it will be described via graphical summary measures. Appropriate transformations will be used to account for the large variability in PARP RLU scale.

PARP-1 gene expression will be measured in a continuous scale. A non-parametric test will be used to assess whether responders (CR+PR) have a higher expression than non-responders.

The analysis for secondary mutations in BRCA1 or 2 will be reported descriptively. Platinum resistant patients or patients found to be unresponsive to the protocol therapy may have an intragenic deletion that restores the BRCA1/2 open reading frame. The presence of a secondary mutation or deletion will be correlated with the response to protocol therapy. The hypothesis is that patients without a secondary mutation or deletion will respond better than those with a secondary mutation or deletion. A Chi-square test or Fisher's exact test as deemed appropriate will be used to assess whether there is a significant association between secondary mutation or deletion and response to BA treatment. Should evidence prove this hypothesis correct, it may serve as a screening method for future trials involving this drug.

### Example 7

### Effect of BA on Proliferation of Cervical Adenocarcinoma Hela Cells

The effect of BA on the proliferation of cervical adenocarcinoma Hela cells is examined. Cell proliferation is assessed by BrdU assay as described herein.

### Cell Culture

Hela cell is an immortal cell line used in medical research. The cell line was derived from cervical cancer cells. HeLa S3 is a clonal derivative of the parent HeLa line. The HeLa S3 clone has been very useful in the clonal analysis of mammalian cell populations relating to chromosomal variation, cell nutrition, and plaque-forming ability. HeLa cells have been reported to contain human papilloma virus 18 (HPV-18) sequences. Cells are cultured according to the standard protocol (ATCC) in the art. Briefly: 1. Remove and discard culture medium. 2. Briefly rinse the cell layer with 0.25% (w/v) Trypsin-0.53 mM EDTA solution to remove all traces of serum that contains trypsin inhibitor. 3. Add 2.0 to 3.0 ml of Trypsin-EDTA solution to flask and observe cells under an inverted microscope until cell layer is dispersed (usually within 5 to 15 minutes). Cells that are difficult to detach may be placed at 37° C. to facilitate dispersal. 4. Add 6.0 to 8.0 ml of complete growth medium and aspirate cells by gently pipetting. 5. Add appropriate aliquots of the cell suspension to new culture vessels. 6. Incubate cultures at 37° C.

### Materials and Methods

BrdU assay is well known in the art. Briefly, cells are cultured in the presence of the respective test substances in an appropriate 96-well MP at 37° C. for a certain period of time (1 to 5 days, depending on the individual assay system). Subsequently, BrdU is added to the cells and the cells are reincubated (usually 2-24 h). During this labeling period, the pyrimidine analogue BrdU is incorporated in place of thymidine into the DNA of proliferating cells. After removing the culture medium the cells are fixed and the DNA is denatured in one step by adding FixDenat (the denaturation of the DNA is necessary to improve the accessibility of the incorporated BrdU for detection by the antibody). The anti-BrdU-POD antibody is added and the antibody binds to the BrdU incorporated in newly synthesized, cellular DNA. The immune complexes are detected by the subsequent substrate reaction via chemiluminescent detection (based on Cell Proliferation ELISA, BrdU Chemiluminescence Protocol from Roche).

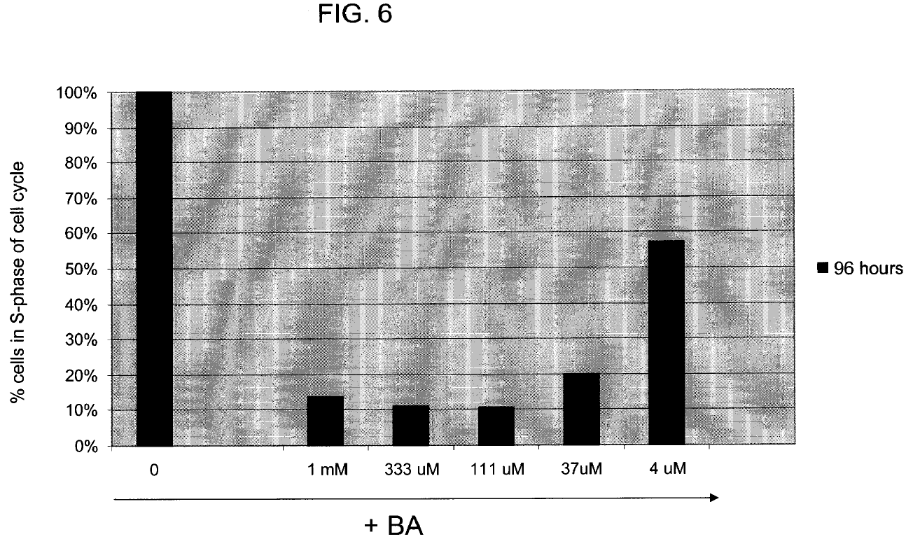

BA is added to the cell culture at various concentrations. As shown in FIG. 6, BA inhibits proliferation of cervical adenocarcinoma Hela cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating uterine cancer or ovarian cancer in a patient, comprising administering to the patient at least one PARP inhibitor.

2. The method of claim 1, wherein at least one therapeutic effect is obtained, said at least one therapeutic effect being reduction in size of a uterine tumor or an ovarian tumor, reduction in metastasis, complete remission, partial remission, pathologic complete response, or stable disease.

3. The method of claim 1, wherein a comparable clinical benefit rate (CBR=CR+PR+SD≧6 months) is obtained with treatment of the PARP inhibitor as compared to treatment with an anti-tumor agent.

4. The method of claim 3, wherein the improvement of clinical benefit rate is at least about 30% over treatment with an anti-tumor agent alone.

5. The method of claim 1, wherein the PARP inhibitor is 4-iodo-3-nitrobenzamide or a metabolite thereof.

6. The method of claim 1, wherein the PARP inhibitor is of Formula (IIa) or a metabolite thereof: 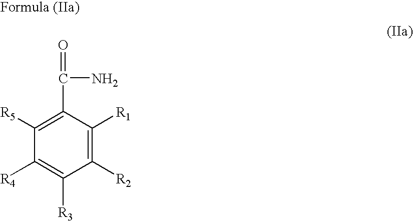
wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

7. The method of claim 1, wherein the uterine cancer is a metastatic uterine cancer.

8. The method of claim 1, wherein the uterine cancer is an endometrial cancer.

9. The method of claim 1, wherein the uterine cancer is recurrent, advanced, or persistent.

10. The method of claim 1, wherein the ovarian cancer is a metastatic ovarian cancer.

11. The method of claim 1, wherein the ovarian cancer is deficient in homologous recombination DNA repair.

12. The method of claim 1, wherein the uterine cancer is deficient in homologous recombination DNA repair.

13. The method of claim 1, wherein the uterine cancer is BRCA deficient.

14. The method of claim 1, wherein the ovarian cancer is BRCA deficient.

15. The method of claim 13 or 14, wherein the BRCA-deficiency is a BRCA1-deficiency, or a BRCA2-deficiency, or both BRCA1 and BRCA2-deficiency.

16. The method of claim 1, wherein the treatment further comprises
(a) establishing a treatment cycle of about 10 to about 30 days in length; and
(b) on from 1 to 10 separate days of the cycle, administering to the patient about 1 mg/kg to about 100 mg/kg of 4-iodo-3-nitrobenzamide, or a molar equivalent of a metabolite thereof.

17. The method of claim 16, wherein the 4-iodo-3-nitrobenzamide or metabolite thereof is administered orally, or as a parenteral injection or infusion, or inhalation.

18. The method of claim 1 further comprises administering to the patient a PARP inhibitor in combination with at least one anti-tumor agent.

19. The method of claim 18, wherein the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof.

20. The method of claim 18, wherein the anti-tumor agent is citabine, capecitabine, valopicitabine or gemcitabine.

21. The method of claim 18, wherein the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871.

22. The method of claim 18 further comprises administering to the patient a PARP inhibitor in combination with more than one anti-tumor agent.

23. The method of claim 18, wherein the anti-tumor agent is administered prior to, concomitant with or subsequent to administering the PARP inhibitor.

24. The method of claim 1 further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

25. A method of treating ovarian cancer or uterine cancer in a patient in need of such treatment, comprising:
(a) obtaining a sample from the patient;
(b) testing the sample to determine whether the patient is BRCA deficient;
(c) if the testing indicates that the patient is BRCA-deficient, treating the patient with at least one PARP inhibitor.

26. The method of claim 25, wherein at least one therapeutic effect is obtained, said at least one therapeutic effect being reduction in size of an ovarian tumor or a uterine tumor, reduction in metastasis, complete remission, partial remission, pathologic complete response, or stable disease.

27. The method of claim 25, wherein a comparable clinical benefit rate (CBR=CR+PR+SD≧6 months) is obtained with treatment of the PARP inhibitor as compared to treatment with an anti-tumor agent.

28. The method of claim 25, wherein the improvement of clinical benefit rate is at least about 30% as compared to treatment with an anti-tumor agent alone.

29. The method of claim 25, wherein the PARP inhibitor is 4-iodo-3-nitrobenzamide or a metabolite thereof.

30. The method of claim 25, wherein the PARP inhibitor is of Formula (IIa) or a metabolite thereof: 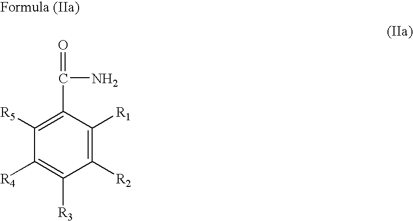
wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

31. The method of claim 25, wherein the sample is a tissue or bodily fluid sample.

32. The method of claim 25, wherein the sample is a tumor sample, a blood sample, a blood plasma sample, a peritoneal fluid sample, an exudate or an effusion.

33. The method of claim 25, wherein the uterine cancer is a metastatic uterine cancer.

34. The method of claim 25, wherein the uterine cancer is an endometrial cancer.

35. The method of claim 25, wherein the uterine cancer is recurrent, advanced, or persistent.

36. The method of claim 25, wherein the ovarian cancer is a metastatic ovarian cancer.

37. The method of claim 25, wherein the ovarian cancer is deficient in homologous recombination DNA repair.

38. The method of claim 25, wherein the uterine cancer is deficient in homologous recombination DNA repair.

39. The method of claim 25, wherein the uterine cancer is BRCA deficient.

40. The method of claim 25, wherein the ovarian cancer is BRCA deficient.

41. The method of claim 39 or 40, wherein the BRCA-deficiency is a BRCA1-deficiency, or a BRCA2-deficiency, or both BRCA1 and BRCA2-deficiency.

42. The method of claim 25, wherein the treatment further comprises
(a) establishing a treatment cycle of about 10 to about 30 days in length; and
(b) on from 1 to 10 separate days of the cycle, administering to the patient about 1 mg/kg to about 100 mg/kg of 4-iodo-3-nitrobenzamide, or a molar equivalent of a metabolite thereof.

43. The method of claim 42, wherein the 4-iodo-3-nitrobenzamide or metabolite thereof is administered orally or as a parenteral injection or infusion, or inhalation.

44. The method of claim 25 further comprises administering to the patient a PARP inhibitor in combination with at least one anti-tumor agent.

45. The method of claim 44, wherein the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof.

46. The method of claim 44, wherein the anti-tumor agent is citabine, capecitabine, valopicitabine or gemcitabine.

47. The method of claim 44, wherein the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871.

48. The method of claim 25 further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

49. A method of treating ovarian cancer or uterine cancer in a patient in need of such treatment, comprising:
(a) obtaining a sample from the patient;
(b) testing the sample to determine a level of PARP expression in the sample;
(c) determining whether the PARP expression exceeds a predetermined level, and if so, administering to the patient at least one PARP inhibitor.

50. The method of claim 49, wherein at least one therapeutic effect is obtained, said at least one therapeutic effect being reduction in size of an ovarian tumor or a uterine tumor, reduction in metastasis, complete remission, partial remission, pathologic complete response, or stable disease.

51. The method of claim 49, wherein a comparable clinical benefit rate (CBR=CR+PR+SD≧6 months) is obtained with treatment of the PARP inhibitor as compared to treatment with an anti-tumor agent.

52. The method of claim 49, wherein the improvement of clinical benefit rate is at least about 30%.

53. The method of claim 49, wherein the PARP inhibitor is 4-iodo-3-nitrobenzamide or a metabolite thereof.

54. The method of claim 49, wherein the PARP inhibitor is of Formula (IIa) or a metabolite thereof: 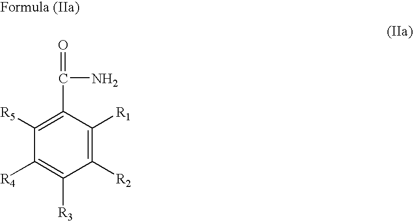
wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

55. The method of claim 49, wherein the sample is a tissue or bodily fluid sample.

56. The method of claim 49, wherein the sample is a tumor sample, a blood sample, a blood plasma sample, a peritoneal fluid sample, an exudate or an effusion.

57. The method of claim 49, wherein the uterine cancer is a metastatic uterine cancer.

58. The method of claim 49, wherein the uterine cancer is an endometrial cancer.

59. The method of claim 49, wherein the uterine cancer is recurrent, advanced, or persistent.

60. The method of claim 49, wherein the ovarian cancer is a metastatic ovarian cancer.

61. The method of claim 49, wherein the ovarian cancer is deficient in homologous recombination DNA repair.

62. The method of claim 49, wherein the uterine cancer is deficient in homologous recombination DNA repair.

63. The method of claim 49, wherein the uterine cancer is BRCA deficient.

64. The method of claim 49, wherein the ovarian cancer is BRCA deficient.

65. The method of claim 63 or 64, wherein the BRCA-deficiency is a BRCA1-deficiency, or a BRCA2-deficiency, or both BRCA1 and BRCA2-deficiency.

66. The method of claim 49, wherein the treatment further comprises
(a) establishing a treatment cycle of about 10 to about 30 days in length; and
(b) on from 1 to 10 separate days of the cycle, administering to the patient about 1 mg/kg to about 100 mg/kg of 4-iodo-3-nitrobenzamide, or a molar equivalent of a metabolite thereof.

67. The method of claim 66, wherein the 4-iodo-3-nitrobenzamide or metabolite thereof is administered orally or as a parenteral injection or infusion, or inhalation.

68. The method of claim 49 further comprises administering to the patient a PARP inhibitor in combination with at least one anti-tumor agent.

69. The method of claim 68, wherein the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof.

70. The method of claim 68, wherein the anti-tumor agent is citabine, capecitabine, valopicitabine or gemcitabine.

71. The method of claim 68, wherein the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871.

72. The method of claim 49 further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

73. A method of treating uterine cancer or ovarian cancer in a patient, comprising administering to the patient a combination of at least one PARP inhibitor and at least one anti-tumor agent.

74. The method of claim 73, wherein at least one therapeutic effect is obtained, said at least one therapeutic effect being reduction in size of a uterine tumor or an ovarian tumor, reduction in metastasis, complete remission, partial remission, pathologic complete response, or stable disease.

75. The method of claim 73, wherein an improvement of clinical benefit rate (CBR=CR+PR+SD≧6 months) is obtained as compared to treatment with the anti-tumor agent but without the PARP inhibitor.

76. The method of claim 75, wherein the improvement of clinical benefit rate is at least about 60%.

77. The method of claim 73, wherein the uterine cancer is a metastatic uterine cancer.

78. The method of claim 73, wherein the uterine cancer is an endometrial cancer.

79. The method of claim 73, wherein the uterine cancer is recurrent, advanced, or persistent.

80. The method of claim 73, wherein the ovarian cancer is a metastatic ovarian cancer.

81. The method of claim 73, wherein the ovarian cancer is deficient in homologous recombination DNA repair.

82. The method of claim 73, wherein the uterine cancer is deficient in homologous recombination DNA repair.

83. The method of claim 73, wherein the uterine cancer is BRCA deficient.

84. The method of claim 73, wherein the ovarian cancer is BRCA deficient.

85. The method of claim 83 or 84, wherein the BRCA-deficiency is a BRCA1-deficiency, or BRCA2-deficiency, or both BRCA1 and BRCA2-deficiency.

86. The method of claim 73, wherein the PARP inhibitor is a benzamide or a metabolite thereof.

87. The method of claim 73, wherein the PARP inhibitor is 4-iodo-3-nitrobenzamide or a metabolite thereof.

88. The method of claim 73, wherein the PARP inhibitor is of Formula (IIa) or a metabolite thereof: 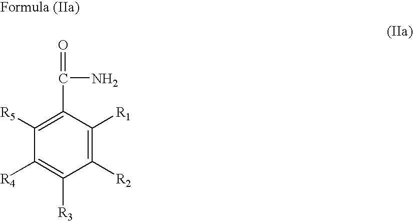
wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

89. The method of claim 73, wherein the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof.

90. The method of claim 73, wherein the anti-tumor agent is citabine, capecitabine, valopicitabine or gemcitabine.

91. The method of claim 73, wherein the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871.

92. The method of claim 73 further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

93. The method of claim 73, further comprising selecting a treatment cycle of at least 11 days and:
(a) on from 1 to 5 separate days of the cycle, administering to the patient about 100 to about 2000 mg/m² of paclitaxel;
(b) on from 1 to 5 separate days of the cycle, administering to the patient about 10-400 mg/m² of carboplatin; and
(c) on from 1 to 10 separate days of the cycle, administering to the patient about 1-100 mg/kg of 4-iodo-3-nitrobenzamide.

94. The method of claim 93, wherein paclitaxel is administered as an intravenous infusion.

95. The method of claim 93, wherein carboplatin is administered as an intravenous infusion.

96. The method of claim 93, wherein 4-iodo-3-nitrobenzamide is administered orally or as a parenteral injection or infusion, or inhalation.

97. A method of treating ovarian cancer or uterine cancer in a patient in need of such treatment, comprising:
(a) obtaining a sample from the patient;
(b) testing the sample to determine whether the patient is BRCA deficient;
(c) if the testing indicates that the patient is BRCA-deficient, treating the patient with at least one PARP inhibitor and at least one anti-tumor agent.

98. The method of claim 97, wherein at least one therapeutic effect is obtained, said at least one therapeutic effect being reduction in size of a uterine tumor or an ovarian tumor, reduction in metastasis, complete remission, partial remission, pathologic complete response, or stable disease.

99. The method of claim 97, wherein an improvement of clinical benefit rate (CBR=CR+PR+SD≧6 months) is obtained as compared to treatment with the anti-tumor agent but without the PARP inhibitor.

100. The method of claim 99, wherein the improvement of clinical benefit rate is at least about 60%.

101. The method of claim 97, wherein the uterine cancer is a metastatic uterine cancer.

102. The method of claim 97, wherein the uterine cancer is an endometrial cancer.

103. The method of claim 97, wherein the uterine cancer is recurrent, advanced, or persistent.

104. The method of claim 97, wherein the ovarian cancer is a metastatic ovarian cancer.

105. The method of claim 97, wherein the ovarian cancer is deficient in homologous recombination DNA repair.

106. The method of claim 97, wherein the uterine cancer is deficient in homologous recombination DNA repair.

107. The method of claim 97, wherein the uterine cancer is BRCA deficient.

108. The method of claim 97, wherein the ovarian cancer is BRCA deficient.

109. The method of claim 107 or 108, wherein the BRCA-deficiency is a BRCA1-deficiency, or BRCA2-deficiency, or both BRCA1 and BRCA2-deficiency.

110. The method of claim 97, wherein the PARP inhibitor is a benzamide or a metabolite thereof.

111. The method of claim 97, wherein the PARP inhibitor is 4-iodo-3-nitrobenzamide or a metabolite thereof.

112. The method of claim 97, wherein the PARP inhibitor is of Formula (IIa) or a metabolite thereof: 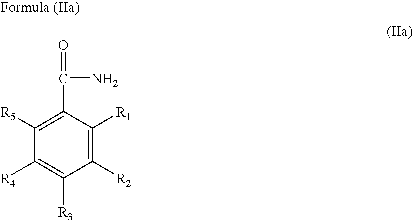
wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

113. The method of claim 97, wherein the sample is a tissue or bodily fluid sample.

114. The method of claim 97, wherein the sample is a tumor sample, a blood sample, a blood plasma sample, a peritoneal fluid sample, an exudate or an effusion.

115. The method of claim 97, wherein the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof.

116. The method of claim 97, wherein the anti-tumor agent is citabine, capecitabine, valopicitabine or gemcitabine.

117. The method of claim 97, wherein the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871.

118. The method of claim 97 further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

119. The method of claim 97, further comprising selecting a treatment cycle of at least 11 days and:
(a) on from 1 to 5 separate days of the cycle, administering to the patient about 100 to about 2000 mg/m² of paclitaxel;
(b) on from 1 to 5 separate days of the cycle, administering to the patient about 10-400 mg/m² of carboplatin; and
(c) on from 1 to 10 separate days of the cycle, administering to the patient about 1-100 mg/kg of 4-iodo-3-nitrobenzamide.

120. The method of claim 119, wherein paclitaxel is administered as an intravenous infusion.

121. The method of claim 119, wherein carboplatin is administered as an intravenous infusion.

122. The method of claim 119, wherein 4-iodo-3-nitrobenzamide is administered orally or as a parenteral injection or infusion, or inhalation.

123. A method of treating uterine cancer or ovarian cancer in a patient, comprising:
(a) obtaining a sample from the patient;
(b) testing the sample to determine a level of PARP expression in the sample;
(c) determining whether the PARP expression exceeds a predetermined level, and if so, administering to the patient at least one PARP inhibitor and at least one anti-tumor agent.

124. The method of claim 123, wherein at least one therapeutic effect is obtained, said at least one therapeutic effect being reduction in size of a uterine tumor or an ovarian tumor, reduction in metastasis, complete remission, partial remission, pathologic complete response, or stable disease.

125. The method of claim 123, wherein an improvement of clinical benefit rate (CBR=CR+PR+SD≧6 months) is obtained as compared to treatment with the anti-tumor agent but without the PARP inhibitor.

126. The method of claim 123, wherein the improvement of clinical benefit rate is at least about 60%.

127. The method of claim 123, wherein the PARP inhibitor is 4-iodo-3-nitrobenzamide or a metabolite thereof.

128. The method of claim 123, wherein the PARP inhibitor is of Formula (IIa) or a metabolite thereof: 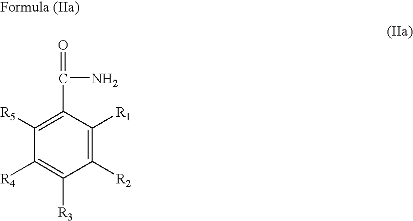
wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

129. The method of claim 123, wherein the sample is a tissue or bodily fluid sample.

130. The method of claim 123, wherein the sample is a tumor sample, a blood sample, a blood plasma sample, a peritoneal fluid sample, an exudate or an effusion.

131. The method of claim 123, wherein the uterine cancer is a metastatic uterine cancer.

132. The method of claim 123, wherein the uterine cancer is an endometrial cancer.

133. The method of claim 123, wherein the uterine cancer is recurrent, advanced, or persistent.

134. The method of claim 123, wherein the ovarian cancer is a metastatic ovarian cancer.

135. The method of claim 123, wherein the ovarian cancer is deficient in homologous recombination DNA repair.

136. The method of claim 123, wherein the uterine cancer is deficient in homologous recombination DNA repair.

137. The method of claim 123, wherein the uterine cancer is BRCA deficient.

138. The method of claim 123, wherein the ovarian cancer is BRCA deficient.

139. The method of claim 137 or 138, wherein the BRCA-deficiency is a BRCA1-deficiency, or BRCA2-deficiency, or both BRCA1 and BRCA2-deficiency.

140. The method of claim 123, wherein the anti-tumor agent is an antitumor alkylating agent, antitumor antimetabolite, antitumor antibiotics, plant-derived antitumor agent, antitumor platinum complex, antitumor campthotecin derivative, antitumor tyrosine kinase inhibitor, monoclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, anti-angiogenic agent, receptor tyrosine kinase inhibitor, topoisomerase inhibitor, taxane, agent targeting Her-2, hormone antagonist, agent targeting a growth factor receptor, or a pharmaceutically acceptable salt thereof.

141. The method of claim 123, wherein the anti-tumor agent is citabine, capecitabine, valopicitabine or gemcitabine.

142. The method of claim 123, wherein the anti-tumor agent is selected from the group consisting of Avastin, Sutent, Nexavar, Recentin, ABT-869, Axitinib, Irinotecan, topotecan, paclitaxel, docetaxel, lapatinib, Herceptin, tamoxifen, progesterone, a steroidal aromatase inhibitor, a non-steroidal aromatase inhibitor, Fulvestrant, an inhibitor of epidermal growth factor receptor (EGFR), Cetuximab, Panitumimab, an inhibitor of insulin-like growth factor 1 receptor (IGF1R), and CP-751871.

143. The method of claim 123 further comprises surgery, radiation therapy, chemotherapy, gene therapy, DNA therapy, adjuvant therapy, neoadjuvant therapy, viral therapy, RNA therapy, immunotherapy, nanotherapy or a combination thereof.

144. The method of claim 123, further comprising selecting a treatment cycle of at least 11 days and:
(a) on from 1 to 5 separate days of the cycle, administering to the patient about 100 to about 2000 mg/m² of paclitaxel;
(b) on from 1 to 5 separate days of the cycle, administering to the patient about 10-400 mg/m² of carboplatin; and
(c) on from 1 to 10 separate days of the cycle, administering to the patient about 1-100 mg/kg of 4-iodo-3-nitrobenzamide.

145. The method of claim 144, wherein paclitaxel is administered as an intravenous infusion.

146. The method of claim 144, wherein carboplatin is administered as an intravenous infusion.

147. The method of claim 144, wherein 4-iodo-3-nitrobenzamide is administered orally or as a parenteral injection or infusion, or inhalation.
